(19) United States
(12) Patent Application Publication
(10) Pub. No.: US 2023/0285338 A9
(43) Pub. Date: Sep. 14, 2023

(54) METHODS FOR TREATING MICROGLIAL DYSFUNCTION

(71) Applicant: Washington University, St. Louis, MO (US)

(72) Inventors: Marco Colonna, St. Louis, MO (US); Tyler Ulland, St. Louis, MO (US)

(21) Appl. No.: 17/840,368

(22) Filed: Jun. 14, 2022

Related U.S. Application Data
(63) Continuation-in-part of application No. 17/395,357, filed on Aug. 5, 2021.
Application No. 17/395,357 is a continuation of application No. 16/030,793, filed on Jul. 9, 2018, now abandoned.
(60) Provisional application No. 62/529,753, filed on Jul. 7, 2017.

(51) Int. Cl. A61K 31/195; C12Q 1/6827; A61P 25/28; C12Q 1/6883

(52) U.S. Cl. CPC A61K 31/195; C12Q 1/6827; A61P 25/28; C12Q 1/6883; C12Q 2600/156

(57) ABSTRACT

The present disclosure is directed to a method for treating a microglial dysfunction-associated neurodegenerative disease in a subject in need thereof. The method includes administering to the subject a therapeutically effective amount of a composition comprising a microglia receptor agonist that directly activates SYK.

### CROSS-REFERENCE TO RELATED APPLICATIONS

This application is a continuation-in-part of U.S. application Ser. No. 17/395,357, filed Aug. 5, 2021, which is a continuation of U.S. application Ser. No. 16/030,793, filed Jul. 9, 2018, now abandoned, and claims the benefit of U.S. Provisional Application No. 62/529,753, filed Jul. 7, 2017, the disclosures of which are hereby incorporated by reference in their entirety

### STATEMENT REGARDING FEDERALLY SPONSORED RESEARCH & DEVELOPMENT

This invention was made with government support under grant numbers AG051485, AG005681, and CA009547 awarded by National Institutes of Health. The government has certain rights in the invention.

### SEQUENCE LISTING

This application contains a sequence listing in computer readable format, the teachings and content of which are hereby incorporated by reference.

### FIELD OF THE DISCLOSURE

The field of the disclosure relates generally to compositions and methods of treating microglial dysfunction associated disease, disorder, or condition.

### BACKGROUND OF THE DISCLOSURE

Alzheimer's disease (AD) is the most common cause of late onset dementia. AD lesions in the CNS include plaques of amyloid β (Aβ) peptides and neurofibrillary tangles of hyperphosphorylated tau protein, both linked to synapse loss, neuronal death, and ultimately cognitive decline. Rare familial AD is due to mutations in amyloid precursor protein (APP) and presenilins (PS) that promote the generation of Aβ peptides prone to aggregation. However, the risk for common late-onset AD is associated with rare variants of immune receptors expressed on microglia. One of these receptors, TREM2, recognizes phospholipids, apoptotic cells, and lipoproteins. TREM2 transmits intracellular signals through two adapters, DAP12 and DAP10, which recruit protein tyrosine kinase Syk and phosphatidylinositol 3-kinase (PI3-K), respectively. Arginine-to-histidine variants at position 47 (R47H) or 62 (R62H) of TREM2 increase the risk for sporadic AD and impair binding to phospholipid ligands. These variants, as well as TREM2 deficiency and haploinsufficiency in mouse models of AD, moderate microglial proliferation, survival, and accumulation around Aβ plaques, thereby facilitate Aβ plaque buildup and injury of adjacent neurons. TREM2 has also been implicated in microglial phagocytosis of dead neurons, damaged myelin, and Aβ plaques. However, why defective TREM2 function or expression affects microglia responses to AD lesions is not known.

Moreover, microglia that adhere to Aβ plaques acquire a complex transcriptional profile, “disease-associated microglia” (DAM), which is partially dependent on the receptor complex TREM2-DAP12 that transmits intracellular signals through the protein tyrosine kinase SYK. The human TREM2R47H variant associated with high AD risk fails to activate microglia via SYK. SYK-deficient microglia cannot encase Aβ plaques, resulting in accelerated brain pathology and behavioral deficits. SYK deficiency impaired the PI3K-AKT-mTOR pathway, affecting energetic and anabolic support required for acquisition of the DAM profile.

Accordingly, there is a need for systemically-administered therapeutic treatments that rescue microglia responses in deficient and/or defective microglia and consequently prevent accumulation of Aβ plaques.

### BRIEF DESCRIPTION OF THE DISCLOSURE

Among the various aspects of the present disclosure is the provision of methods of treating microglial dysfunction-associated diseases, disorder, and conditions. The present disclosure provides for a method for treating microglial dysfunction in a subject having a microglial dysfunction-associated neurodegenerative disease comprising administering to a subject a therapeutically effective amount of composition including a microglial rescuing agent or a microglia receptor agonist. The present disclosure also provides for a method of reversing neuronal damage in a subject having a microglial dysfunction-associated neurodegenerative disease, and a method of treating at least one symptom of cognitive dysfunction in a subject having a microglial dysfunction-associated neurodegenerative disease.

In one aspect, the present disclosure is directed to a method for treating a microglial dysfunction-associated neurodegenerative disease in a subject in need thereof. The method comprises administering to the subject a therapeutically effective amount of a composition comprising a microglia receptor agonist that directly activates SYK.

In some embodiments, the microglial dysfunction-associated neurodegenerative disease is Alzheimer's disease, the subject has TREM2 deficient cells in the brain prior to administration of the composition, the microglial dysfunction-associated neurodegenerative disease is characterized by a mutation in Trem2 resulting in decreased microglial activity, and/or administering the therapeutically effective amount of the composition results in one or more of improved microglial metabolic activity, decreased microglial autophagy, reduced neurite dystrophy, decreased cell death, improved microglia viability or improved microglia numbers. In some embodiments, administering the therapeutically effective amount of the composition results in improved microglia clustering around amyloid beta plaques or reduced plaque-associated neurite dystrophy, the microglia receptor agonist that directly activates SYK comprises a CLEC7A agonist or pharmaceutically acceptable salt thereof, and/or the subject is human.

In another aspect, the present disclosure is directed to a method of reversing neuronal damage in a subject having a microglial dysfunction-associated neurodegenerative disease, wherein the microglial dysfunction-associated neurodegenerative disease is characterized by a mutation in Trem2 affecting microglial functions. The method comprises administering to the subject a therapeutically effective amount of a composition comprising a microglia receptor agonist that directly activates SYK.

In some embodiments, the subject has TREM2 deficient cells in the brain prior to administration of the composition, and/or administering the therapeutically effective amount of the composition results in one or more of improved microglial metabolic activity, decreased microglial autophagy, reduced neurite dystrophy, decreased cell death, improved microglia viability or improved microglia numbers. In some embodiments, the microglial dysfunction-associated neurodegenerative disease is Alzheimer's disease, administering the therapeutically effective amount of the composition results in improved microglia clustering around amyloid beta plaques or reduced plaque-associated neurite dystrophy, the microglia receptor agonist that directly activates SYK comprises a CLEC7A agonist or pharmaceutically acceptable salt thereof, and/or wherein the subject is human.

In yet another aspect, the present disclosure is directed to a method of treating at least one symptom of cognitive dysfunction in a subject having a microglial dysfunction-associated neurodegenerative disease, wherein the microglial dysfunction-associated neurodegenerative disease is characterized by a mutation in Trem2 affecting microglial functions. The method comprises administering to the subject a therapeutically effective amount of a composition comprising a microglia receptor agonist that directly activates SYK or activates at least one ITAM pathway.

In some embodiments, the at least one symptom is selected from a short term memory function and a spatial learning dysfunction, the subject has TREM2 deficient cells in the brain prior to administration of the composition, the microglia receptor agonist that directly activates SYK comprises a CLEC7A agonist or pharmaceutically acceptable salt thereof, and/or the subject is human.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The embodiments described herein may be better understood by referring to the following description in conjunction with the accompanying drawings.

FIG. 1A, FIG. 1B, FIG. 1C, FIG. 1D, FIG. 1E and FIG. 1F depict a defect in TREM2 enhances autophagy in vivo in the 5XFAD mouse model and in AD patients. FIG. 1A CD45⁺, CD11b⁺, F4/80⁺ cells were sorted from mouse brains of WT, Trem2^{-/-}, 5XFAD, and Trem2^{-/-} 5XFAD mice. TEM images of microglia sorted from 8-month-old WT, Trem2^{-/-}, 5XFAD, and Trem2^{-/-} 5XFAD mice. FIG. 1B average number of multivesicular and multilamellar structures/cell (30 cells analyzed/genotype). FIG. 1C confocal images of plaque bearing regions of the cortex (1.1 mm Bregma to 0.8 mm Bregma) of 8-month-old WT, Trem2^{-/-}, 5XFAD, and Trem2^{-/-} 5XFAD mice show Iba-1⁺ microglia (red), methoxy X04⁺ plaques (blue), and LC3 (green). Z-stacks composed of ˜30 images taken at 1.2 μm intervals were analyzed. Results are reported as an average of 2 regions of interest (ROI) analyzed. FIG. 1D quantification of the % of microglia that are positive for LC3 puncta. ˜150-400 microglia/HPF were analyzed depending on the genotype of the animal. FIG. 1E confocal images of sections from post-mortem brains of R47H⁺ AD patients and case-matched controls (CV, common variant of TREM2) show Iba-1⁺ microglia (red), methoxy X04⁺ plaques (blue), and LC3 (green). 3 ROIs/donor were analyzed and a total of between 400 and 700 microglia/individual were analyzed. FIG. 1F percentages of LC3+ microglia in post mortem specimens of AD patients with different genotypes. ***p<0.005, ****p<0.001 by One-way ANOVA with Holm-Sidak's multiple comparisons test. 15 cells from 2 separate mice were visualized for TEM (FIG. 1A, FIG. 1B). Confocal images are representative of 3 female mice per group (FIG. 1C) or 7 R47H, 4 R62H, and 8 case matched AD patients for post-mortem specimens (FIG. 1E). Immunoblots are representative of 3 independent experiments from microglia from 3 separate mice per group (FIG. 1G). Arrowheads indicate multilamellar and multivesicular structures (FIG. 1A) or LC3+ vesicles (FIG. 1C, FIG. 1E). See also FIG. 8 and Table 1.

FIG. 2A, FIG. 2B, FIG. 2C, FIG. 2D, FIG. 2E and FIG. 2F depict a defect in TREM2 impairs mTOR activation and elicits AMPK activation, autophagy and cell death in microglia from 5XFAD mice. FIG. 2A microglia were sorted as in FIG. 1A. Immunoblots for LC3I/II, p62, phosphoserine 473 AKT, phospho-AMPK, phospho-NDRG1, phospho-4EBP1, phospho-757 ULK1, and β-actin were performed on cell lysates. FIG. 2B quantification of the LC3II/I ratio in microglia from different genotypes. FIG. 2C single cell suspensions of brain tissue were incubated with MitoTraker Green and stained for CD45⁺, CD11b⁺, F4/80⁺. Representative histograms comparing unstained cells and microglia from 5XFAD and Trem2^{-/-} 5XFAD mice are shown. FIG. 2D quantification of the geometric mean fluorescence intensity (gMFI) of microglia from 3 mice of each genotype is shown. FIG. 2E confocal images of brain sections of 8 month-old WT, Trem2^{-/-} 5XFAD, and Trem2^{-/-} 5XFAD mice were taken as in FIG. 1C. Images depict Iba-1⁺ microglia (red), methoxy X04⁺ plaques (blue), and cleaved caspase-3 (green). FIG. 2F quantification of the % of LC3⁺ microglia that are positive for cleaved caspase-3. ****p<0.001 by One-way ANOVA with Holm-Sidak's multiple comparisons test (FIG. 2B and FIG. 2F). **p<0.01 by Student's T test (FIG. 2D). Immunoblots are representative of 3 independent experiments from microglia from 3 separate mice per group (FIG. 2A). Confocal images are representative of 3 female mice per group (FIG. 2E). See also FIG. 9.

FIG. 3A, FIG. 3B, FIG. 3C, FIG. 3D, FIG. 3E, FIG. 3F, FIG. 3G, FIG. 3H, FIG. 3I, FIG. 3J and FIG. 3K depict TREM2 deficiency affects mTOR signaling and induces autophagy in BMDM. FIG. 3A TEM images of WT and Trem2^{-/-} BMDM cultured overnight in either in 10% or 0.5% LCCM as source of CSF1. FIG. 3B number of multivesicular structures/cell observed in the TEM images 30 cells/genotype and condition were analyzed. FIG. 3C quantification of the LC3II/LC3I ratio in BMDMs from WT and Trem2^{-/-} mice cultured in 10% or 0.5% LCCM overnight or starved in HBSS for 4 hours prior to lysis. FIG. 3D immunoblots for LC3 and actin performed on lysates from WT and Trem2^{-/-} BMDMs cultured in 10% or 0.5% LCCM overnight. Cell were treated with bafilomycin were treated for 5 hours prior to harvest at a final concentration of 0.5 μg/ml. FIG. 3E quantification of LC3II/LC3I ratio in BMDMs from WT and Trem2^{-/-} mice treated as indicated. FIG. 3F-FIG. 3H immunoblots for phosphorylated Akt, NDRG1, S6K, 4EBP1, AMPK, Ulk1 and relative controls. Lysates were from WT and Trem2^{-/-} BMDM cultured overnight in 10% or 0.5% LCCM. FIG. 3I immunoblots for phosphorylated Akt, NDRG1, S6K, 4EBP1, mTOR, total S6K, Akt, and actin performed on lysates from WT and Trem2^{-/-} BMDMs cultured overnight in 10% or 0.5% LCCM followed by the addition of wortmannin for 3 hours prior to harvest. FIG. 3J and FIG. 3K immunoblots for LC3 and phosphoserine 473 AKT in WT and Trem2^{-/-} BMDM cultured in 10% LCCM with the indicated concentration of tunicamycin. Bar graph shows LC3II/LC3I ratios. Error bar represents mean±SEM. *p<0.05, **p<0.01, or ****p<0.001 by One-way ANOVA with Holm-Sidak's multiple comparisons test (FIG. 3B, FIG. 3C, FIG. 3E, FIG. 3K). Data are representative of at least 3 independent experiments. Arrowheads indicate multilamellar and multivesicular structures (FIG. 3A). See also FIG. 10.

FIG. 4A, FIG. 4B, FIG. 4C, FIG. 4D, FIG. 4E, FIG. 4F and FIG. 4G depict TREM2 deficiency reduces anabolic and energetic metabolism in BMDM. FIG. 4A top most changed metabolites between WT and Trem2^{-/-} BMDM cultured overnight in 10% LCCM. Defined as p<0.01 and identified in the mouse metabolic network analysis in B. FIG. 4B shiny-genes and metabolites (GAM) output for network analysis combining mass spectrometry and RNA-seq data highlights differences between WT and Trem2^{-/-} BMDM cultured in 10% LCCM. Enzyme-encoding mRNAs and metabolites downregulated or upregulated in Trem2^{-/-} cells vs WT cells are indicated with green or red nodes and connecters, respectively. FIG. 4C top most changed metabolites between WT and Trem2^{-/-} BMDM cultured in 0.5% LCCM. Defined as p<0.01 and identified in the mouse metabolic network analysis in FIG. 11C. FIG. 4D ATP content of WT and Trem2^{-/-} BMDM cultured in the indicated concentration of LCCM overnight. FIG. 4E extracellular acidification rate (ECAR) and baseline oxygen consumption rate (OCR) by WT and Trem2^{-/-} BMDM cultured overnight in the indicated concentration of LCCM. FIG. 4F and FIG. 4G mitochondrial mass of WT and Trem2^{-/-} BMDM assessed by Mito Tracker Green incorporation and by the ratio of mitochondrial-to nuclear DNA. Error bar represents mean±SEM. *p<0.05, ** p<0.01, or ****p<0.001 by One-way ANOVA with Holm-Sidak's multiple comparisons test (FIG. 4C) or Student's T test (FIG. 4F, FIG. 4G). Data are representative of at least 3 independent experiments. See also FIG. 11.

FIG. 5A, FIG. 5B, FIG. 5C, FIG. 5D, FIG. 5E, FIG. 5F and FIG. 5G depict enhanced energy storage or dectin-1 signaling can compensate for TREM2 deficiency. FIG. 5A ECAR of WT and Trem2^{-/-} BMDM incubated overnight in 0.5% LCCM±10 mM cyclocreatine. FIG. 5B viability of WT and Trem2^{-/-} BMDM incubated for 40 hours in 0.5% LCCM ±cyclocreatine. FIG. 5C Immunoblots of LC3, phosphorylated mTOR, phosphorylated Akt, and actin in WT and Trem2^{-/-} BMDM incubated overnight in 0.5% LCCM±5 mM cyclocreatine. FIG. 5D and FIG. 5F LC3, phosphoserine 473 AKT, p62, and actin immunoblots from WT and Trem2^{-/-} BMDM incubated overnight in the indicated concentration of LCCM±depleted zymosan. FIG. 5E Quantification of the LC3II/LC3I ratio derived from immunoblots of LC3 as shown in D. FIG. 5G ATP content of WT and Trem2^{-/-} BMDM cultured in the indicated concentration of LCCM±zymosan overnight. *p<0.05 or ****p<0.001 by One-way ANOVA with Holm-Sidak's multiple comparisons test (FIG. 5B, FIG. 5E, FIG. 5G). Data are representative of results from at least 3 independent experiments.

FIG. 6A, FIG. 6B, FIG. 6C, FIG. 6D, FIG. 6E and FIG. 6F depict enhanced energy storage can compensate for TREM2 deficiency in vivo. FIG. 6A TEM images of microglia sorted from 8-month-old 5XFAD, and Trem2^{-/-} 5XFAD mice ±cyclocreatine. FIG. 6B quantification of the number of multivesicular and multilamellar structures/cell from A. FIG. 6C confocal images of brain sections of 8-month-old 5XFAD, and Trem2^{-/-} 5XFAD mice±cyclocreatine show Iba-1⁺ microglia (red), methoxy X04⁺ plaques (blue), and LC3 (green). FIG. 6D clustering analysis quantifying the number of microglia per mm³ within 15 μm of the surface of plaques. FIG. 6E quantification of the number of LC3 puncta per HPF in the cortexes of the indicated mice. FIG. 6F quantification of the percentage of microglia that were cleaved caspase-3 positive from the indicated mice. *p<0.05, ***p<0.005 and ****p<0.001 by One-way ANOVA with Holm-Sidak's multiple comparisons test (FIG. 6B, FIG. 6D-FIG. 6F) results pooled from 2 independent experiments representing a total of 5-8 male and female mice per treatment group. Arrowheads indicate multilamellar and multivesicular structures (FIG. 6A) or LC3⁺ vesicles (FIG. 6C). See also FIG. 12.

FIG. 7A, FIG. 7B, FIG. 7C, FIG. 7D, FIG. 7E, FIG. 7F and FIG. 7G depict Energy supplementation can compensate for TREM2 deficiency and decrease neuronal damage in vivo. FIG. 7A representative images depicting plaques (X04 in blue), nuclei (To-Pro3 in white), microglia (Iba-1 in red), and Spp1 (in green) staining in cortexes of mice from the indicated genotypes. FIG. 7B quantification of the percentage of microglia that were Spp1⁺ in the indicated genotypes of mice. Confocal images were taken as in FIG. 1C. FIG. 7C immunoblots performed on lysates of microglia sorted from the indicated genotype and treatment group of mice. Immunoblots for phosphorylated Akt, NDRG1, total LC3, Akt, and actin. FIG. 7D quantification of the LC3II/LC3I ratio observed in immunoblots from 3 mice of each of the indicated genotypes and treatment groups. FIG. 7E average intensity of the plaques observed in the cortexes of mice from the indicated genotypes and treatment groups. FIG. 7F representative images depicting plaques (X04 in blue), nuclei (To-Pro3 in white), and N-terminus APP (green) from the indicated mice and treatment groups. Confocal images were taken as in FIG. 1C. FIG. 7G quantification of the number of dystrophic neurites/plaque in the indicated mice and treatment group. N.S. indicates not significant, *p<0.05, and ****p<0.001 by One-way ANOVA with Holm-Sidak's multiple comparisons test (FIG. 7A, FIG. 7C, FIG. 7D, FIG. 7F) results pooled from 2 independent experiments representing a total of 5-8 male and female mice per treatment group. See also FIG. 12.

FIG. 8 depicts a wider field of view of LC3 in microglia. Related to FIG. 1. Lower magnification confocal images of cortexes of WT, Trem2^{-/-}, 5XFAD and Trem2^{-/-} 5XFAD mice. Confocal images are representative of 3 female mice per group.

FIG. 9A, FIG. 9B, FIG. 9C, FIG. 9D, FIG. 9E, FIG. 9F, FIG. 9G, FIG. 9H, and FIG. 9I depict TREM2 deficiency in an AD model affects microglia expression of genes involved in metabolic pathways and microglia from Trem2^{-/-} 5XFAD mice undergo more cell death. Related to FIG. 2. FIG. 9A Sqstm1 expression taken from microarrays of sorted microglia from 8 month old WT, Trem2^{-/-}, 5XFAD and Trem2^{-/-} 5XFAD mice. FIG. 9B top 10 pathways in IPA analysis of differentially expressed genes from 5XFAD and Trem2^{-/-} 5XFAD microglia. Negative log₁₀ p-values are shown. FIG. 9C gene enrichment plots for genes included in the eIF2, glycolysis, and mTOR signaling modules of IPA. Plots were generated utilizing gene-set enrichment analysis (GSEA) software. FIG. 9D-FIG. 9F heat maps comparing WT, 5XFAD, Trem2^{-/-}, and Trem2^{-/-} 5XFAD microglia for expression of genes included in the eIF2, glycolysis and mTOR signaling pathways. FIG. 9G illustration of the glycolytic pathway: proteins indicated in red correspond to genes upregulated in 5XFAD but not Trem2^{-/-} 5XFAD microglia compared to WT microglia. FIG. 9H mosaic of images depicting representative images of microglia (Iba1 red), plaques (methoxy-X04 blue), cleaved caspase-3 (aqua), LC3 (green), and total merged images from a 5XFAD and a Trem2^{-/-} 5XFAD animal. FIG. 9I quantification of the percentage of microglia that are both LC3 and cleaved caspase-3 positive. Microarray data represents analyses of microglia sorted from 3 WT, 4 Trem2^{-/-} 5XFAD, and 5 Trem2^{-/-} 5XFAD mice (FIG. 9A-FIG. 9G). Confocal images are representative of 3 female mice per group (FIG. 9H). ***p<0.005 by One-way ANOVA with Holm-Sidak's multiple comparisons test (FIG. 9I).

FIG. 10A and FIG. 10B depict Trem2 is activated in vitro and contributes to PI3K-dependent mTOR activation. Related to FIG. 3. FIG. 10A immunoblots for phosphorylated Akt, NDRG1, S6K, 4EBP1, mTOR, total S6K, Akt, and actin performed on lysates from WT and Trem2^{-/-} BMDMs cultured overnight in 10% or 0.5% LCCM followed by the addition of Ly294002 for 3 hours prior to harvest. FIG. 10B reporter cell assay assessing TREM2 activation in reporter cell line incubated at optimal and low serum conditions with or with soluble anti-TREM2. N.S. indicates not significant and ****p<0.001 by One-way ANOVA with Holm-Sidak's multiple comparisons test (FIG. 10B). Results are representative of at least 3 independent experiments.

FIG. 11A, FIG. 11B, FIG. 11C, FIG. 11D, FIG. 11E, FIG. 11F, FIG. 11G, FIG. 11H, FIG. 11I, FIG. 11J, FIG. 11K, FIG. 11L and FIG. 11M show macrophages and microglia from Trem2^{-/-} mice are less energetically active. Related to FIG. 4. FIG. 11A, FIG. 11B heatmaps representing the total metabolic profiles of WT compared to Trem2^{-/-} BMDMs culture overnight in 10% LCCM (A) or 0.5% LCCM (FIG. 11B). FIG. 11C Shiny-GAM output for network analysis combining RNA-seq and mass spectrometry data highlights differences between WT and Trem2^{-/-} BMDM cultured overnight in 0.5% LCCM. Enzymes-encoding mRNAs and metabolites that are downregulated or upregulated in Trem2^{-/-} cells vs. WT cells are represented by green or red nodes and connecters, respectively. FIG. 11D quantification of phosphocreatine from WT and Trem2^{-/-} BMDMs cultured in the indicated concentration of LCCM. FIG. 11E, FIG. 11F assessment and quantification of the mitochondrial mass of resident peritoneal macrophages from WT and Trem2^{-/-} mice by MitoTraker Green incorporation. FIG. 11G, FIG. 11H assessment and quantification of the mitochondrial mass of thioglycolate elicited peritoneal macrophages from WT and Trem2^{-/-} mice by MitoTracker Green incorporation. FIG. 11I ATP content of WT and Trem2^{-/-} microglia cultured in 10% LCCM overnight. FIG. 11J mitochondrial content of WT and Trem2^{-/-} microglia assessed by the ratio of mitochondrial-to nuclear DNA. FIG. 11K extracellular acidification rate (ECAR) of WT and Trem2^{-/-} microglia cultured overnight in 10% LCCM. FIG. 11L primary WT and Trem2^{-/-} microglia were incubated overnight in the indicated concentration of LCCM. Immunoblots for LC3, p757 Ulk1, p317 Ulk1, p473 Akt, pNDRG1, and β actin were performed. FIG. 11M quantification of the LC3II/LC3I ratio from immunoblot performed on primary microglia shown in FIG. 11L. *p<0.05 or ****p<0.001 by One-way ANOVA with Holm-Sidak's multiple comparisons test (FIG. 11 FIG. 11, FIG. 11F, FIG. 11I, FIG. 11H, FIG. 11K). Results are representative of at least 3 independent experiments (FIG. 11E-FIG. 11K) or 2 independent experiments (FIG. 11L, FIG. 11M).

FIG. 12A, FIG. 12B, FIG. 12C, FIG. 12D, FIG. 12E, FIG. 12F, FIG. 12G, FIG. 12H, and FIG. 12I depict enhanced energy storage improves microglial response in Trem2^{-/-} 5XFAD mice in vivo. Related to FIG. 6 and FIG. 7. FIG. 12A quantification of the number of microglia per high-powered field in the cortexes of brains from mice of the indicated genotype and treatment group. FIG. 12B quantification of the percentage of microglia that contain LC3 puncta in the cortexes of brains from mice of the indicated genotype and treatment group. FIG. 12C quantification of the number of LC3 puncta per LC3⁺ microglia. FIG. 12D quantification of the relative enrichment of LC3⁺ microglia within 15 μm of a plaque surface in the cortexes of brains from mice of the indicated genotype and treatment group. FIG. 12E quantification of the relative enrichment of cleaved caspase-3⁺ microglia within 15 μm of a plaque surface in the cortexes of brains from mice of the indicated genotype and treatment group. FIG. 12F and FIG. 12G the percent of the overall area of the cortex (FIG. 12F) and hippocampus (FIG. 12G) 1.5× brighter than the mean in methoxy-X04 stained sections from mice of the indicated genotype and treatment group. FIG. 12H quantification of the percentage of microglia containing methoxy-X04 in plaque bearing regions of the cortexes from mice of the indicated genotype and treatment group. FIG. 12I assessment of the complexity of plaques in the cortexes from mice of the indicated genotype and treatment group. N.S. indicates not significant, **p<0.01 or ****p<0.001 by One-way ANOVA with Holm-Sidak's multiple comparisons test (FIG. 12A, FIG. 12B, FIG. 12D, FIG. 12E). Results pooled from 2 independent experiments representing a total of 5-8 male and female mice per treatment group.

FIG. 13(A-H) is an exemplary embodiment of conditional deletion of Syk in microglia abolishes the capacity of microglial responses to Aβ plaques in accordance with the present disclosure. FIG. 13A: Schematic showing the strategy for tamoxifen-inducible ablation of Syk and resulting effects on downstream signaling. FIG. 13B and FIG. 13C: FACS analysis detecting SYK expression in distinct cell types of Sykfl/fl and Syk^{DMG} mice in 9-month-old mice. FIG. 13D: Representative confocal images of the cortex regions (−1.91 mm Bregma to −2.15 mm Bregma) from 9-month-old Syk^{fl/fl}- and Syk^{DMG}-5xFAD mice showing Iba1+ microglia (green) and methoxy-X04 labeled Aβ plaques (blue). FIG. 13E: Quantification of the number of microglia per mm², as well as percentage of Iba1+ and methoxy-X04+ areas within cortex. FIG. 13F: Representative confocal images of the cortex and hippocampal dentate gyrus regions from 9-month-old Syk^{fl/fl}- and Syk^{DMG}-5xFAD mice showing Iba1+ microglia (green), TO-PRO3-stained nuclei (red), and methoxy-X04 labeled Aβ plaques (blue). Z stack images with maximum projection were analyzed. White boxes indicate locations of microglia around plaques. FIG. 13G and FIG. 13H: Quantification of the density of microglia within a 15-μm shell around plaque surfaces as well as microglia plaque coverage within cortex (FIG. 13G) or hippocampus (FIG. 13H) regions from 6- and 9-month-old mice. Original magnification 40× (FIG. 13D), 20× (FIG. 13F). Each symbol represents data of one mouse with two brain sections for technical repeats. **, P<0.01, ****, P<0.001 by two-tailed unpaired t test (Mann Whitney, FIG. 13E), two-way ANOVA with Sidak's multiple comparisons test (FIG. 13G and FIG. 13H). Data are presented as mean±SEM.

FIG. 14(A-J) is an exemplary embodiment of SYK deficiency in microglia exacerbates AJ3 pathology and leads to behavior and memory defects in accordance with the present disclosure. FIG. 14A: Representative images showing AJ342 in 6- and 9-month-old Syk^{fl/fl}- and Syk^{AMG}-5xFAD mice. FIG. 14B: Quantification of the percentage of cortical area stained for AJ342. FIG. 14C: Representative confocal images of the cortex from 9-month-old of Syk^{fl/fl}- and Syk^{AMG}-5xFAD mice, showing methoxy-X04-labeled Aβ plaques (blue) and surrounding Lamp1+ dystrophic neurites (green). FIG. 14D: Quantification of volumes of dystrophic neurites per plaque from 6- or 9-month-old mice. FIG. 14E, FIG. 14F, and FIG. 14G: Analysis of water maze tests measuring learning ability by latency time to submerged platform (FIG. 14F) as well as spatial bias by time in target quadrant (FIG. 14G) in 6- and 9-month-old Sykfl/fl and Syk^{AMG} mice without or with 5xFAD background. FIG. 14H, FIG. 14I, and FIG. 14J: Elevated plus maze test for the indicated genotypes without or with 5xFAD background at 6 and 9 months of age, presented as percentage of entries into open arms (FIG. 14I) and percentage of travelled distance in open arms (FIG. 14J). Original magnification 4× (FIG. 14A), 20× (FIG. 14C). Each symbol represents data of one mouse with three (FIG. 14A) or two brain sections (FIG. 14C) for technical repeats. Data in FIG. 14F represent analyses of 6-month-old Sykfl/fl (n=6), Syk^{AMG} (n=7), Syk^{fl/fl}-5xFAD (n=12) and Syk^{AMG}-5xFAD (n=11), as well as 9-month-old Sykfl/fl (n=7), Syk^{AMG} (n=6), Syk^{fl/fl}-5xFAD (n=6) and Syk^{AMG}-5xFAD (n=9). *, P<0.05, **, P<0.01, ***, P<0.001 by two-way ANOVA with Sidak's multiple comparisons test (FIG. 14B, FIG. 14D, FIG. 14F, FIG. 14I and FIG. 14J), one-way ANOVA with Tukey's multiple comparisons test (FIG. 14G). Data are presented as mean±SEM.

FIG. 15(A-H) is an exemplary embodiment of how SYK defect impairs the PI3K-AKT-mTOR axis, and augments autophagy and lipids accumulation in microglia of 5xFAD mice in accordance with the present disclosure. FIG. 15A: Microglia were sorted from 9-month-old of Syk^{fl/fl}- and Syk^{AMG}-5xFAD mice. Immunoblots for Syk, LC3I/II, phosphorylated Akt (serine 473) and NDRG1. Actin were assessed on cell lysates for loading control. FIG. 15B: Immunoblots for Syk, phosphorylated Akt (serine 473), S6K and actin as control. Lysates were obtained from SYK-sufficient and SYK-deficient PM stimulated with the indicated concentration of LCCM for 10 min. FIG. 15C: Confocal images of brain sections of cortex and hippocampus from 9-month-old Syk^{fl/fl}- and Syk^{AMG}-5xFAD mice showing methoxy-X04 labeled Aβ plaques (blue), Iba1⁺ microglia (red) and LC3 (green). White arrows indicate microglial LC3⁺ vesicles. FIG. 15D: Quantification of the % of microglia stained for LC3. FIG. 15E: Representative TEM images of microglia (CD45 lo CD11b+) sorted from 9-month-old of Sykfl/fl and Syk^{AMG} mice without or with 5xFAD background. N indicates cellular nucleus and yellow arrows point multivesicular structures. FIG. 15F: Quantification of the number of multivesicular structures per microglia observed in TEM images (30 cells analyzed/genotype). FIG. 15G: Representative TEM images of lipid accumulation in microglia sorted from 9-month-old of Syk^{AMG} mice without or with 5xFAD background. N indicates cellular nucleus. FIG. 15H: Average number of large lipid structures per microglia of each genotype. 30 cells from each mouse genotype were analyzed. Large lipid structures are visible in Syk^{AMG} microglia as compared to Sykfl/fl microglia, and even more evident in Syk^{AMG}-5xFAD microglia. Original magnification 40× (FIG. 15B), 6000× (FIG. 15E and FIG. 15G), 20000× (FIG. 15G, zoom in). Each symbol represents data of one mouse with two brain sections for technical repeats (FIG. 15D) or one microglia (FIG. 15F and FIG. 15H). *, P<0.05, **, P<0.01, ***, P<0.001 by two-way ANOVA with Sidak's multiple comparisons test. Data are presented as mean±SEM.

FIG. 16(A-F) is an exemplary embodiment of SYK is required for maintenance of microglial clustering around the Aβ plaques in 5xFAD mice in accordance with the present disclosure. FIG. 16A: Schematic diagram showing the experimental set up for tamoxifen treatment and timepoints of analysis. FIG. 16B: Representative confocal images of brain sections within the cortex and hippocampus regions of 5- and 9-month-old Syk^{fl/fl}- and Syk^{DMG}-5xFAD mice were taken as in FIG. 13F. Images depict Iba1+ microglia (green), TO-PRO3-stained nuclei (red), and methoxy-X04 labeled Aβ plaques (blue). White circles indicate the contours of the plaques. FIG. 16C and FIG. 16D: Clustering analysis showing quantification of the density of microglia per mm3 within 15-μm shell around plaque surfaces (FIG. 16C) as well as microglia plaque coverage (FIG. 16D) in the cortex or hippocampus from 5- and 9-month-old mice. FIG. 16E: Representative confocal images of cortex and hippocampus sections show methoxy-X04+Aβ plaques (blue) and Pu.1⁺Iba1⁺ microglia (green and red). FIG. 16F: Quantification of the number of microglia per mm² within cortex or hippocampus regions from 5- and 9-month-old mice. Original magnification 40× (FIG. 16B and FIG. 16E). Each symbol represents data of one mouse with two brain sections for technical repeats (FIG. 16C and FIG. 16D). Data in FIG. 16F represent 5-month-old Syk^{fl/fl}-5xFAD (n=6) and Syk^{DMG}-5xFAD (n=4), as well as 9-month-old Syk^{fl/fl}-5xFAD (n=6) and Syk^{DMG}-5xFAD (n=8) mice. *, P<0.05, **, P<0.01, ***, P<0.001, ****, P<0.001 by two-way ANOVA with Sidak's multiple comparisons test (FIG. 16C and FIG. 16D), two-tailed unpaired t test (Mann Whitney, FIG. 16F). Data are presented as mean±SEM.

FIG. 17(A-L) is an exemplary embodiment of SYK deficiency in microglia hampers microglial activation in 5xFAD mice in accordance with the present disclosure. FIG. 17A: Schematic showing the processing of CD45+ cells from cortical tissues of Syk^{fl/fl}, Syk^{fl/fl}-5xFAD, Syk^{AMG}, and Syk^{AMG}-5xFAD mice for single-cell RNA-seq. FIG. 17B: UMAP plots of microglial clusters in each genotype. Cluster identities were based on expression of key markers shown in FIG. 22(A-D). FIG. 17C: Proportional contribution of each genotype to indicated microglial clusters. FIG. 17D and FIG. 17E: Representative confocal images of the cortex from 6-month-old Syk^{fl/fl}- and Syk^{AMG}-5xFAD mice, showing methoxy-X04-labeled Aβ plaques (blue), Iba-1 microglia (green) and CD11 c (red) in (FIG. 17D) or CD74 (red) in (FIG. 17E). FIG. 17F: Quantification of CD11c+ and CD74+ microglia in the cortex of Syk^{fl/fl}-5xFAD and Syk^{AMG}-5xFAD mice. FIG. 17G: Representative confocal images of the cortex from 6-month-old Syk^{fl/fl}- and Syk^{AMG}-5xFAD mice, showing methoxy-X04-labeled Aβ plaques (blue), Iba-1 microglia (red) and Ki67 (green). FIG. 17H: Quantification of Ki67+ microglia from indicated mice. FIG. 17I: Fractional distribution of Cluster 3 among Sye Syk^{fl/fl}-5xFAD, Syk^{AMG}, and Syk^{AMG}-5xFAD genotypes. FIG. 17J: Average scaled expression levels of selected enriched gene markers in the indicated clusters. FIG. 17K: UMAP plots of indicated clusters showing expression of indicated markers. FIG. 17L: Violin plots showing the expression scores of the DAM signature in the indicated clusters. Original magnification 40× (FIG. 17D and FIG. 17G), 20× (FIG. 17E). Each symbol represents data of one mouse with two brain sections for technical repeats. *, P<0.05, ***, P<0.001 by two-tailed unpaired t test (Mann Whitney). Data are presented as mean±SEM.

FIG. 18(A-L) is an exemplary embodiment of a comparison between scRNA-seq data of SYK- and TREM2-deficient microglia reveals distinct defects in DAM trajectories in accordance with the present disclosure. FIG. 18A: UMAP plots of microglial clusters in Syk^{fl/fl}-5xFAD, Syk^{DMG}-5xFAD, Trem2^{+/+}-5xFAD, and Trem2-5xFAD genotypes. FIG. 18B: Dot plots showing expression of marker genes by microglial cluster. FIG. 18C: Relative changes in frequency across each cluster comparing Syk^{DMG} and Trem2^{-/-} microglia with their respective controls. FIG. 18D: UMAP plot showing the projection of the three identified trajectories on microglial clusters. FIG. 18E: Histograms showing the distribution of cells from each genotype along DAM, IFN-R, and cycling trajectories. FIG. 18F: Proportion of cluster TM1 in Syk^{fl/fl}-5xFAD, Syk^{DMG}-5xFAD, Trem2^{+/+}-5xFAD, and Trem2^{-/-}-5xFAD genotypes. FIG. 18G: Expression of Apoe and Fabp5 expression along the DAM trajectory for Syk^{DMG}-5xFAD, Trem2^{-/-}-5xFAD, and 5xFAD microglia. FIG. 18H: Violin plots comparing the expression of Apoe between genotypes for selected microglial clusters. FIG. 18I, FIG. 18J, FIG. 18K, and FIG. 18L: Representative confocal images of the cortex from 6-month-old Trem2^{+/+}-, Trem2^{-/-}-5xFAD (FIG. 18I), Syk^{fl/fl}-, Syk^{DMG}-5xFAD (FIG. 18K), showing methoxy-X04-labeled Aβ plaques (blue), Iba-1 microglia (green) and ApoE (red) and the quantification of the percentage of ApoE-positive plaques from indicated mice (FIG. 18J and FIG. 18L). Original magnification 40×. Each symbol represents data from one mouse with two brain sections for technical repeats. *, P<0.05, ***, P<0.001 by two-tailed unpaired t test (Mann Whitney). Data are presented as mean±SEM.

FIG. 19(A-G) is an exemplary embodiment of acute treatment with anti-CLEC7A induces microglial activation in TREM2R47H-5xFAD mice in accordance with the present disclosure. FIG. 19A: Schematic diagram of anti-CLEC7A treatment in TREM2^{R47H}-5xFAD mice. FIG. 19B: Representative confocal images of the cortex regions from indicated mice showing Iba1+ microglia (green) and methoxy-X04 labeled Aβ plaques (blue) and TO-PRO3-stained nuclei (red). Images were captured as in FIG. 13F. FIG. 19C: Quantification of the density of microglia within a 15-μm shell around plaque surfaces and % of % Ibaa1⁺ areas within cortex regions from indicated mice. FIG. 19D: Representative confocal images of the cortex from indicated mice showing methoxy-X04-labeled Aβ plaques (blue), Iba-1 microglia (green) and CD11c (red). FIG. 19E: Quantification of the microglia that were CD11c-positive from indicated mice. FIG. 19F: mRNA expression of microglia activation marker Itgax in whole cortical tissue shows increase in anti-CLEC7A treated mice compared to the CTRL groups. FIG. 19G: tSNE plots of human brain showing the expression of SYK, CLEC7A and TREM2 in human microglia. Original magnification 40x (FIG. 19B and FIG. 19D). Each symbol represents data of one mouse with two brain sections for technical repeats. *, P<0.05, ***, P<0.001 by two-tailed unpaired t test (Mann Whitney). Data are presented as mean±SEM.

FIG. 20(A-D) is an exemplary embodiment of conditional deletion of Syk in microglia impairs Ab plaques-induced microgliosis in 5xFAD mice, related to FIG. 13(A-H) in accordance with the present disclosure. FIG. 20A: Schematic diagram showing the tamoxifen treatment timepoints and experimental design. FIG. 20B: Analysis of Iba1 intensity profile in the cortex regions of 9-month-old Sylc^{DMG}-5xFAD mice reveal a dramatic decrease in microgliosis compared to Syk^{fl/fl}-5xFAD controls. FIG. 20C: Representative confocal images with % al⁺ microglia (green) and methoxy-X04 labeled Aβ plaques (blue) and Pu.1 (red) showing the decreased number of plaques-associated microglia. FIG. 20D: Quantification of % of Iba1+ area, as well as the number of microglial cells per mm² in 9-month-old Sykfl/fl and Syk^{DMG} mice reveals no differences between these two genotypes. Original magnification 40×. Each symbol represents data of one mouse with two brain sections for technical repeats. Data are presented as mean±SEM.

FIG. 21(A-D) is an exemplary embodiment of SYK deficiency in microglia exacerbates AD pathology, related to FIG. 14(A-J) in accordance with the present disclosure. FIG. 21A: Representative confocal images of the cortex from 9-month-old of Sykfl/fl-5xFAD mice showing distinct forms of Ab plaques, including 6E10+(red) Methoxy-X04—/minor (green) filamentous plaques, 6E10+ Methoxy-X04+ dynamic plaques, and 6E10—/minor Methoxy-X04+ inert plaques. FIG. 21B: Quantification of the percentages of filamentous, inert, and dynamic plaques in each genotype of 6- or 9-month-old mice. A total of 1,890 plaques from 6-month-old or 1706 plaques from 9-month-old were analyzed, respectively. FIG. 21C: Analysis of water maze tests measuring latency time to visible platform in 6- and 9-month-old Sykfl/fl and SykDMG mice without or with 5xFAD background. FIG. 21D: Elevated plus maze test for the indicated genotypes without or with 5xFAD background at both 6 and 9 months of age, presented as total traveled distance during the tested time. Original magnification 40×. Each symbol represents data of one mouse with two brain sections (FIG. 21B) for technical repeats. Data in FIG. 21C represent analyses of 6-month-old Sykfl/fl (n=6), SykDMG (n=7), Sykfl/fl-5xFAD (n=12) and SykDMG-5xFAD (n=11), as well as 9-month-old Sykfl/fl (n=7), SykDMG (n=6), Sykfl/fl-5xFAD (n=6) and SykDMG-5xFAD (n=9) mice. *, P<0.05, **, P<0.01 by two-way ANOVA with Sidak's multiple comparisons test. Data are presented as mean±SEM.

FIG. 22(A-D) is an exemplary embodiment of identification of microglia clusters from scRNA-seq of CD45+ cells, related to FIG. 17(A-L) in accordance with the present disclosure. FIG. 22A: UMAP plot showing all CD45+ cells. FIG. 22B: Re-clustered microglial cells. FIG. 22C: Gene expression heatmap for microglial subsets. Genes shown are the top gene markers for each cluster. The number of cells per cluster is denoted above the cluster label. Clusters were annotated using previously identified gene markers: HM, homeostatic microglia; TM, transitioning microglia; IFN-R, interferon-response microglia; DAM, disease-associated microglia, MHC-II, MHC class II microglia. FIG. 22D: UMAP plots of selected cluster markers among microglial clusters.

### DETAILED DESCRIPTION OF THE DISCLOSURE

### Methods for Treating Microglial Dysfunction

Applicants have discovered that the use of a microglial rescuing agent which supplements microglial energetic metabolism can be an effective treatment for subjects with microglial dysfunction-associated diseases, disorder, and conditions.

Disclosed herein are components used to prepare disclosed compositions as well as the compositions themselves, and methods of use thereof. It is understood that when combinations, subsets, interactions, groups, etc. of these materials are disclosed, that while specific reference to each various individual and collective combinations and permutation may not be explicitly disclosed, each is specifically contemplated and described herein. For example, if a particular compound is disclosed and discussed and a number of modifications that can be made to a number of molecules of the compound are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated, meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Additional aspects of the invention are described below.

### (I) Compositions

One aspect of the present disclosure encompasses a microglial rescuing agent capable of mitigating one or more of the pathologies associated with microglial dysfunction. In some embodiments microglial dysfunction results from perturbations of cellular biosynthetic metabolism. In one aspect, microglial dysfunction results from impaired mTOR activation. In some embodiments, the present disclosure encompasses providing a therapeutically effective amount of one or more microglial rescuing agents, which results in improved metabolic activity, decreased autophagy, decreased cell death, improved microglia viability, improved microglia numbers or a combination thereof.

A composition of the invention may optionally comprise one or more additional drugs or therapeutically active agents in addition to the microglial rescuing agent. A composition of the invention may further comprise a pharmaceutically acceptable excipient, carrier, or diluent. Further, a composition of the invention may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents, or antioxidants.

The compositions as disclosed herein comprise microglial rescuing agents such as creatine compounds, a creatine analogs, and pharmaceutically acceptable salts thereof. Additionally, the compositions as disclosed herein comprise activators of the dectin-1 pathway, such as dectin-1 ligands. The present disclosure also provides pharmaceutical compositions. The pharmaceutical composition comprises a microglial rescuing agent, as an active ingredient, and at least one pharmaceutically acceptable excipient.

Other aspects of the invention are described in further detail below.

(a) Creatine Compounds

Creatine compounds useful in the present invention include compounds which modulate microglial metabolism. As described herein, creatine and derivatives/analogs thereof have been identified to rescue microglial function and treat microglial-dysfunction associated disease. Creatine and analogs thereof have been shown to supplement microglial energetic metabolism in subjects with microglial dysfunction, particularly those with a TREM2 or ApoE variant. Compounds which are effective for this purpose include creatine, creatine phosphate and analogs thereof, compounds which mimic their activity, and salts of these compounds as defined herein. Exemplary creatine compounds are described below.

Creatine (also known as N-(aminoiminomethyl)-N-methylglycine; methylglycosamine or N-methyl-guanido acetic acid) is a known substance. (See, The Merck Index, 60 Eleventh Edition, No. 2570 (1989).

Creatine is phosphorylated chemically or enzymatically by creatine kinase to generate creatine phosphate, which also is known (see, The Merck Index, No. 7315). Both creatine and creatine phosphate (phosphocreatine) can be extracted from animal tissue or synthesized chemically. Both are commercially available.

Cyclocreatine is an essentially planar cyclic analog of creatine. Although cyclocreatine is structurally similar to creatine, the two compounds are distinguishable both kinetically and thermodynamically. Cyclocreatine is phosphorylated efficiently by creatine kinase in the forward reaction both in vitro and in vivo. Rowley, G. L., J. Am. Chem. Soc. 93: 5542-5551 (1971); McLaughlin, A. C. et. al., J. Biol. Chem. 247, 4382-4388 (1972).

The phosphorylated compound phosphocyclocreatine is structurally similar to phosphocreatine; however, the phosphorous-nitrogen (P N) bond of cyclocreatine phosphate is more stable than that of phosphocreatine. LoPresti, P. and M. Cohn, Biochem. Biophys. Acta 998: 317-320 (1989); Annesley, T. M. and J. B. Walker, J. Biol. Chem. 253; 8120-8125, (1978); Annesley, T. M. and J. B. Walker, Biochem. Biophys. Res. Commun. 74: 185-190 (1977).

A creatine analog can be any creatine analog that targets the creatine kinase system or a creatine based composition. For example, a creatine analog can be cyclocreatine, phosphocreatine (aka creatine phosphate), nicotinamide mononucleotide (NMN), creatine ethyl ester, creatine nitrate, creatine gluconate, creation methyl ester, creatine riboside, creatine sulphate, serotonin creatine sulphate, creatine ethylester (HCl), creatine hydrochloride, creatine pyruvate, creatine citrate, creatine hemisulfate salt, creatine-(methyl-d3) monohydrate, creatine zinc chloride, creatine taurinate, 5,7-dihydroxytryptamine, L-arginine alpha-ketoglutarate, creatine pyroglutamate, creatine calcium, creatine magnesium, creation dextrose, creatine ethyl ester malate, or derivatives thereof. Exemplary compounds are shown below:

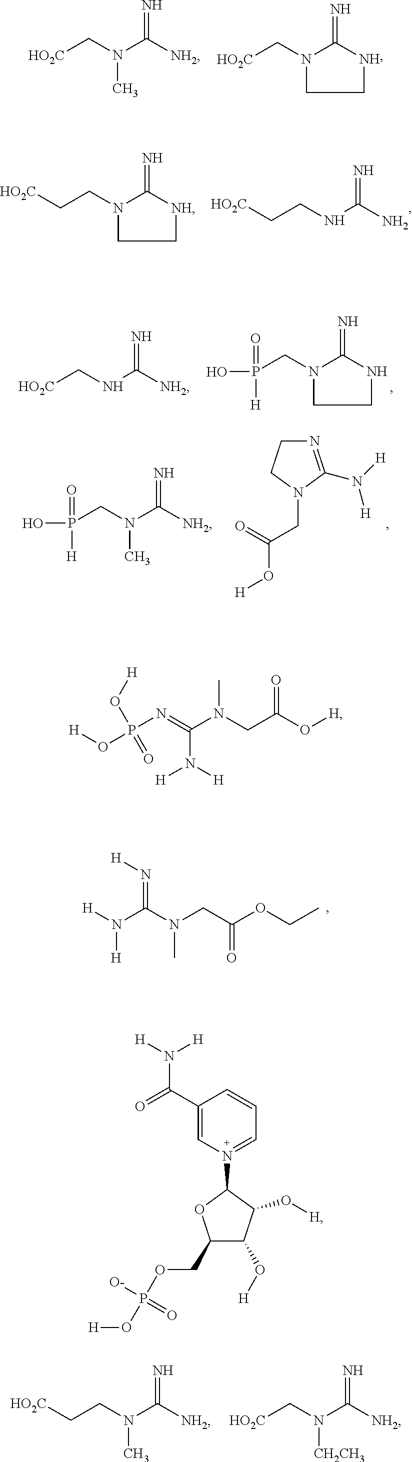

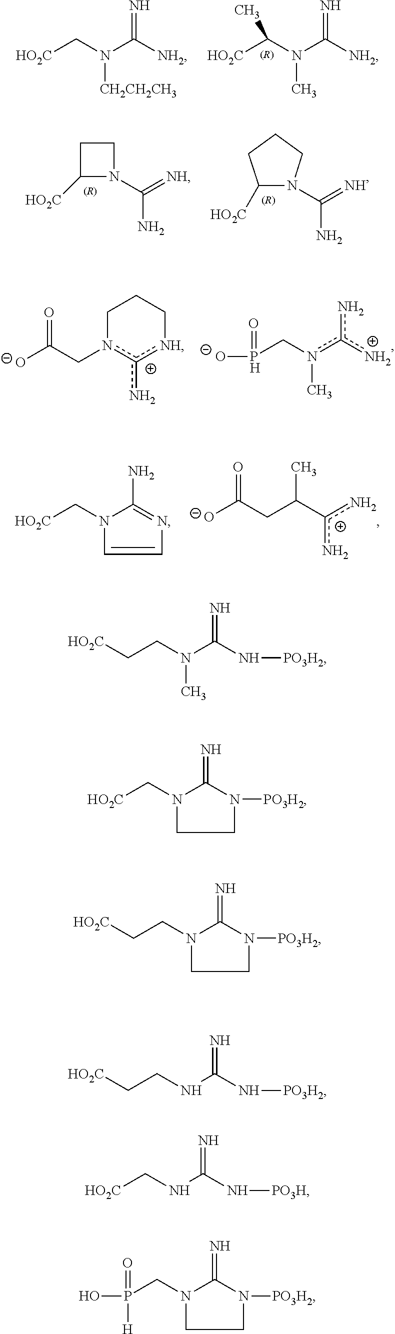

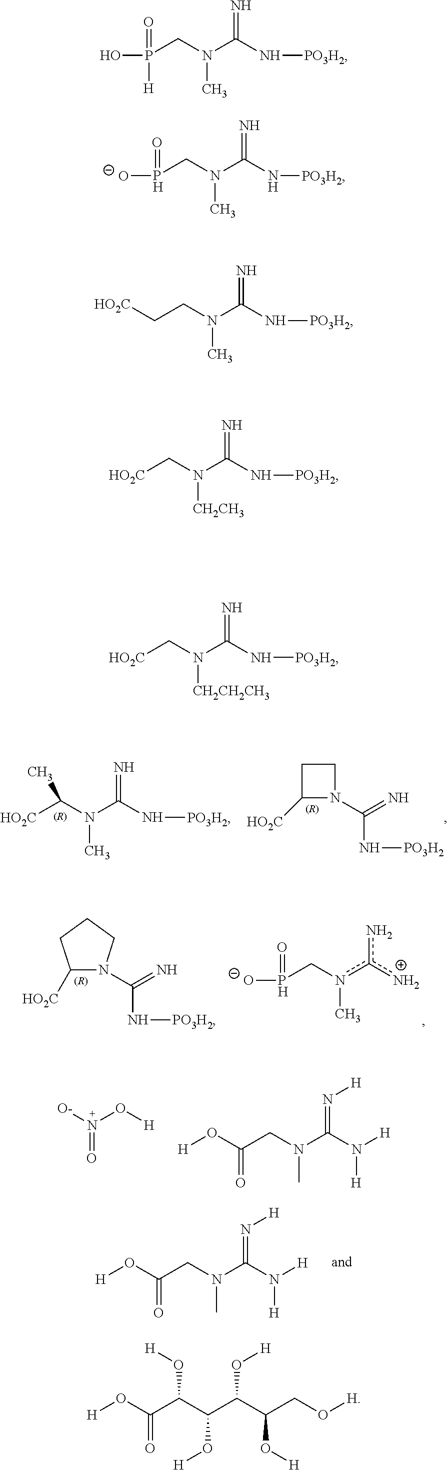

### (b) Dectin-1 Agonist

Dectin-1 (aka C-type lectin domain family 7 member A; UniProt accession number Q9BXN2) is a protein that in humans is encoded by the CLEC7A gene. Dectin-1 is a member of the C-type lectin/C-type lectin-like domain (CTL/CTLD) superfamily. The encoded glycoprotein is a small type II membrane receptor with an extracellular C-type lectin-like domain fold and a cytoplasmic domain with a partial immunoreceptor tyrosine-based activation motif. It functions as a pattern-recognition receptor for a variety of β-1,3-linked and β-1,6-linked glucans from fungi and plants, and in this way plays a role in innate immune response. Expression is found on myeloid dendritic cells, monocytes, macrophages and B cells. Alternate transcriptional splice variants, encoding different isoforms, have been characterized. This gene is closely linked to other CTL/CTLD superfamily members on chromosome 12p13 in the natural killer gene complex region. Dectin-1 is a transmembrane protein containing an immunoreceptor tyrosine-based activation (ITAM)-like motif in its intracellular tail (which is involved in cellular activation) and one C-type lectin-like domain (carbohydrate-recognition domain, CRD) in the extracellular region (which recognizes β-glucans and endogenous ligands on T cells). The CRD is separated from the membrane by a stalk region. CLEC7A contains putative N-linked sites of glycosylation in the stalk region.

The C-type lectin receptors are class of signaling pattern recognition receptors which are involved in antifungal immunity, but also play important roles in immune responses to other pathogens such as bacteria, viruses and nematodes. As a member of this receptor family, Dectin-1 recognizes β-glucans and carbohydrates found in fungal cell walls, some bacteria and plants, but may also recognize other unidentified molecules (endogenous ligand on T-cells and ligand on mycobacteria). Ligand binding induces intracellular signaling via the ITAM-like motif. CLEC7A can induce both Syk dependent or Syk independent pathways. Dimerization of dectin-1 upon ligand binding leads to tyrosine phosphorylation by Src family kinases and recruitment of Syk. Syk activates transcription factor NFκB. This transcription factor is responsible for the production of numerous inflammatory cytokines and chemokines such as TNF, IL-23, IL-6, IL-2. Other responses include: respiratory burst, production of arachidonic acid metabolites, dendritic cell maturation, and phagocytosis of the ligand.

The term “Dectin-1 agonist”, also referred to herein as “Dectin-1 ligand”, refers to a molecule that specifically binds to Dectin-1 resulting in activation of the Dectin-1 pathway. Activators of Dectin-1 signaling may be used alone, with other agents with similar or different effects or with other modalities, including surgery and the like.

In one aspect, this disclosure provides a microglial rescuing agent capable of activating the Dectin-1 signaling pathway. A Dectin-1 agonist is also referred to herein as a ligand. In one embodiment, the Dectin-1 agonistic is an antibody or a fragment thereof. In another embodiment, the Dectin-1 agonistic is a small molecule. Non-limiting examples of Dectin-1 ligands include beta-glucan peptide (BGP), curdlan AL, heat-killed C. albicans, heat-killed S. cerevisiae, laminarin, lichenan, pustulan, schizophyllan, scleroglucan, WGP Dispersible, Zymosan, Zymosan Depleted. These agonists are commercially available (Invivogen). Another example of Dectin-1 activator is vimentin. Another example of Dectin-1 activator is an agonistic anti-Dectin-1 antibody, such as, for example, an antibody described in U.S. Pat. No. 9,045,542, the description of which antibody is incorporated herein by reference. In one embodiment, a microglial rescuing agent is one or more of Dectin-1 agonists or activators.

In one aspect, this disclosure provides methods for identifying activators of Dectin-1 pathway in microglial cells. In one embodiment, the activators of Dectin-1 pathway are Dectin-1 agonists. The activity of a test agent may be evaluated based on the effect on any step of the Dectin-1 pathway (as described in this disclosure). It can be compared to the effect in the absence of the test compound or may be compared to the effect of Dectin-1 or a known agonist thereof.

Assays to evaluate agents for binding to Dectin-1 may be carried out by in vitro using purified or recombinant Dectin-1. Assays can also be carried out in vitro using cells which express Dectin-1--such as liver leukocytes or hepatic stellate cells. Further, screening test may be carried out in vivo using animal models. The cells in culture may be primary cells or may be secondary cells or cell lines. Examples of suitable cells include liver leukocytes (such as dendritic cells, macrophages, CD14⁺ monocytic cells and the like), and hepatic stellate cells. The cells may be enriched from sources such as whole blood. For example, whole blood may be obtained from an individual and desired types of leukocytes may be isolated using well known techniques or using commercially available kits (such as kits from Miltenyi Biotec). In one embodiment, the cells may be modified cells. For example, the cells may be engineered to express or overexpress Dectin-1. The cells in culture can be maintained by using routine cell culture reagents and procedures. In one embodiment, the assays may be carried out in animals including mice after administration of Thioacetamide (TAA) or Carbon tetrachloride.

The compounds for testing may be part of a library or may be newly synthesized. Further, the compounds may be purified, partially purified or may be present as cell extracts, crude mixtures and the like—i.e., unpurified. While it is ideal to test each compound separately, a combination of compounds may also be tested.

Dosages of a microglial rescuing agent can vary between wide limits, depending upon the disease or disorder to be treated, the age and condition of the subject to be treated. In an embodiment where a composition comprising a microglial rescuing agent is contacted with a sample, the concentration of a microglial rescuing agent may be from about 0.1 μM to about 40 μM. Alternatively, the concentration of a microglial rescuing agent may be from about 5 μM to about 25 μM. For example, the concentration of a microglial rescuing agent may be about 0.1, about 0.25, about 0.5, about 0.6, about 0.7, about 0.8, about 0.9, about 1, about 2.5, about 5, about 6, about 7, about 8, about 9, about 10, about 11, about 12, about 13, about 14, about 15, about 16, about 17, about 18, about 19, about 20, about 21, about 22, about 23, about 24, about 25, about 30, about 35, or about 40 μM. Additionally, the concentration of a microglial rescuing agent may be greater than 40 μM. For example, the concentration of a microglial rescuing agent may be about 40, about 45, about 50, about 55, about 60, about 65, about 70, about 75, about 80, about 85, about 90, about 95, or about 100 μM.

In an embodiment where the composition comprising a microglial rescuing agent is administered to a subject, the dose of a microglial rescuing agent may be from about 0.1 mg/kg to about 500 mg/kg. For example, the dose of a microglial rescuing agent may be about 0.1 mg/kg, about 0.5 mg/kg, about 1 mg/kg, about 5 mg/kg, about 10 mg/kg, about 15 mg/kg, about 20 mg/kg, or about 25 mg/kg. Alternatively, the dose of a microglial rescuing agent may be about 25 mg/kg, about 50 mg/kg, about 75 mg/kg, about 100 mg/kg, about 125 mg/kg, about 150 mg/kg, about 175 mg/kg, about 200 mg/kg, about 225 mg/kg, or about 250 mg/kg. Additionally, the dose of a microglial rescuing agent may be about 300 mg/kg, about 325 mg/kg, about 350 mg/kg, about 375 mg/kg, about 400 mg/kg, about 425 mg/kg, about 450 mg/kg, about 475 mg/kg or about 500 mg/kg.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts. For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

### (c) Components of the Composition

The present disclosure also provides pharmaceutical compositions. The pharmaceutical composition comprises a microglial rescuing agent, as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In each of the embodiments described herein, a composition of the invention may optionally comprise one or more additional drug or therapeutically active agent in addition to the microglial rescuing agent. In some embodiments, the additional drug or therapeutically active agent is used to treat central nervous system diseases, or disorders. Other active agents which can be administered together with a microglial rescuing agent include but are not limited to neurotransmitters, neurotransmitter agonists or antagonists, steroids, corticosteroids (such as prednisone or methyl prednisone) immunomodulating agents (such as beta-interferon), immunosuppressive agents (such as cyclophosphamide or azathioprine), nucleotide analogs, endogenous opioids, or other currently clinically used drugs. In some embodiments, the secondary agent is selected from a corticosteroid, a non-steroidal anti-inflammatory drug (NSAID), an intravenous immunoglobulin, a tyrosine kinase inhibitor, a fusion protein, a monoclonal antibody directed against one or more pro-inflammatory cytokines, a chemotherapeutic agent and a combination thereof. In some embodiments, the secondary agent may be a glucocorticoid, a corticosteroid, a non-steroidal anti-inflammatory drug (NSAID), a phenolic antioxidant, an anti-proliferative drug, a tyrosine kinase inhibitor, an anti IL-5 or an IL5 receptor monoclonal antibody, an anti IL-13 or an anti IL-13 receptor monoclonal antibody, an IL-4 or an IL-4 receptor monoclonal antibody, an anti IgE monoclonal antibody, a monoclonal antibody directed against one or more pro-inflammatory cytokines, a TNF-α inhibitor, a fusion protein, a chemotherapeutic agent or a combination thereof. In some embodiments, the secondary agent is an anti-inflammatory drug. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, curcumin, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, lysofylline, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, mepolizumab, prodrugs thereof, and a combination thereof.

(i) Diluent

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

(ii) Binder

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

(iii) Filler

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

(iv) Buffering Agent

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

(v) pH Modifier

In various embodiments, the excipient may be a pH modifier. By way of non-limiting example, the pH modifying agent may be sodium carbonate, sodium bicarbonate, sodium citrate, citric acid, or phosphoric acid.

(vi) Disintegrant

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

(vii) Dispersant

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

(viii) Excipient

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

(ix) Lubricant

In a further embodiment, the excipient may be a lubricant. Non-limiting examples of suitable lubricants include minerals such as talc or silica; and fats such as vegetable stearin, magnesium stearate, or stearic acid.

(x) Taste-Masking Agent

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

(xi) Flavoring Agent

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

(xii) Coloring Agent

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

### (d) Administration

(i) Dosage Forms

The composition can be formulated into various dosage forms and administered by a number of different means that will deliver a therapeutically effective amount of the active ingredient. Such compositions can be administered orally (e.g. inhalation), parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof. For administration by inhalation, the compounds are delivered in the form of an aerosol spray from pressured container or dispenser which contains a suitable propellant, e.g., a gas such as carbon dioxide, or a nebulizer.

For parenteral administration (including subcutaneous, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols, or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising a microglial rescuing agent is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers, and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of a microglial rescuing agent in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the microglial rescuing agent may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying a microglial rescuing agent may be prepared by any known method of preparing liposomes for drug delivery, such as, for example, detailed in U.S. Pat. Nos. 4,241,046; 4,394,448; 4,529,561; 4,755,388; 4,828,837; 4,925,661; 4,954,345; 4,957,735; 5,043,164; 5,064,655; 5,077,211; and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of the microglial rescuing agent, concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The microglial rescuing agent may be encapsulated in a microemulsion by any method generally known in the art.

In yet another embodiment, a microglial rescuing agent may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

### (II) Methods

The present disclosure encompasses a method of modulating microglial activity in a subject or in a sample, the method generally comprising contacting the subject or sample with a composition comprising an effective amount of a microglial rescuing agent. In another aspect, the present disclosure encompasses a method of modulating microglial metabolism in a subject in need thereof, the method generally comprising administering to the subject a composition comprising a therapeutically effective amount of a microglial rescuing agent. In yet another aspect, the present disclosure provides a composition comprising of a microglial rescuing agent for use in vitro, in vivo, or ex vivo. In some embodiments, the present invention provides a method of treating microglial dysfunction in a subject having a microglial dysfunction-associated neurodegenerative disease comprising administering to a subject a therapeutically effective amount of a microglial rescuing agent. One aspect of the present disclosure provides for a treatment of a subject with a neurodegenerative disease (e.g., AD) with creatine, a creatine analog, or Dectin-1 agonist as a treatment to enhance microglial responses by sustaining cell metabolism in individuals with single nucleotide polymorphisms (SNPs) or other mutations affecting microglial functions. Suitable compositions comprising a microglial rescuing agent are disclosed herein, for instance those described in Section I.

Provided is a process of treating a neurological disease, disorder, or condition associated with microglial dysfunction in a subject in need administration of a therapeutically effective amount of a microglial rescuing agent, so as to enhance microglial function, inhibit a neurological disease, disorder, or condition associated with microglial dysfunction, slow the progress of a neurological disease, disorder, or condition associated with microglial dysfunction, or limit the development of a neurological disease, disorder, or condition associated with microglial dysfunction.

There has been an every growing expansion of understanding of the involvement of microglia in central nervous system (CNS) disorders. A host of new molecular tools and mouse models of disease are increasingly implicating this enigmatic type of nervous system cell as a key player in conditions ranging from neurodevelopmental disorders such as autism to neurodegenerative disorders such as Alzheimer's disease and chronic pain. Contemporaneously, diverse roles are emerging for microglia in the healthy brain, from sculpting developing neuronal circuits to guiding learning-associated plasticity. The term “glial cell”, as used herein refers to connective tissue cells of the central nervous system providing structural and functional support to the neuronal cells of the central nervous system, including, for example, in the form of providing nutrition and homeostasis and/or by participation in signal transmission in the nervous system. Glial cells include, but are not limited to, astrocytes (also referred to herein as astroglial cells), microglia, and oligodendrocytes.

The term “microglial cell” or “microglia”, as used herein, refers to a class of glial cells involved in the mediation of an immune response within the central nervous system by acting as macrophages. Microglial cells are capable of producing exosomes, and further include different forms of microglial cells, including amoeboid microglial cells, ramified microglial cells and reactive microglial cells. Microglial cells include reactive microglia, which are defined as quiescent ramified microglia that transform into a reactive, macrophage-like state and accumulate at sites of brain injury and inflammation to assist in tissue repair and neural regeneration.

One aspect of the present disclosure provides for a treatment of a subject with a microglial-dysfunction associated disease or disorder. The microglial-dysfunction associated disease or disorder may be any central nervous system disease or disorder in which disrupted microglial function contributes to pathology or symptoms. In non-limiting examples, microglial-dysfunction associated diseases and disorders include Alzheimer's disease, Parkinson's disease, Nasu-Hakola disease, prion diseases, multiple sclerosis, HIV-dementia, amyotrophic lateral sclerosis (ALS), frontal temporal dementia, neuropathic pain, and autism spectrum disorders. For example, microglial-dysfunction associated diseases and disorders include those described in Salter and Stevens, Nature Medicine volume 23, pages 1018-1027 (2017), the description of which is incorporated herein by reference. In some embodiments, the microglial-dysfunction associated disease or disorder is AD. In one aspect, the microglial-dysfunction associated disease or disorder is associated with mutations in TREM2 or ApoE.

As shown herein, a subject with a neurodegenerative disease (e.g., AD) or a microglial-dysfunction associated disease in a subject with ApoE or TREM2 variants can be treated with a microglial rescuing agent (optionally in combination with conventional treatments). It has been shown that creatine and analogs can provide improved neuroprotection in subjects with ApoE or TREM2 variant compared to a subject with the same disease without the ApoE or TREM2 variant.

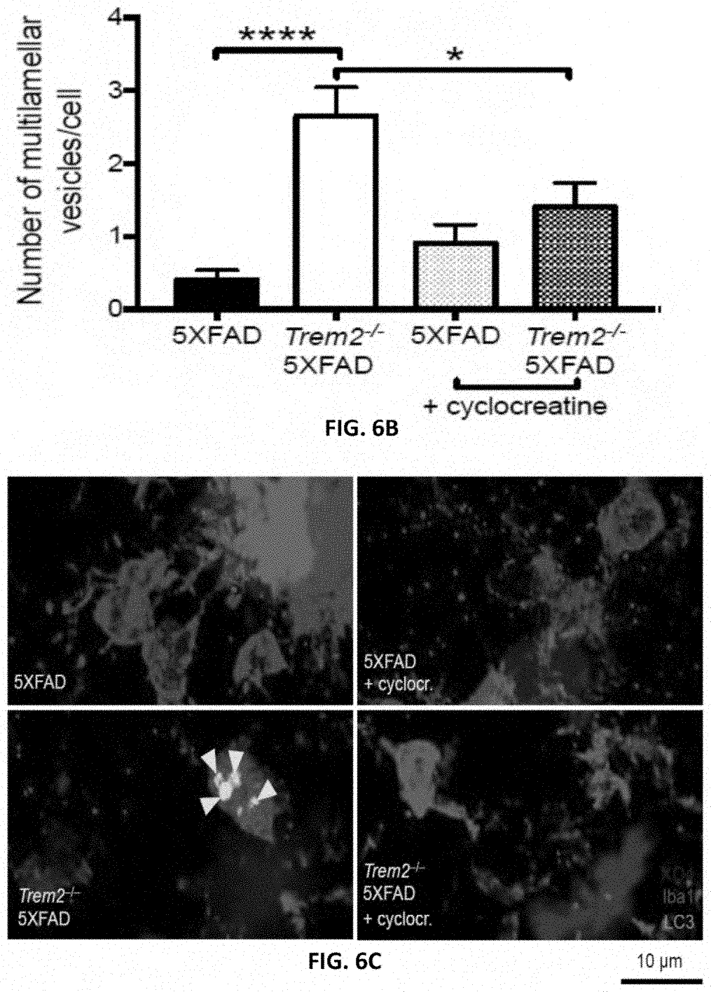

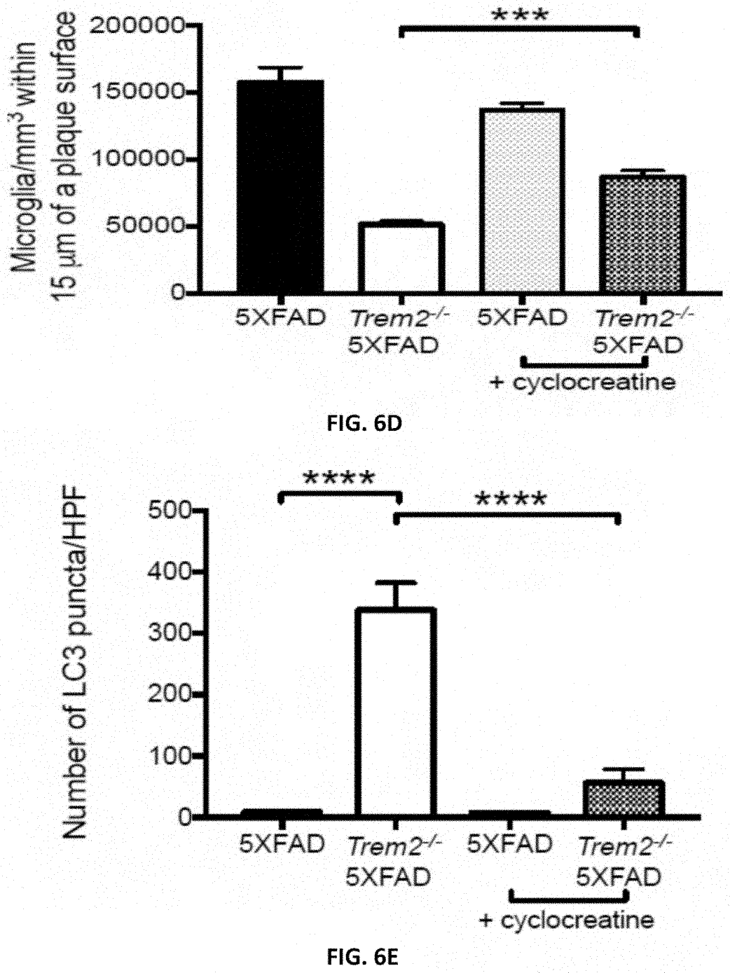

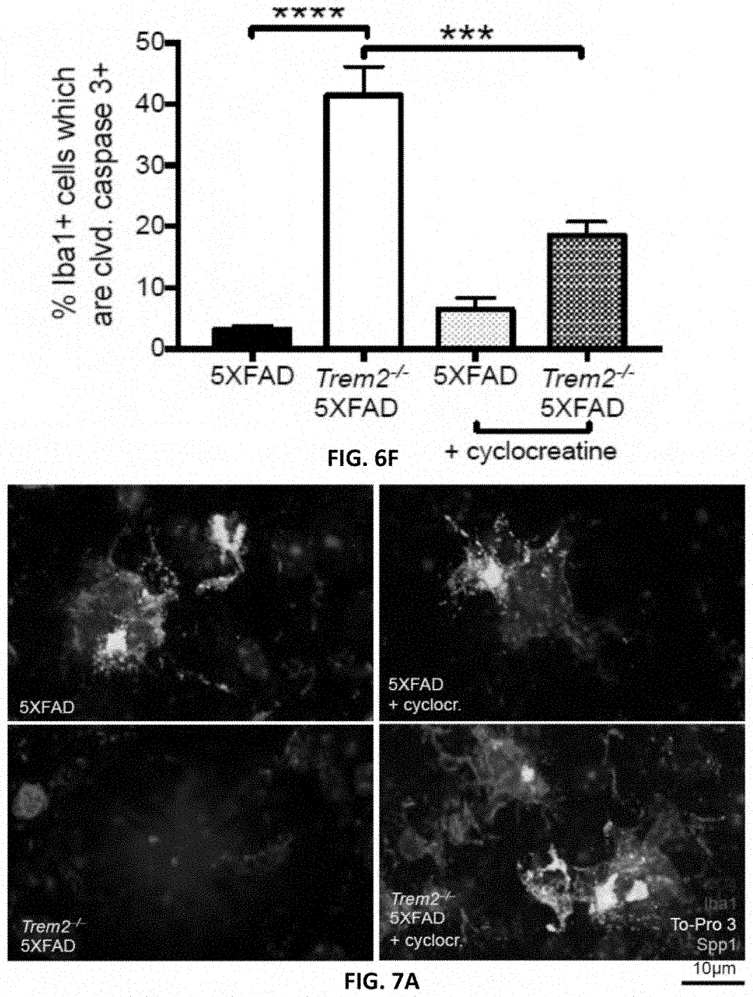

Surprisingly, the present disclosure provides for the identification of a specific subset of patients with mutations in ApoE or TREM2 as having an improved response to a microglial rescuing agent. For example, FIG. 6 presents data showing that the creatine treatment in TREM2 knock out AD mouse model works surprisingly or unexpectedly better than in the AD mouse model without the TREM2 knock out.

Elevated risk of developing Alzheimer's disease (AD) is associated with hypomorphic variants of TREM2, a surface receptor required for microglial responses to neurodegeneration, including proliferation, survival, clustering and phagocytosis. How TREM2 promotes such diverse responses is unknown. Here, We find that microglia in AD patients carrying TREM2 risk variants and TREM2-deficient mice with AD-like pathology have abundant autophagic vesicles, as do TREM2-deficient macrophages under growth factor limitation or ER stress. Combined metabolomics and RNA-seq linked this anomalous autophagy to defective mTOR signaling, which affects ATP levels and biosynthetic pathways. Metabolic derailment and autophagy were offset in vitro through Dectin-1, a receptor that elicits TREM2-like intracellular signals, and cyclocreatine, a creatine analog that can supply ATP. Dietary cyclocreatine markedly tempered autophagy, restored microglial clustering around plaques, and decreased plaque-adjacent neuronal dystrophy in TREM2-deficient mice with amyloid-β pathology. Thus, TREM2 enables microglial responses during AD by sustaining cellular energetic and biosynthetic metabolism.

Several creatine analogs have been used in vitro and in vivo to supplement deficiency in TREM2, which can play an anti-inflammatory role in the pathogenesis of Alzheimer's. Microglia of mice and humans deficient in Trem-2 undergo increased autophagy in response to stresses such as plaques associated with AD. Treatment of Trem-2 deficient macrophages with nicotinamide mononucleotide, cyclocreatine, and phosphocreatine was able to rescue metabolic activity and prevent autophagy and cell death. In addition, excessive neuronal damage was reversed in vivo when the compounds were administered to Trem-2 deficient 5XFAD mice. The invention can be utilized to decrease the risk/severity of AD in patients carrying mutations in ApoE or Trem2 that limit the function of microglial cells.

According to an aspect of the invention a pharmaceutical composition comprising a microglial rescuing agent is used for modulating microglial activity. The method generally comprises contacting a microglia with a pharmaceutical composition comprising a microglial rescuing agent. In some embodiments, the method comprising contacting a microglia in vivo by administering a pharmaceutical composition comprising a microglial rescuing agent. Microglial activity can be measured by cell viability, mTOR signaling, presence/absence of autophagy, Syk signaling, PI3-K signaling, microgliosis, microglial clustering, neurite dystrophy, and microglial metabolism. Standard techniques and assays may be used to measure microglial activity including those described in the examples below. In some embodiments, microglial activity is measured by cell viability, wherein increased cell viability indicates increased microglial activity. In some embodiments, microglial activity is measured by mTOR signaling, wherein increased mTOR signaling indicates increased microglial activity. In some embodiments, microglial activity is measured by the presence of autophagy, wherein decreased autophagy indicates increased microglial activity. In some embodiments, contacting a microglia with a pharmaceutical composition comprising a microglial rescuing agent results in increased microglial activity relative to an untreated control.

In certain aspects, a therapeutically effective amount of a composition of the invention may be administered to a subject. Methods described herein are generally performed on a subject in need thereof. A subject in need of the therapeutic methods described herein can be a subject having, diagnosed with, suspected of having, or at risk for developing a neurological disease, disorder, or condition associated with microglial dysfunction. A determination of the need for treatment will typically be assessed by a history and physical exam consistent with the disease or condition at issue. Diagnosis of the various conditions treatable by the methods described herein is within the skill of the art. Administration is performed using standard effective techniques, including peripherally (i.e. not by administration into the central nervous system) or locally to the central nervous system. Peripheral administration includes but is not limited to oral, inhalation, intravenous, intraperitoneal, intra-articular, subcutaneous, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation. The route of administration may be dictated by the disease or condition to be treated. For example, if the disease or condition is COPD or IPF, the composition may be administered via inhalation. Alternatively, is the disease or condition is osteoarthritis, the composition may be administered via intra-articular invention. It is within the skill of one in the art, to determine the route of administration based on the disease or condition to be treated. In a specific embodiment, a composition of the invention is administered orally.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents, and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

For therapeutic applications, a therapeutically effective amount of a composition of the invention is administered to a subject. A “therapeutically effective amount” is an amount of the therapeutic composition sufficient to produce a measurable response. In various embodiments, an effective amount of a microglial rescuing agent described herein can substantially enhance microglial function, inhibit a neurological disease, disorder, or condition associated with microglial dysfunction, slow the progress of a neurological disease, disorder, or condition associated with microglial dysfunction, or limit the development of a neurological disease, disorder, or condition associated with microglial dysfunction. Actual dosage levels of active ingredients in a therapeutic composition of the invention can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, age, the age-related disease or condition, the degenerative disease, the function-decreasing disorder, the symptoms, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

The frequency of dosing may be daily or once, twice, three times, or more per day, per week or per month, as needed as to effectively treat the symptoms. The timing of administration of the treatment relative to the disease itself and duration of treatment will be determined by the circumstances surrounding the case. In some embodiments, treatment begins immediately, such as at the site of the injury as administered by emergency medical personnel. In some embodiments, treatment begins in a hospital or clinic itself, or at a later time after discharge from the hospital or after being seen in an outpatient clinic. Depending upon the embodiment, duration of treatment ranges from a single dose administered on a one-time basis to a life-long course of therapeutic treatments.

Typical dosage levels can be determined and optimized using standard clinical techniques and will be dependent on the mode of administration.

A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

### Definitions

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As used herein, the following definitions shall apply unless otherwise indicated. For purposes of this invention, the chemical elements are identified in accordance with the Periodic Table of the Elements, CAS version, and the Handbook of Chemistry and Physics, 75^{th}Ed. 1994. Additionally, general principles of organic chemistry are described in “Organic Chemistry,” Thomas Sorrell, University Science Books, Sausalito: 1999, and “March's

Advanced Organic Chemistry,” 5^{th} Ed., Smith, M. B. and March, J., eds. John Wiley & Sons, New York: 2001, the entire contents of which are hereby incorporated by reference.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction

As shown herein, the present disclosure provides for the use of a microglial rescuing agent that can increase microglial energetic metabolism in patients with mutations in genes which increase the odds for the development of Alzheimer's disease by effecting microglial function i.e. ApoE, Trem2. Microglial rescuing agents such as NMN, cyclocreatine, and phosphocreatine have been used in vitro and in the case of cyclocreatine in vivo to supplement Trem2-deficient cells or mice with AD-like pathology (5XFAD mice).

As shown herein, microglia in Trem2-deficient mice and humans carrying disease associated SNPs in TREM2 are less energetically competent and exhibit increased autophagy in response to stress such as the pathologic state associated with Alzheimer's disease. Supplementation of media with wild-type and Trem2-deficient bone marrow derived macrophages with NMN, cyclocreatine, and phosphocreatine were able to rescue Trem2-deficient cells metabolic capacity and prevent autophagy and cell death. In in vivo studies in SXFAD and Trem2-deficient SXFAD mice We show that We can increase microgliosis, microglial clustering around plaques, decrease microglial autophagy, decrease microglial cell death, and subsequently decrease neuronal dystrophy around plaques.

As shown herein, LC3 positive puncta was observed in microglia in individuals carrying disease associated SNPs in TREM2 and that these puncta are reminiscent of the LC3 puncta in microglia in 5XFAD mice. Trem2-deficient and ApoE-deficient mice have similar microglial response to plaque deposition. Cyclocreatine treatment of Trem2-deficient 5XFAD prevented the formation of autophagic vesicles and cell death while reversing excessive neuronal damage. This invention could treat and potentially normalize the risk for the development of Alzheimer's disease and limit disease severity in individuals carrying mutations which compromise microglial responsiveness to amyloid β plaques in Alzheimer's. Such genes include ApoE and Trem2.

Electron and confocal microscopy was used to analyze microglia from 5XFAD mice, which develop AR accumulation that mimics AD pathology due to the expression of mutant APP and PS1 under neural-specific elements of the mouse Thyl promoter. Microglia from 5XFAD mice lacking TREM2 had many more autophagic vesicles than did microglia in SXFAD mice. This observation was replicated in humans, as microglia in AD patients carrying TREM2 risk variants also had more autophagic vesicles than did microglia in AD patients with the common TREM2 variant. Autophagy is an intracellular degradation pathway essential for cellular and energy homeostasis. It provides a mechanism for the elimination of misfolded proteins and damaged organelles and compensates for nutrient deprivation during cell starvation through recycling of cytosolic components. Because autophagy is partially regulated by mammalian target of rapamycin (mTOR)-dependent pathways (Saxton and Sabatini, 2017), the impact of TREM2-deficiency on mTOR activation was assessed and found that, indeed, anomalous autophagy reflected defective activation of mTOR signaling. Similarly, enhanced autophagy was observed in TREM2-deficient macrophages in vitro, which was further amplified by growth-factor limitation or endoplasmic reticulum (ER) stress; this provided a model system for probing biochemical and metabolic pathways in microglia during Aβ accumulation. Combined metabolomics, RNA sequencing (RNA-seq), and system analyses of TREM2-deficient macrophages confirmed the impairment of mTOR activation, energetic pathways, ATP levels, and biosynthetic pathways. Thus, TREM2 sustains cell energetic and biosynthetic metabolism through mTOR signaling. Metabolic derailment and autophagy were offset in vitro through activation of Dectin-1, a surface receptor that triggers a signaling pathway similar to that of TREM2-DAP12 (Dambuza and Brown, 2015). Metabolic abnormalities were also rescued by incubating cells with the creatine analog 1-carboxymethyl-2-iminoimidazolidine (cyclocreatine), which can passively cross membranes and, upon phosphorylation by creatine kinase, generate a supply of ATP for energy demands independent of the TREM2-mTOR axis. Remarkably, dietary administration of cyclocreatine in 5XFAD mice lacking TREM2 prevented microglial autophagy, enhanced microglia numbers and clustering around Aβ plaques, and mitigated plaque-associated neurite dystrophy. This provides proof of principle that strategies aimed at sustaining basic microglial metabolism may be promising for treatment of AD and other neurodegenerative diseases associated with microglial dysfunction.

### Example 1: Defect in TREM2 Elicits Autophagy In Vivo in the 5XFAD Mouse Model and AD Patients

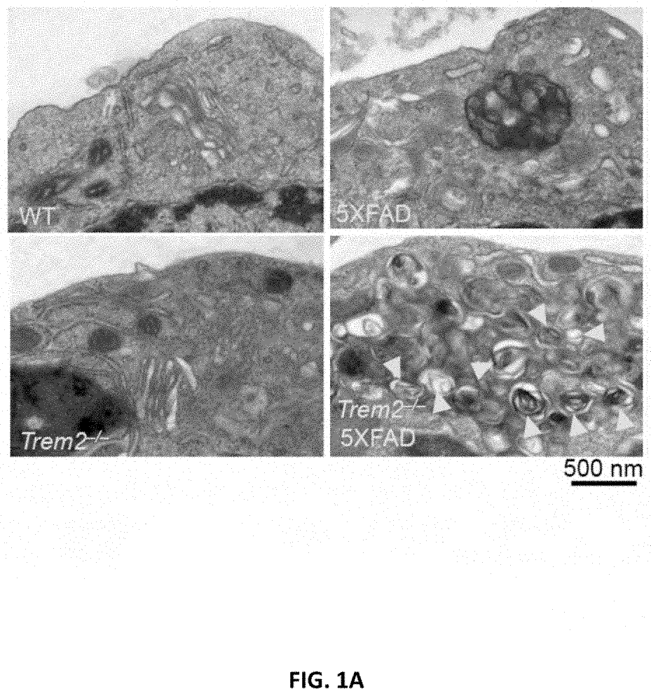

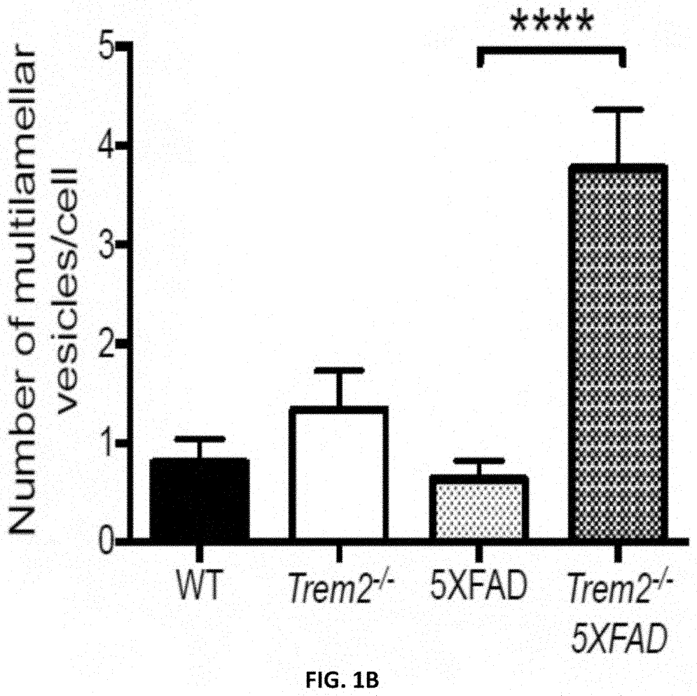

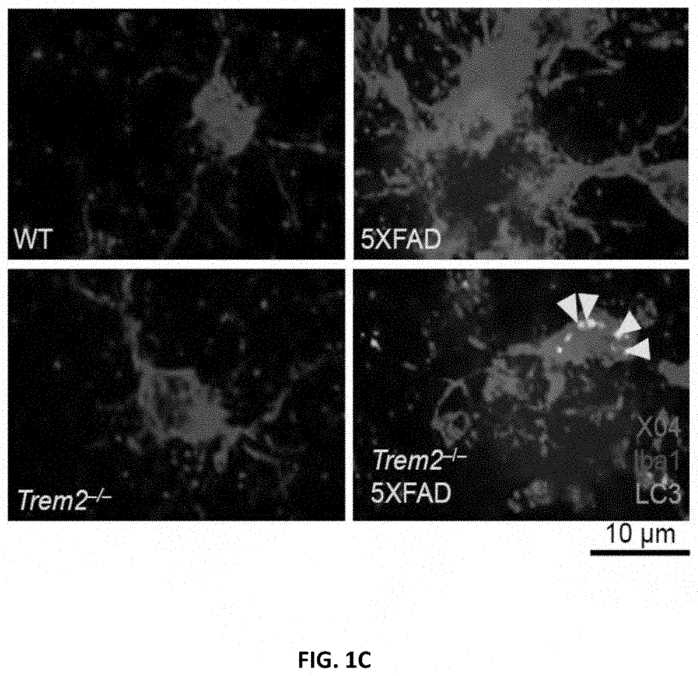

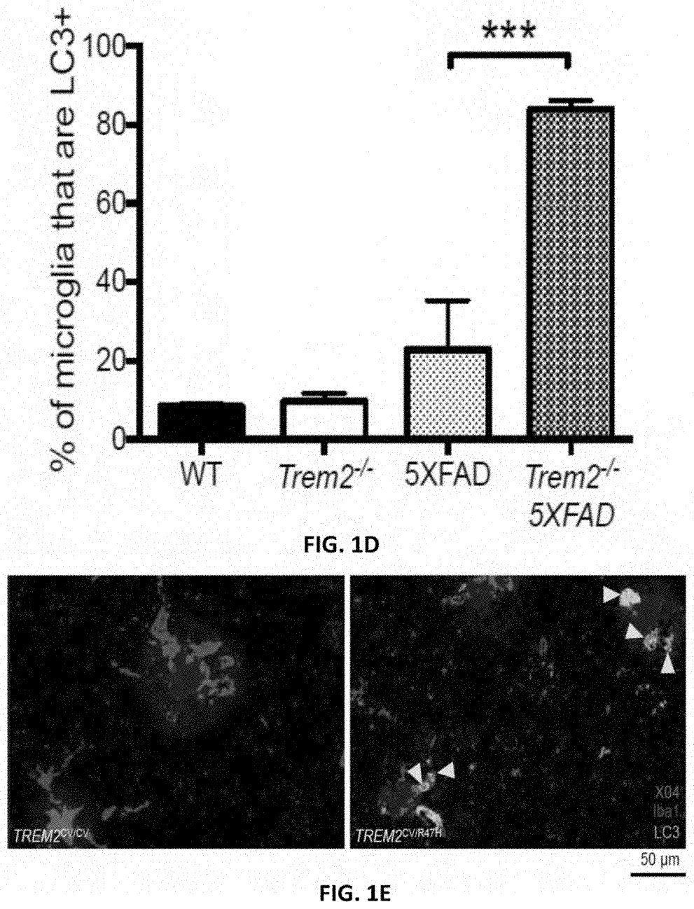

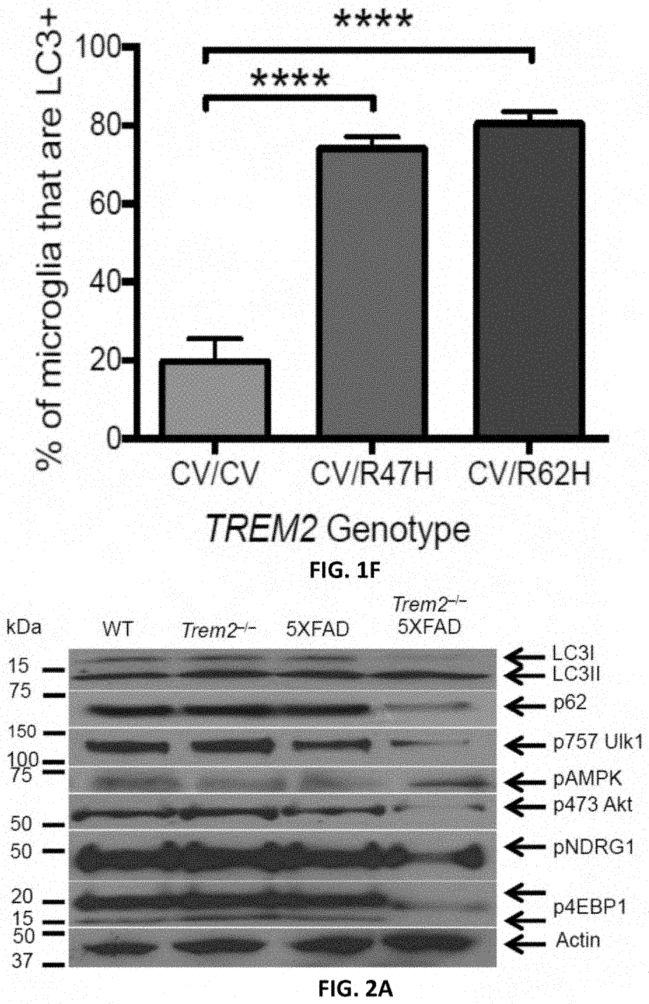

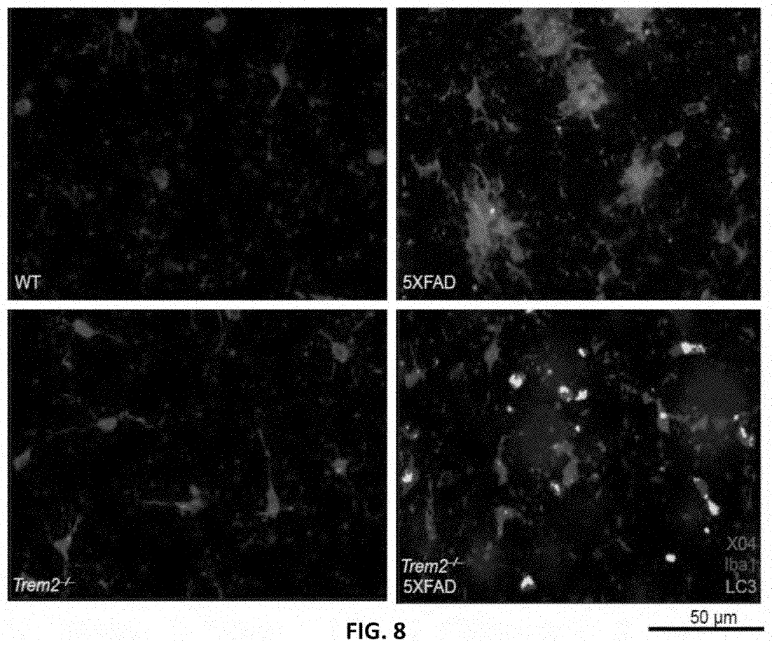

To determine the impact of TREM2 deficiency on microglia function, We directly examined the structure of microglia from the 5XFAD mouse model of AD by transmission electron microscopy (TEM). Strikingly, microglia from Trem2^{-/-} 5XFAD mice contained abundant multivesicular/multilamellar structures suggestive of autophagosomes, which were largely absent in microglia from 5XFAD, Trem2^{-/-} or wild-type (WT) mice (FIG. 1A, FIG. 1B). To determine whether these structures reflected ongoing autophagy in situ, We examined brain sections by confocal microscopy for the presence of LC3⁺ puncta, which denote autophagosomes decorated by lipidated LC3II. Many large LC3⁺ puncta were evident in microglia in Trem2^{-/-} 5XFAD mice, whereas LC3⁺ puncta were sparse in microglia in WT, Trem2^{-/-}, and 5XFAD mice (FIG. 1C, FIG. 1D and FIG. 8). Remarkably, these observations were translatable to human disease. We observed dramatically more LC3⁺ microglia in post-mortem brain sections from both R47H and R62H heterozygous AD patients than in those from case-matched AD patients homozygous for the common TREM2 variant (FIG. 1E, FIG. 1F, Table 1). Taken together, these data suggest that autophagic-like vesicles accumulate in the microglia of TREM2-deficient mice and humans with TREM2 risk alleles during the development of AD.

TABLE 1
Characteristics of Human Tissue Donors (Related to FIG. 1)

| | Age | Braak | CDR (Est. | | TREM2 |
|---|---|---|---|---|---|
| Sex | (yrs) | Stage | at TOD) | CERAD | Status |
| TREM2 Variant | | | | | |
| Carrier | | | | | |
| Female | 93.98 | N.A. | 3 | N.D. | R47H/CV |
| Male | 74.85 | V | 2 | Definite | R47H/CV |
| Male | 83.75 | N.A. | 3 | N.D. | R47H/CV |
| Male | 90.64 | V | 3 | Definite | R47H/CV |
| Female | 88.22 | IV | 3 | Definite | R47H/CV |
| Male | 85.64 | V | 3 | Definite | R47H/CV |
| Male | 78.23 | V | 3 | Definite | R47H/CV |
| Female | 83.16 | N.A. | 3 | N.D. | R62H/CV |
| Female | 89.31 | VI | 3 | Definite | R62H/CV |
| Female | 89.37 | N.A. | 3 | N.D. | R62H/CV |
| Female | 78.98 | N.A. | 3 | N.D. | R62H/CV |
| Case-Matched | | | | | |
| Control | | | | | |
| Female | 95.73 | VI | 3 | Definite | CV/CV |
| Male | 71.6 | VI | 3 | Definite | CV/CV |
| Male | 87.57 | V | 3 | Definite | CV/CV |
| Male | 91.08 | V | 1 | Definite | CV/CV |
| Female | 84.47 | V | 3 | Definite | CV/CV |
| Male | 80.97 | VI | 3 | Definite | CV/CV |
| Female | 85.43 | N.A. | 3 | N.D. | CV/CV |
| Female | 81.26 | VI | 3 | Definite | CV/CV |

### Example 2: TREM 2 Deficiency Impairs mTOR Signaling and Enhances AMPK Activation in Microglia

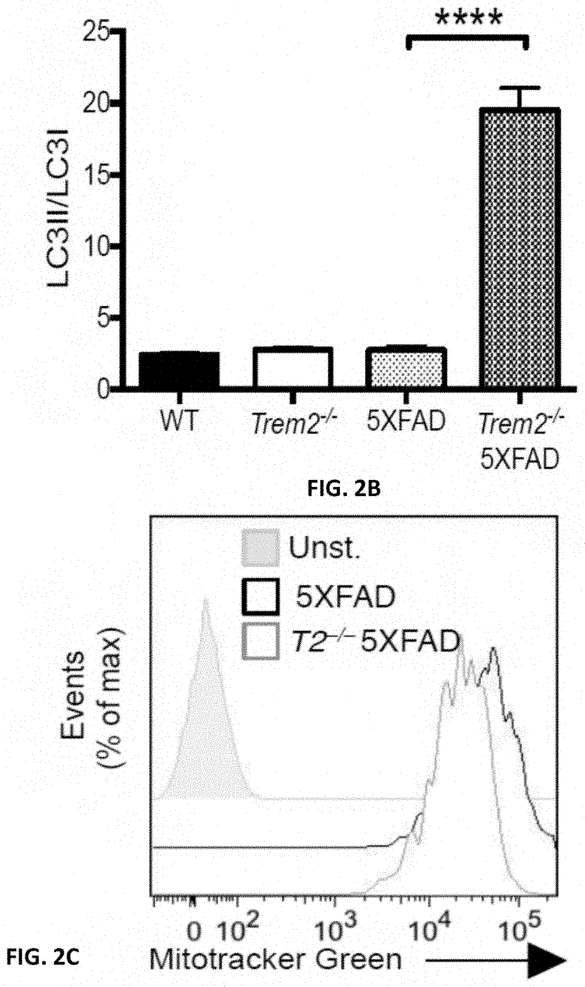

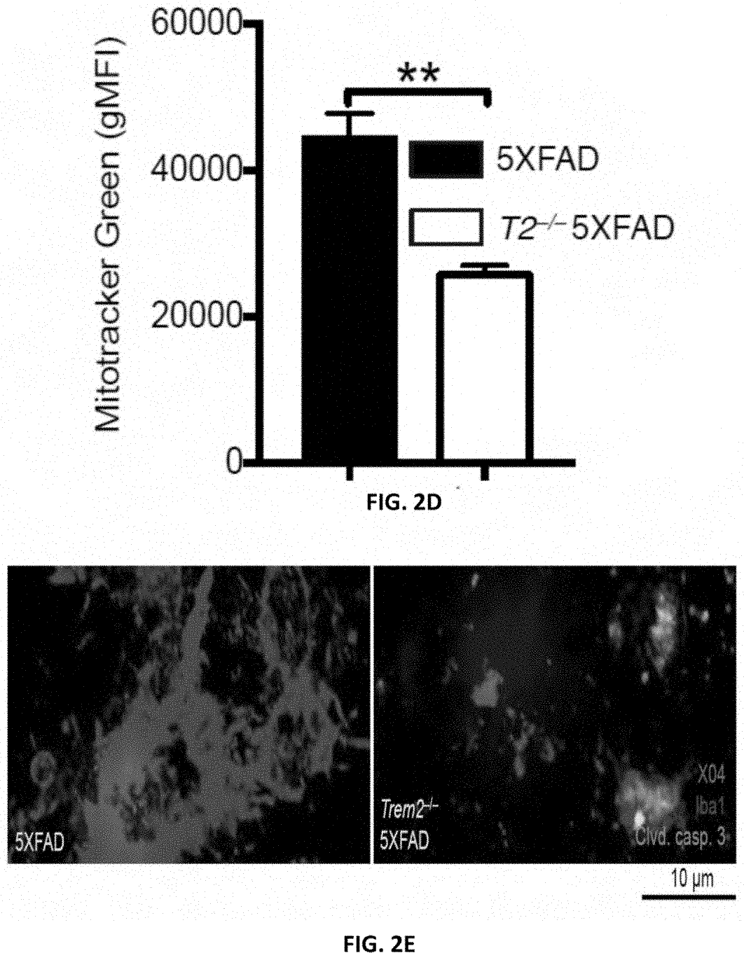

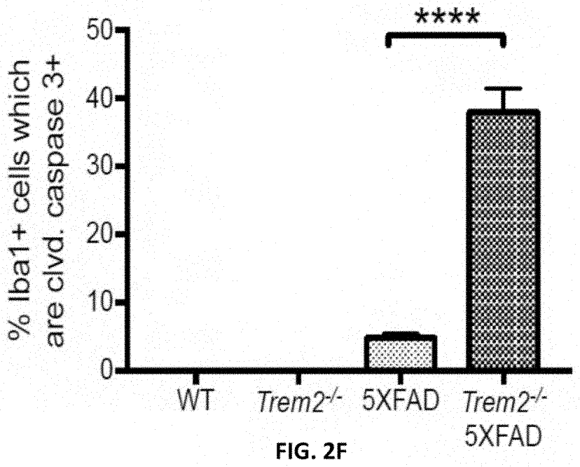

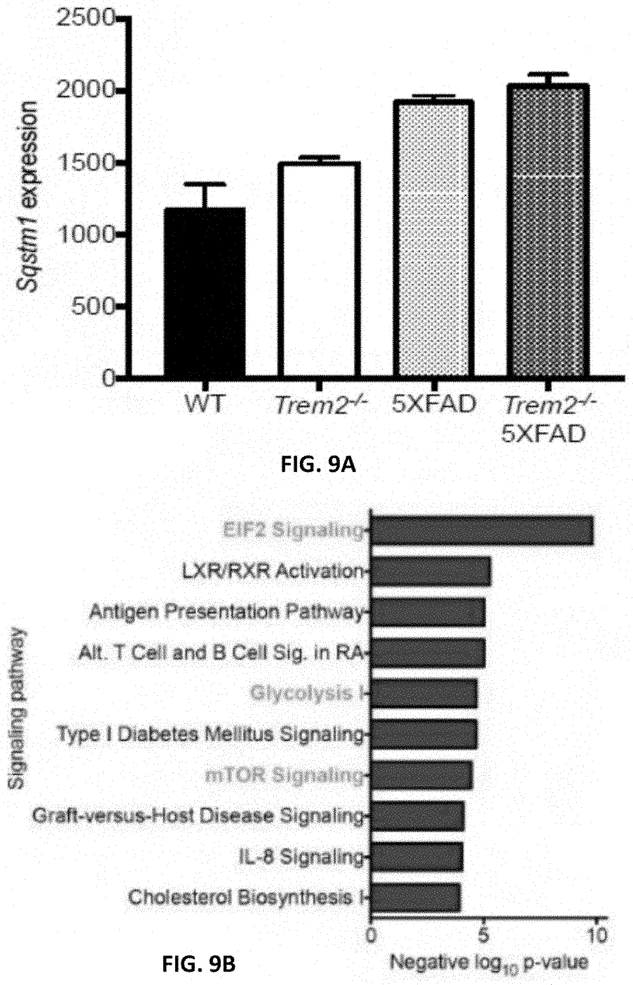

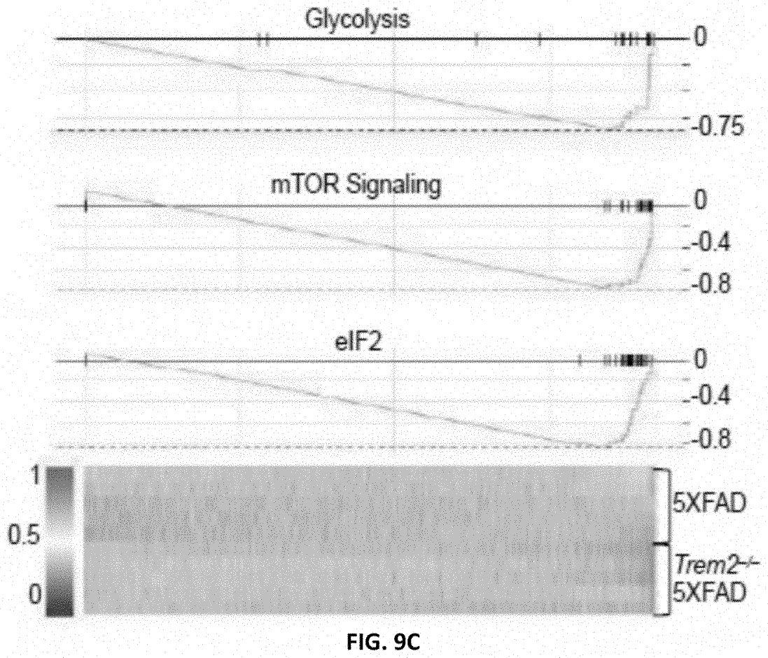

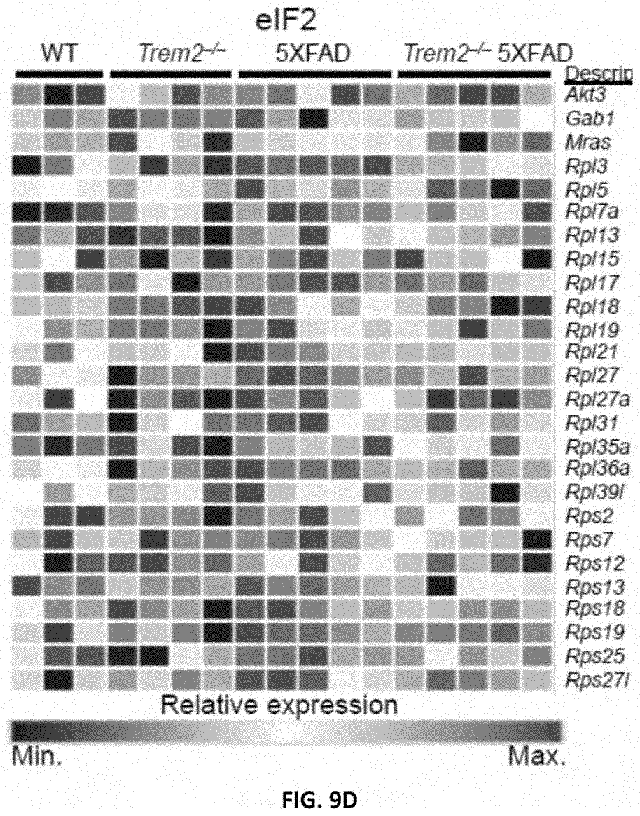

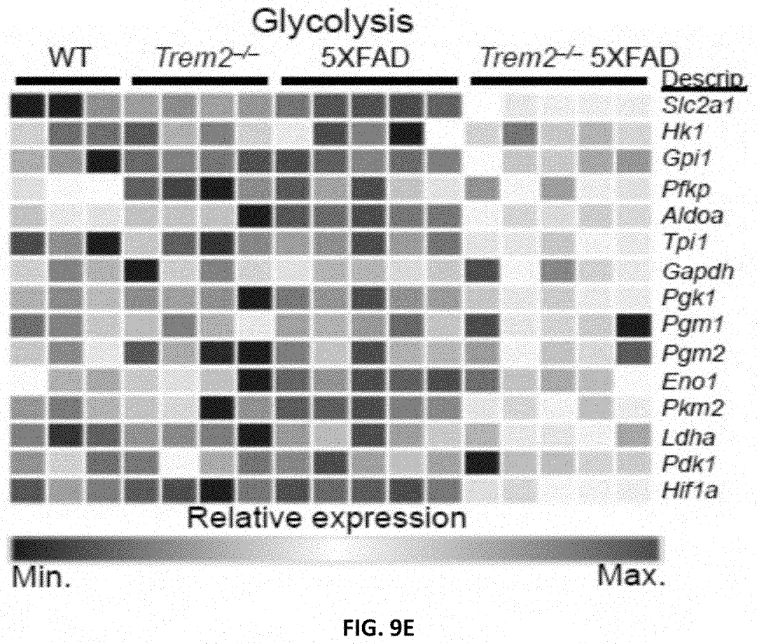

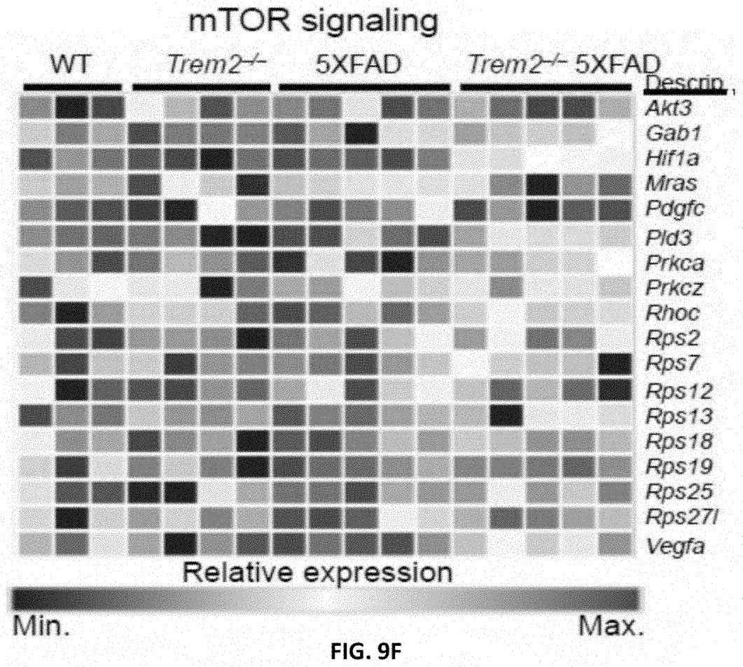

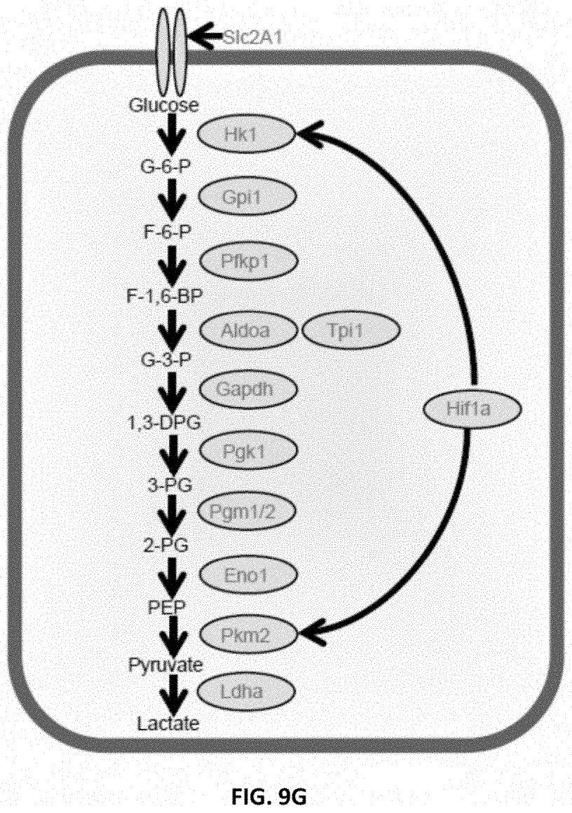

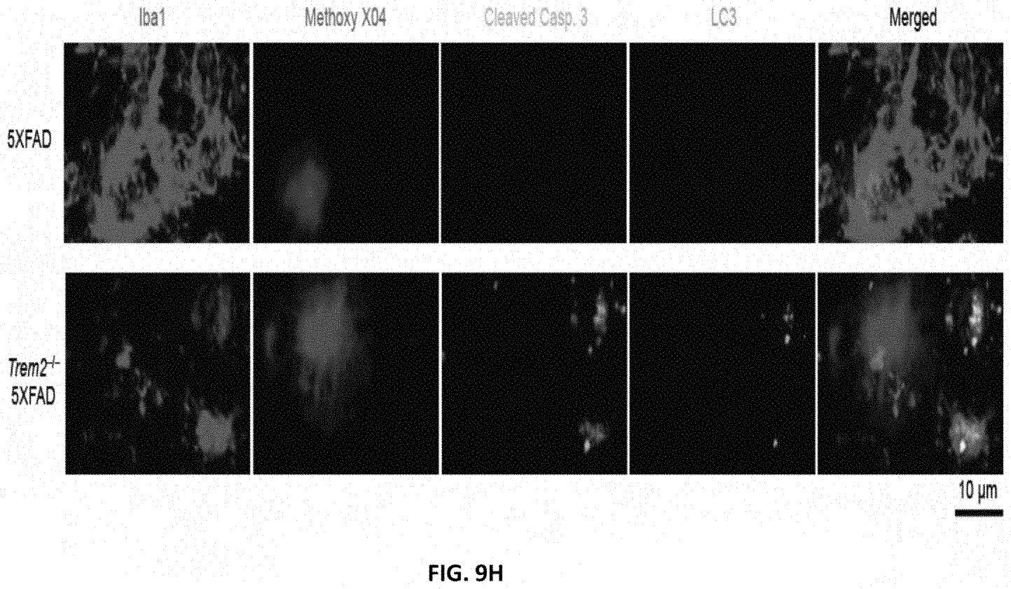

To corroborate the association between TREM2 deficiency and increased autophagy, We performed biochemical analyses on sorted microglia ex vivo. The ratio of lipidated LC3II to non-lipidated LC3I was markedly higher in microglia from Trem2^{-/-} 5XFAD mice than in microglia from 5XFAD mice, consistent with the increased number of autophagic vesicles in TREM2-deficient microglia. To determine whether the increase in autophagosomes was due to activation of autophagy or blockade of lysosomal degradation, We measured protein and mRNA levels of p62, an autophagy cargo protein that is digested by lysosomal enzymes. The amount of p62 protein was lower in microglia from Trem2^{-/-} 5XFAD mice than in microglia from 5XFAD mice. This difference was unrelated to transcription, as p62 (Sqstm1) mRNA levels were similar. Thus, TREM2 deficiency results in bona fide autophagy (FIG. 2A, FIG. 2B and FIG. 9A).

Why is autophagic flux amplified in 5XFAD mice lacking TREM2? Autophagy often reflects an adaptive response to stress that can occur when cells cannot satisfy increased demands for energy and protein synthesis. Since the serine/threonine kinase target of rapamycin (mTOR) has a crucial role in stimulating both energetic and anabolic metabolism, cell growth and proliferation (Laplante and Sabatini, 2012), We hypothesized that the autophagy observed in microglia in Trem2^{-/-} 5XFAD mice might result from a defect in mTOR signaling. mTOR signals through two distinct complexes, mTORC1 and mTORC2. Immunoblotting of sorted microglia from Trem2^{-/-} 5XFAD and 5XFAD mice revealed decreased phosphorylation of 4EBP1, an mTORC1 effector, as well as AKT at serine 473 and NDRG1, both mTORC2 effectors, in the TREM2-deficient microglia (FIG. 2A). Ulk1, a key inducer of autophagy, which is inhibited by mTOR signaling through phosphorylation at serine 757, was less phosphorylated in microglia from Trem2^{-/-} 5XFAD mice, consistent with reduced mTOR activation and increased autophagy. Impaired mTOR signaling was associated with phosphorylation of AMP-activated protein kinase (AMPK), a sensor of low energy states (FIG. 2A).

Providing further evidence for defective energetic and anabolic metabolism associated with TREM2 deficiency, microglia from Trem2^{-/-} 5XFAD mice had a lower mitochondrial mass than did microglia from 5XFAD mice (FIG. 2C, FIG. 2D). Furthermore, gene expression microarray analyses of sorted microglia from Trem2^{-/-} 5XFAD and 5XFAD mice revealed decreased expression of genes encoding translation initiation factors, ribosomal proteins, glucose transporters, glycolytic enzymes, as well as the transcription factor HIF1α that controls glycolysis in TREM2-deficient microglia (FIG. 9B-FIG. 9G). Taken together, these data demonstrate that during the development of AD, TREM2 deficiency derails the mTOR pathway, anabolic and energetic metabolism in microglia, which induces compensatory autophagy in both mice and humans.

To determine whether autophagy in TREM2-deficient microglia can effectively compensate for the metabolic defects in vivo and prevent apoptosis, We examined brain sections by confocal microscopy for the presence of cleaved caspase-3, an indicator of apoptosis. We found that cleaved caspase-3 was much more abundant in microglia in Trem2^{-/-} 5XFAD mice than in microglia in 5XFAD mice (FIG. 2E, FIG. 2F). Additionally, the percentage of microglia with colocalization of cleaved caspase-3 and LC3⁺ puncta was higher in Trem2^{-/-} 5XFAD mice than in 5XFAD mice (FIG. 9H, FIG. 9I). Thus, autophagy may not be sufficient to sustain the microglial response to stress, at least at the late time point of disease progression analyzed.

### Example 3: Macrophages Lacking TREM2 Inadequately Signal Through mTOR and Undergo More Autophagy

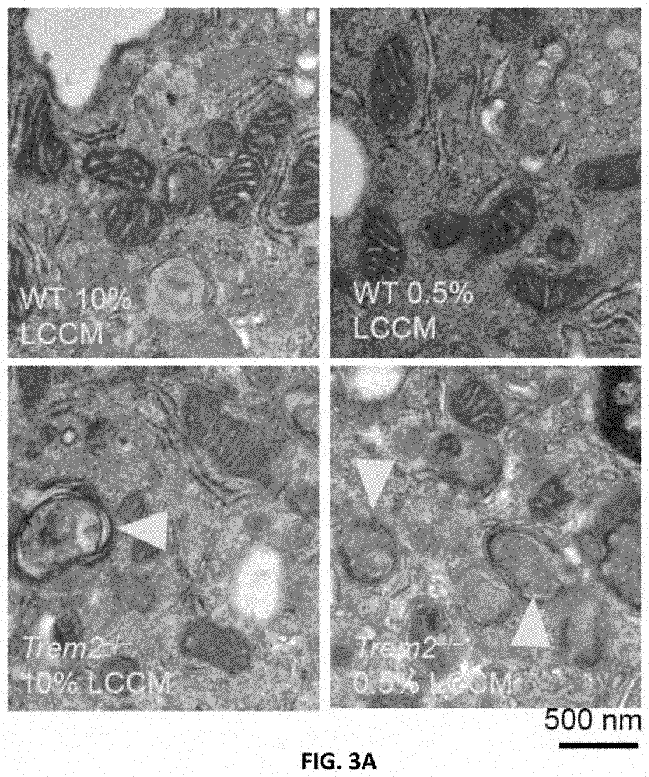

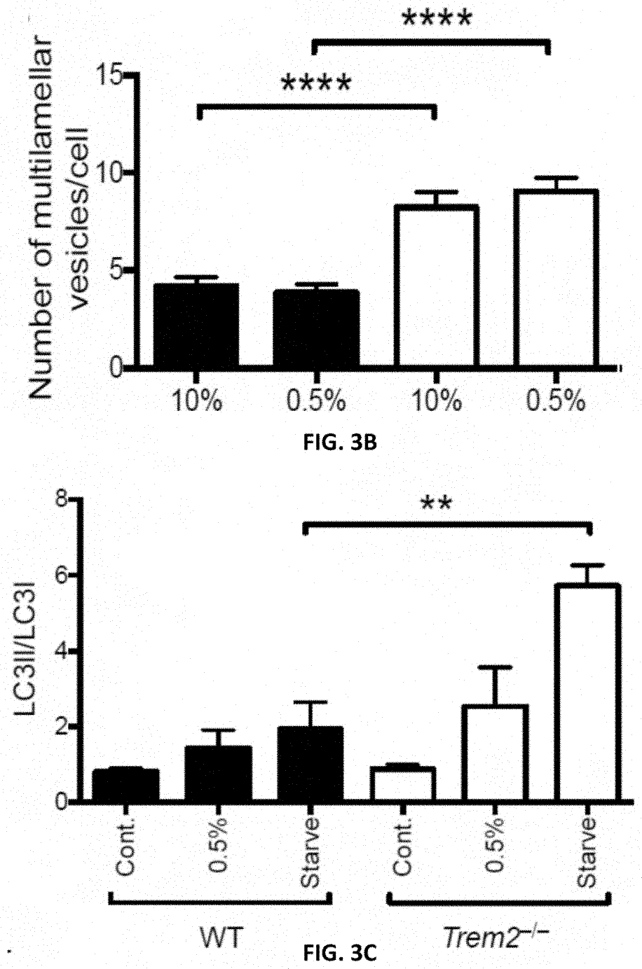

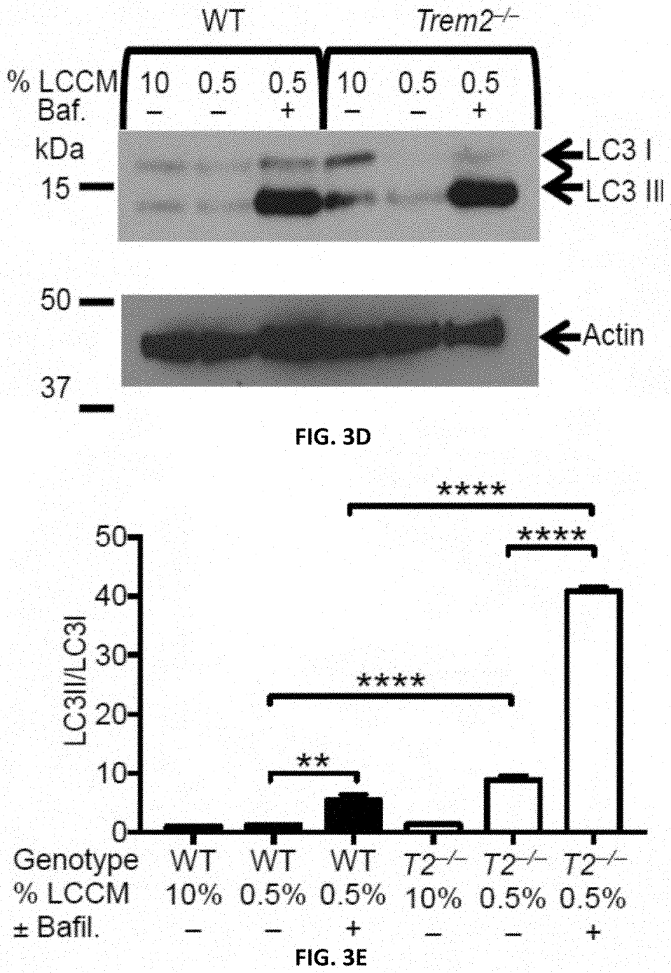

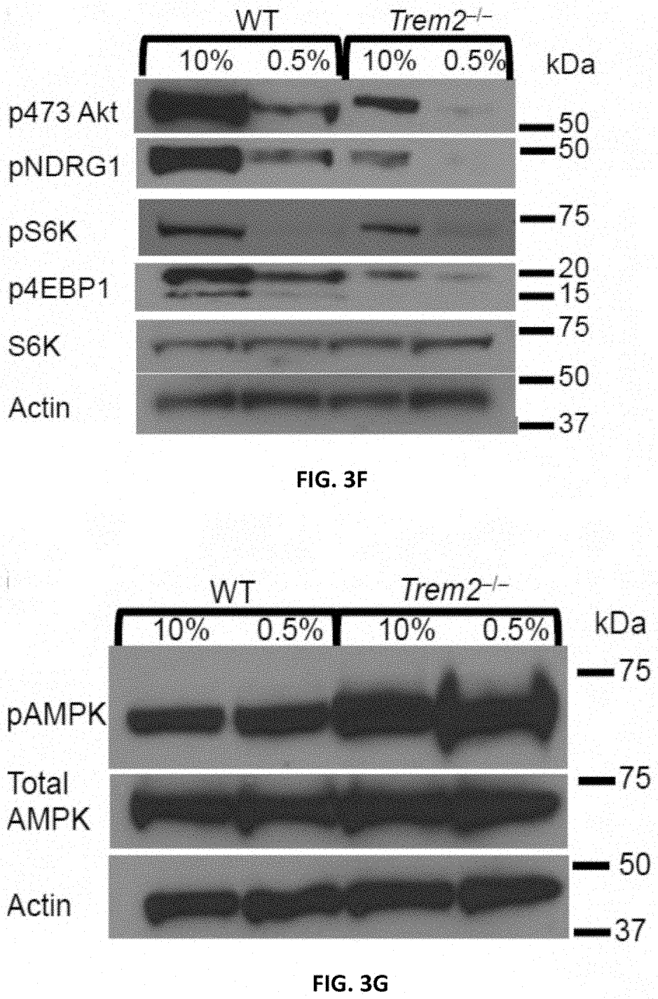

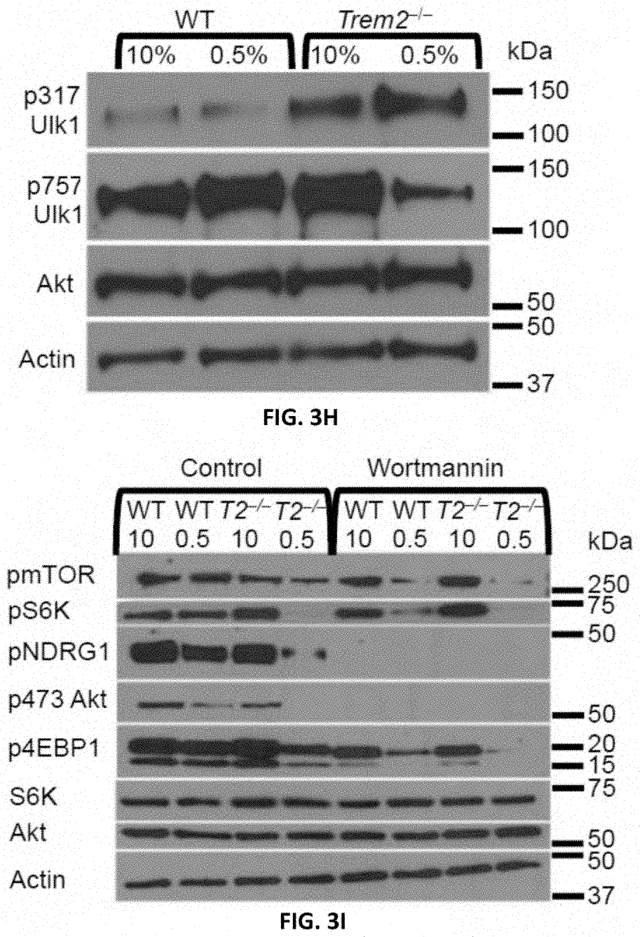

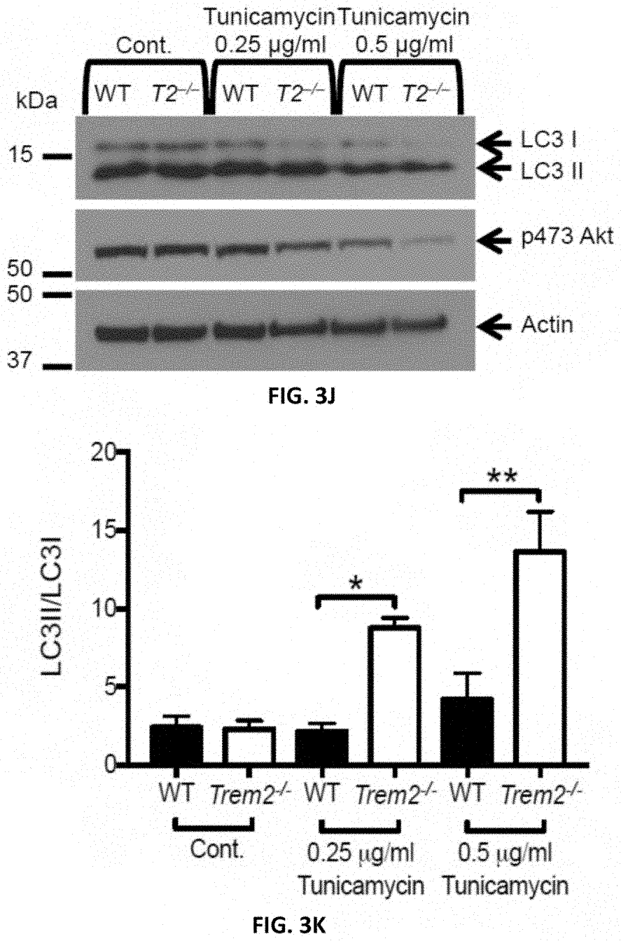

It was then contemplated whether TREM2-deficiency could derail mTOR signaling in bone marrow-derived macrophages (BMDMs) from WT and Trem2^{-/-} mice in vitro. To mimic the metabolic stress that occurs during disease, We used growth factor deprivation. BMDMs were cultured overnight in concentrations of CSF1-containing L cell-conditioned medium (LCCM) ranging from optimal to limiting (10% to 0.5%). Trem2^{-/-} BMDMs contained more autophagic vesicles (FIG. 3A, FIG. 3B) and had a higher LC3II/LC3I ratio than did WT cells when CSF-1 was limiting (FIG. 3C). Addition of the lysosomal inhibitor bafilomycin greatly increased LC3II in Trem2^{-/-} BMDMs, confirming that the increase in autophagosomes was due to increased autophagic flux rather than reduced autophagosome degradation (FIG. 3D, FIG. 3E).

As observed in sorted microglia, autophagy in BMDMs was linked to impaired mTOR signaling. Trem2^{-/-} BMDMs had less phosphorylated 4EBP1, 473S AKT and NDRG1 (FIG. 3F) and more phosphorylated AMPK (FIG. 3G) in both optimal CSF1 and limiting CSF1 than did WT BMDMs. In limiting CSF1, Trem2^{-/-} BMDMs had decreased inhibitory phosphorylation of Ulk1 at serine 757, while activating phosphorylation of Ulk1 at serine 317 increased (FIG. 3H). Thus, lack of TREM2 suppressed mTOR activation and elicited compensatory AMPK and Ulk1 activation and autophagy in BMDMs in response to metabolic stress, very similar to our observations of microglia in 5XFAD mice.

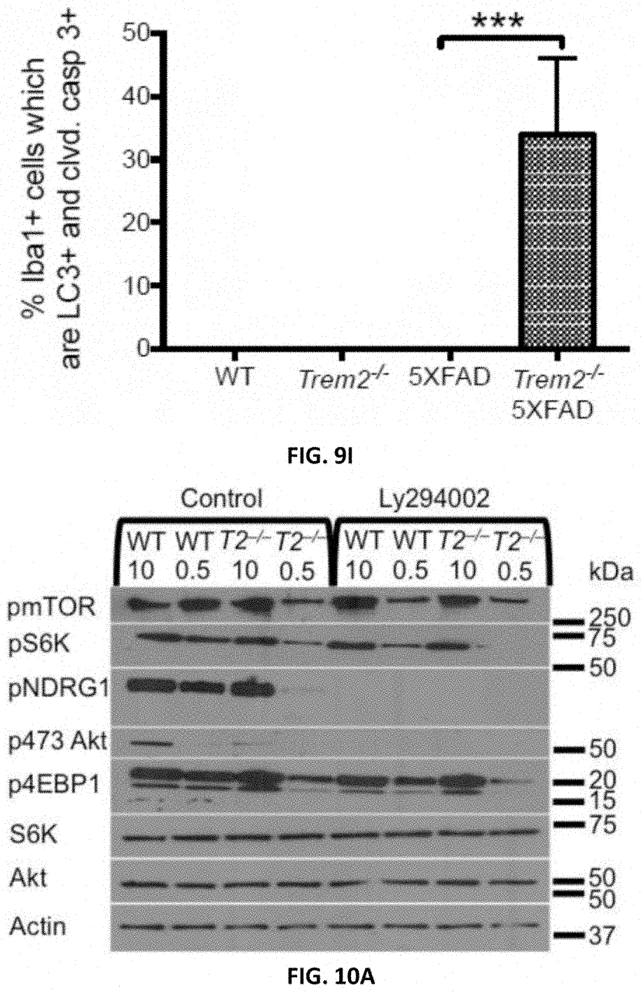

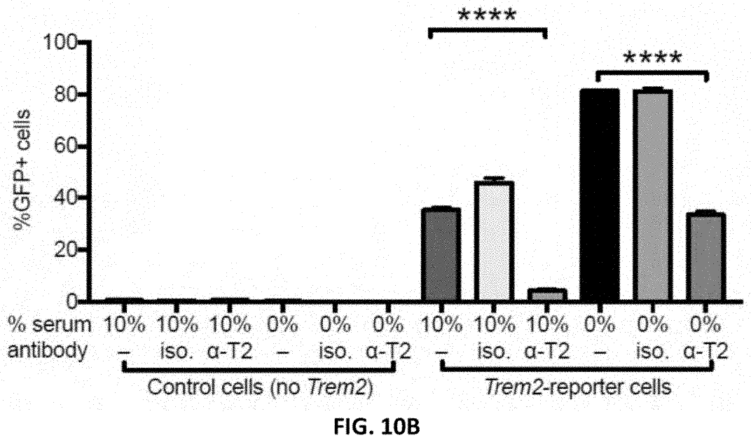

As TREM2 signaling adapters DAP12 and DAP10 have been shown to activate PI3-K, which in turn can activate mTOR, We asked whether enhanced mTOR signaling in WT BMDM compared to Trem2^{-/-} BMDM was dependent on PI3-K. Inhibition of PI3-K with wortmannin or LY294002 caused a major reduction in phosphorylation of mTOR and its downstream targets in WT BMDMs; the residual amount of phosphorylation was similar to that seen in Trem2^{-/-} BMDMs (FIG. 3I and FIG. 10A). In addition to limiting CSF1, other stressors may differentially modulate mTOR signaling and autophagy in WT and Trem2^{-/-} BMDMs. Treatment with tunicamycin, which provokes endoplasmic reticulum (ER) stress, the unfolded protein response and autophagy, induced greater LC3II/LC3I ratios and less Akt S473 phosphorylation in Trem2^{-/-} BMDMs than in WT BMDMs (FIG. 3J, FIG. 3K). Thus, TREM2 deficiency affects cell responses to multiple stressors.

TREM2 signaling may not just have a more pronounced effect on cells under stress conditions, but may actually increase under such conditions. When cultured in media containing 10% FBS, TREM2 reporter cells, which express GFP upon TREM2 engagement, showed some activation (FIG. 10B). However, upon serum starvation, a significantly higher proportion of reporter cells became activated, possibly due to exposure of the TREM2 ligand phosphatidylserine on the outer leaflet of stressed cells. This activation could be blocked by inclusion of an anti-TREM2 antibody. Thus, in multiple in vitro settings of stress, TREM2-expressing cells are more able to sustain mTOR activation and suppress autophagy in a PI3K-dependent manner than are cells lacking TREM2.

### Example 4: TREM2 Deficiency Curtails Anabolic and Energetic Metabolism in BMDMs

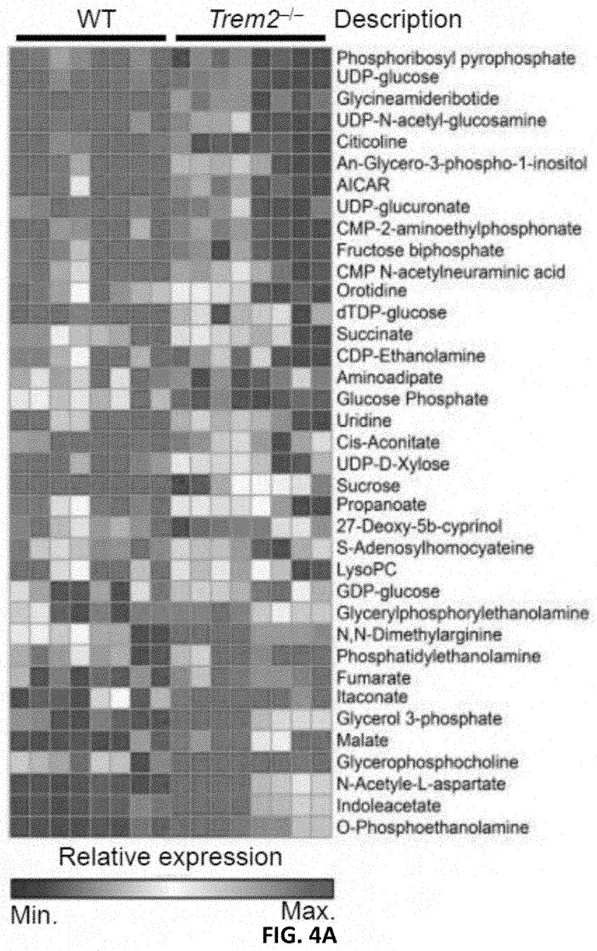

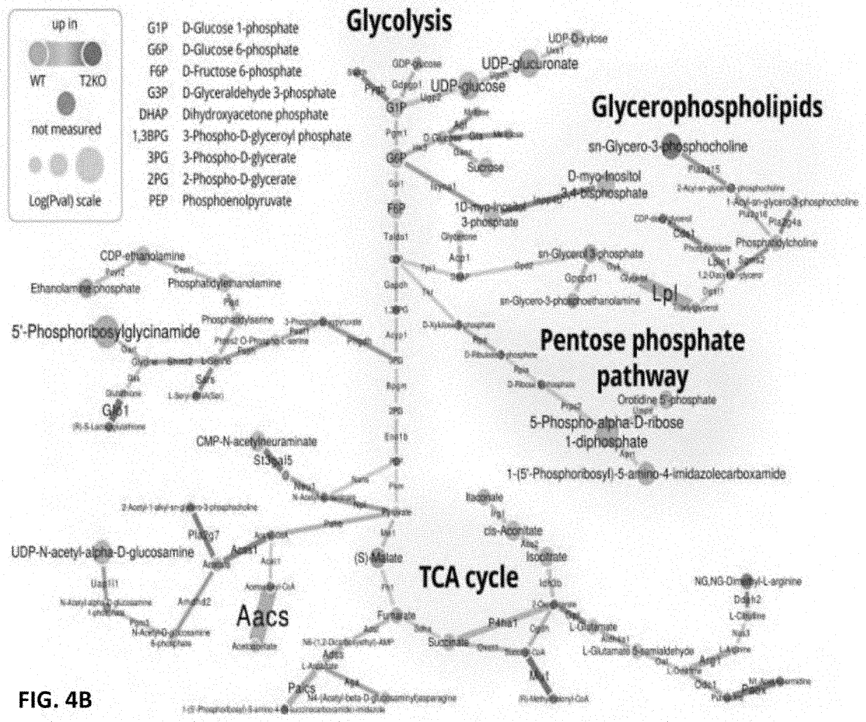

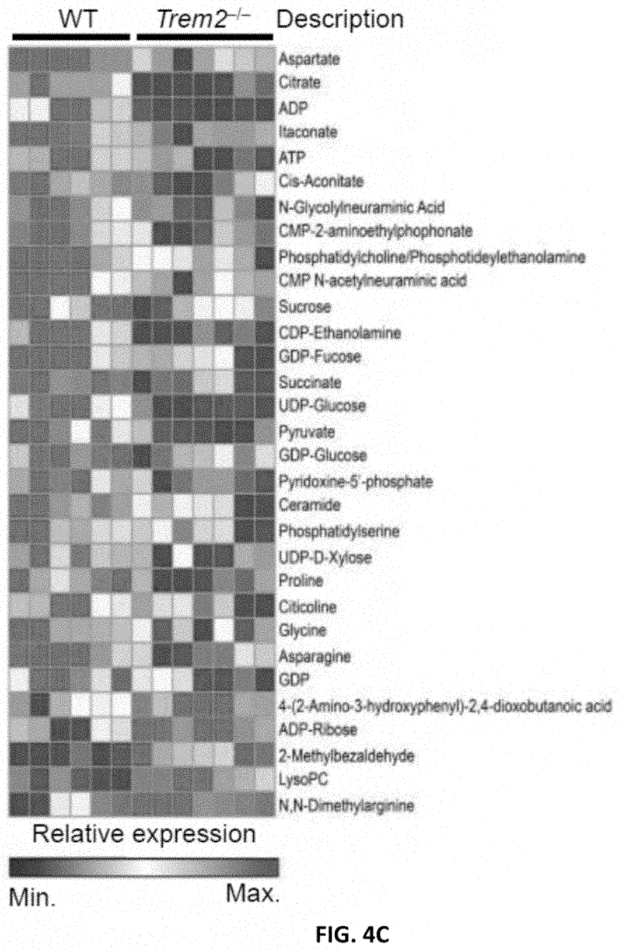

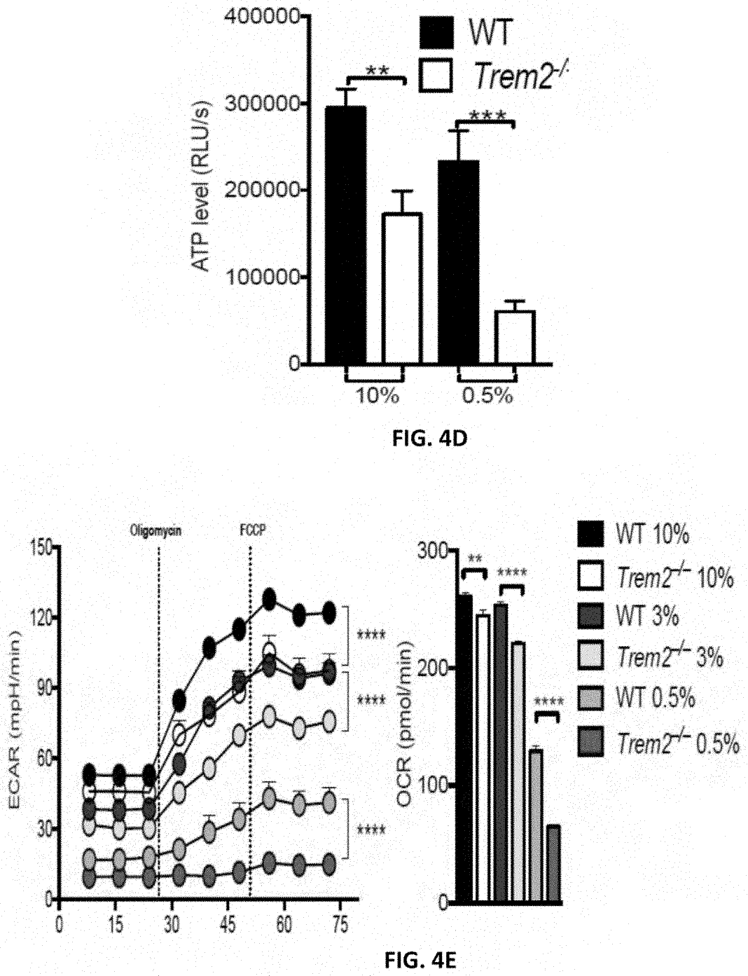

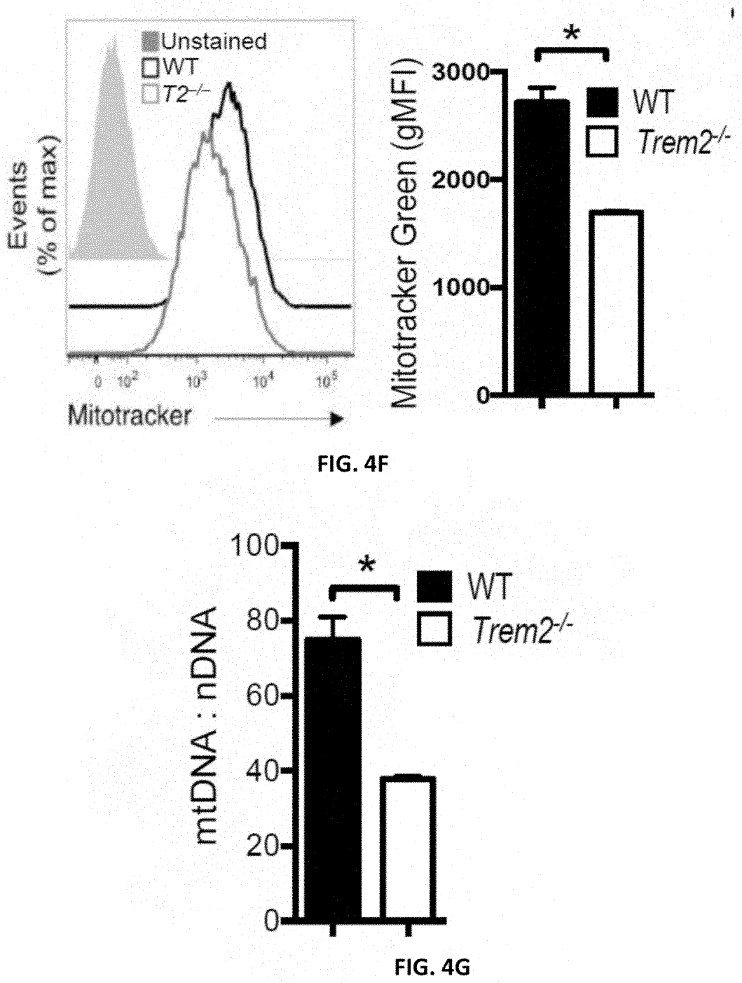

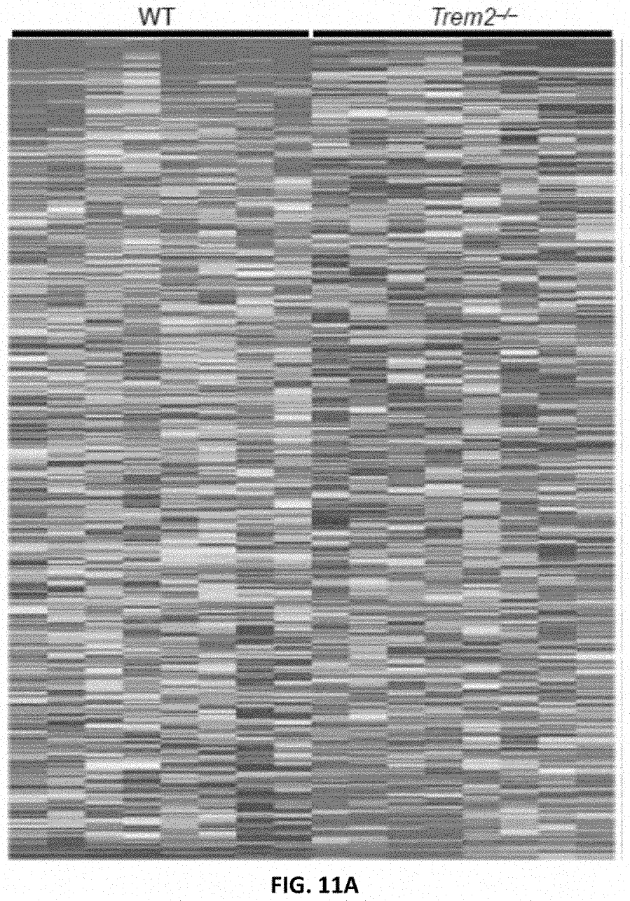

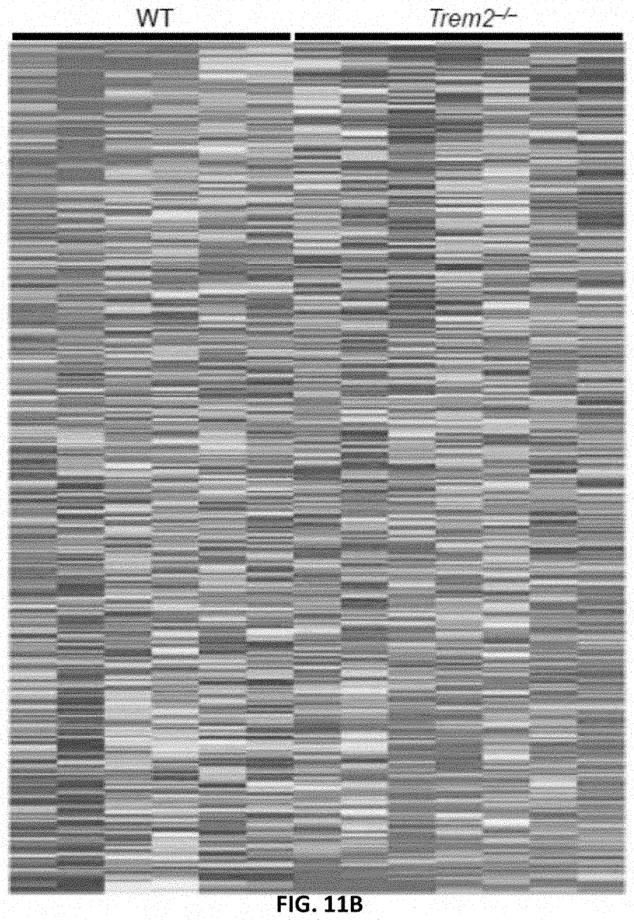

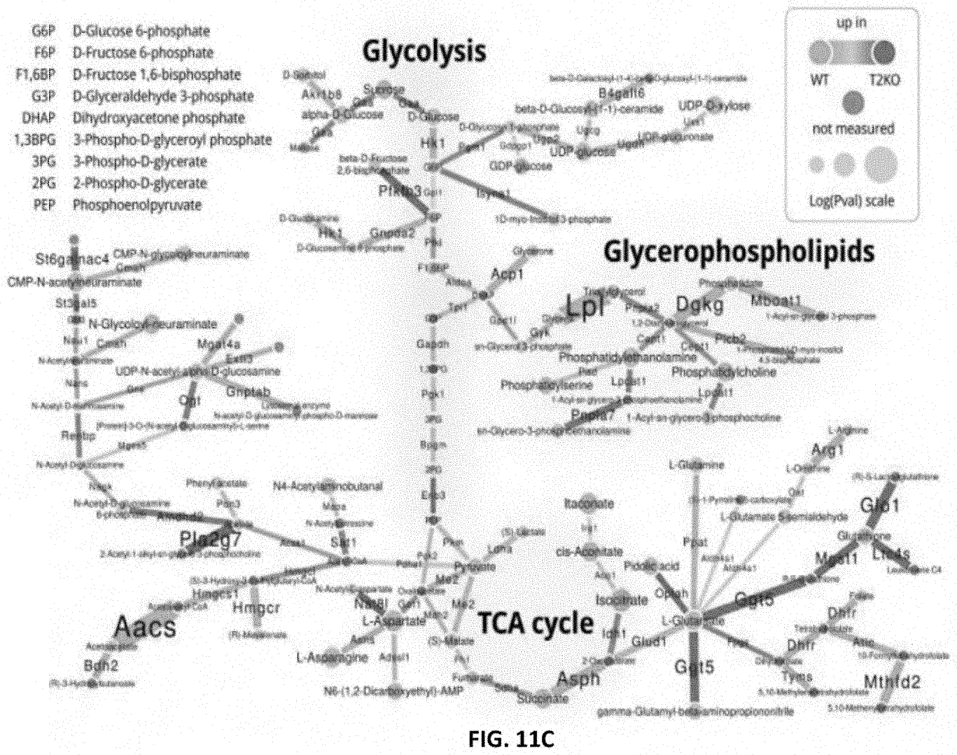

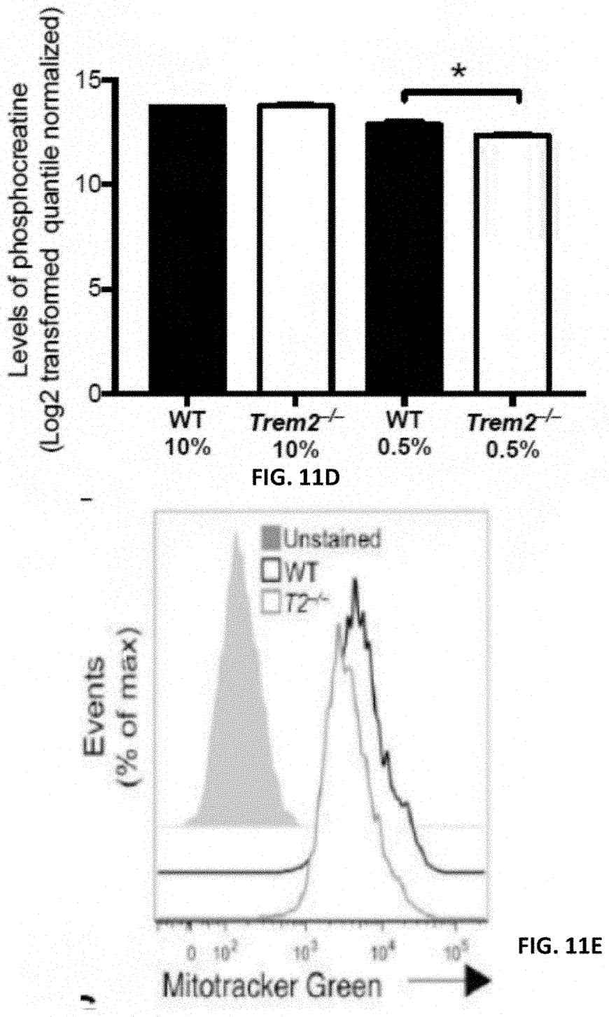

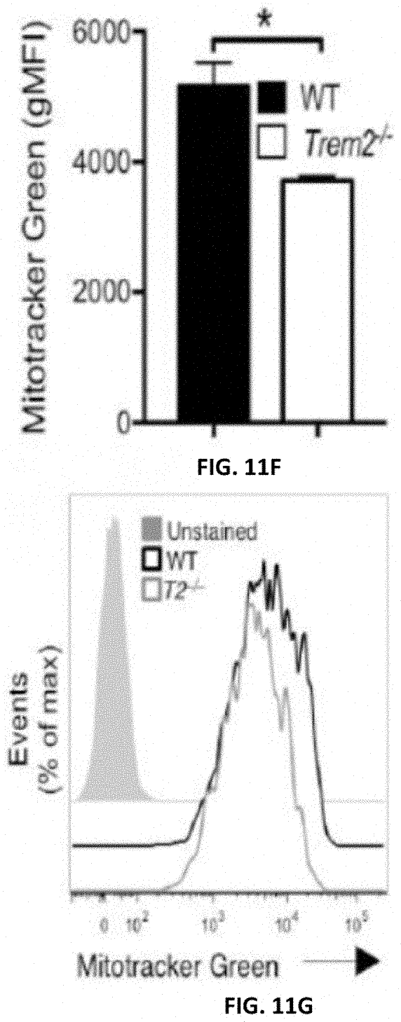

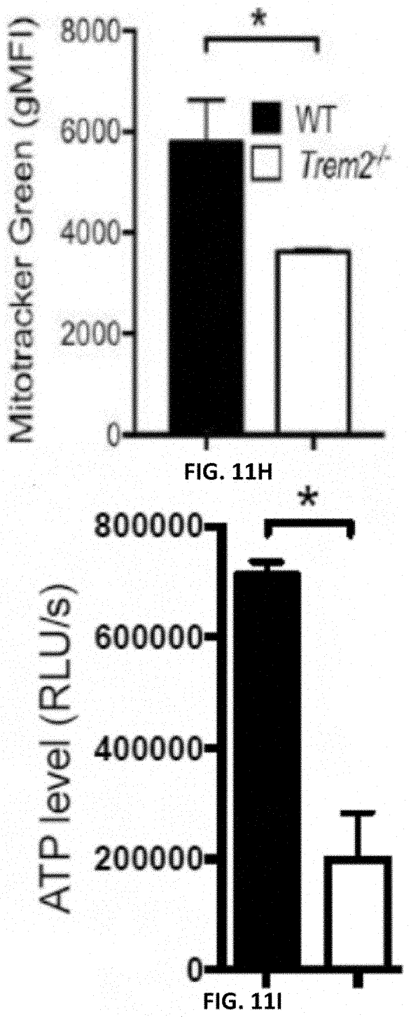

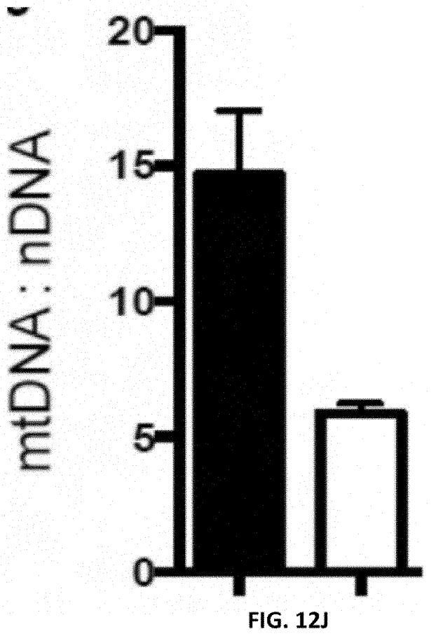

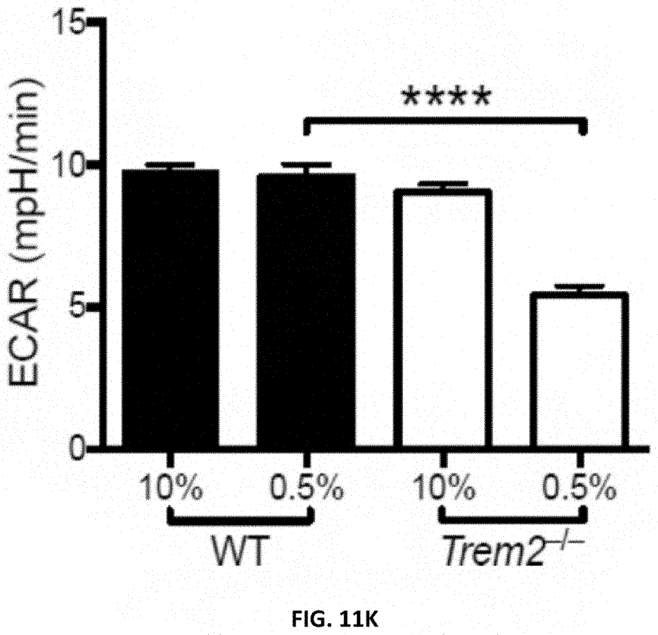

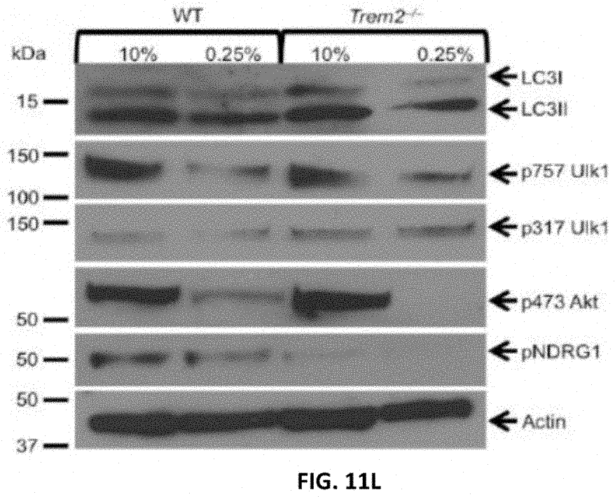

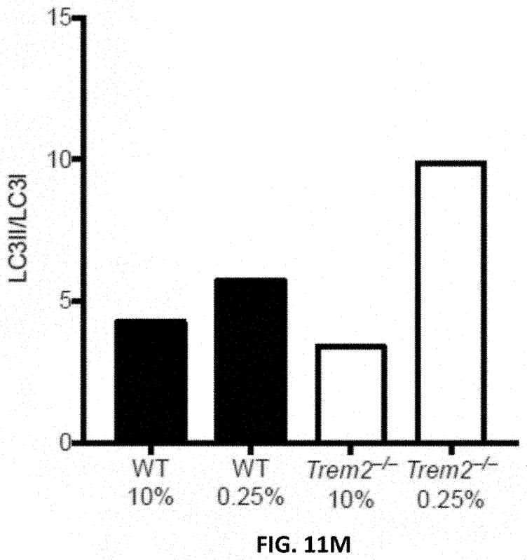

To directly demonstrate the impact of TREM2 deficiency on energetic and anabolic pathways in BMDMs, We performed mass spectrometry to quantify cellular metabolites and RNA sequencing (RNA-seq) to quantify mRNA levels of metabolic enzymes. Analysis of metabolite data alone, or in combination with RNA-Seq data by a systems-based algorithm, revealed widespread differences between WT and Trem2^{-/-} BMDMs in various metabolic pathways. Compared to WT BMDMs in optimal CSF-1, Trem2^{-/-} BMDMs cultured under the same conditions exhibited: 1) a marked decrease of key intermediates in the synthesis of nucleotides (e.g. phosphoribosyl pyrophosphate), N-glycosylated proteins (e.g. UDP-glucose), and phospholipids (e.g. CDP-ethanolamine); 2) a decrease in glycolytic metabolites (e.g. glucose 6-phosphate and fructose bisphosphate) and tricarboxylic acid (TCA) cycle intermediates (citrate and succinate); and 3) an increase in catabolic products of amino acids (e.g. indolacetate) and phospholipid precursors (e.g. glycerol 3-phosphate) (FIG. 4A, FIG. 11A). Moreover, a selective increase in malate and fumarate suggested an enhanced malate-aspartate shuttle to sustain defective NADH oxidation and NAD regeneration (FIG. 4A). Unbiased network analysis combining metabolic and RNA-seq data highlighted defects in metabolites and enzymes involved in glycolysis, TCA cycle and pentose phosphate pathway in Trem2^{-/-} BMDMs (FIG. 4B).

CSF-1 reduction further deteriorated energy and anabolic metabolism in Trem2^{-/-} BMDMs. Under these conditions, again in comparison to WT BMDMs, Trem2 BMDMs underwent a marked increase in symmetrical dimethyl arginine, indicative of protein catabolism, as well as an increase in ADP-ribose, indicative of NAD degradation (FIG. 4C, FIG. 11B-FIG. 11C). Furthermore, stores of high-energy phosphates, such as phosphocreatine and ATP, were depleted in Trem2^{-/-} BMDMs cultured in limiting CSF1 conditions (FIG. 11D). A luciferase-based ATP assay confirmed an ATP deficiency in Trem2^{-/-} BMDMs, which was exacerbated at low CSF1 concentrations (FIG. 4D). We further assessed the energy metabolism of WT and Trem2^{-/-} BMDMs using the Seahorse analyzer. A lower extracellular acidification rate (ECAR), indicative of less glycolytic flux, was noted in Trem2^{-/-} BMDMs both at baseline and after induction of maximal glycolytic capacity by oligomycin and FCCP. This deficit widened relative to WT cells as the CSF1 concentration was reduced (FIG. 4E). Trem2^{-/-} BMDMs had only a slightly reduced oxygen consumption rate (OCR) compared to WT BMDMs when cultured in standard CSF1 concentrations, indicating relatively intact oxidative phosphorylation; however, a deficit in OCR emerged as the CSF1 concentration was reduced (FIG. 4E). Trem2^{-/-} BMDMs also had fewer mitochondria than WT BMDMs on a per cell basis as measured by MitoTracker Green fluorescence and by the mitochondrial-to-nuclear DNA ratio (FIG. 4F, FIG. 4G). These findings were not restricted to BMDMs, as resting and thioglycolate-elicited peritoneal TREM2-deficient macrophages also had a lower mitochondrial mass than did WT macrophages (FIG. 11E-FIG. 11H). Cultured adult primary Trem2^{-/-} microglia recapitulated the deficiencies in energetic metabolism and mTOR signaling as well as autophagy observed in Trem2 BMDMs (FIG. 11I-FIG. 11M). Thus, lack of TREM2-mTOR signaling impairs the energy status and anabolism of BMDMs and other primary macrophages both in steady state and under energetic stress.

### Example 5: Enhanced Energy Storage or Dectin-1 Signaling can Compensate for TREM2 Deficiency In Vitro

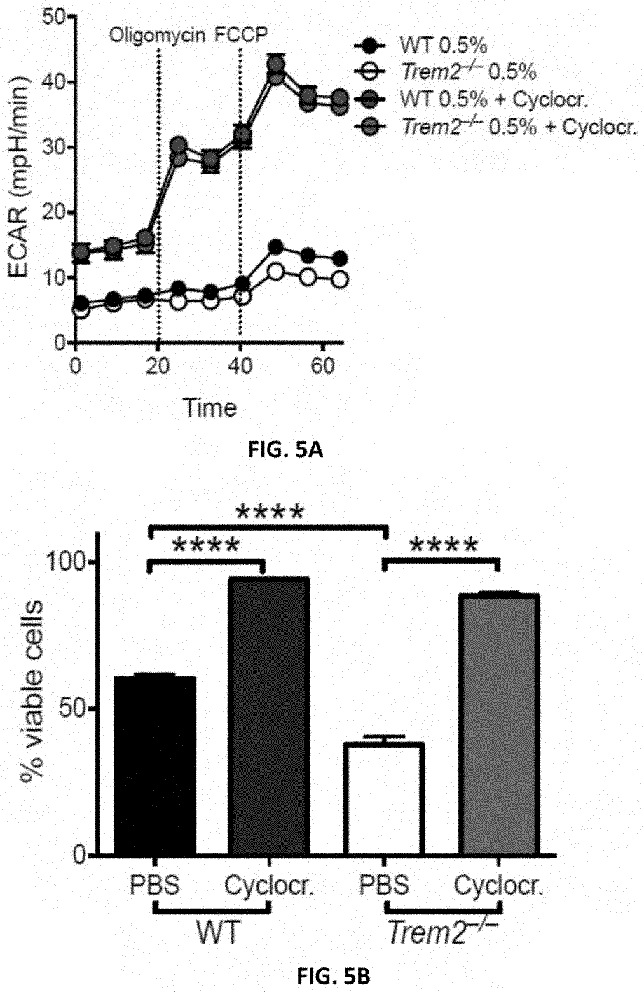

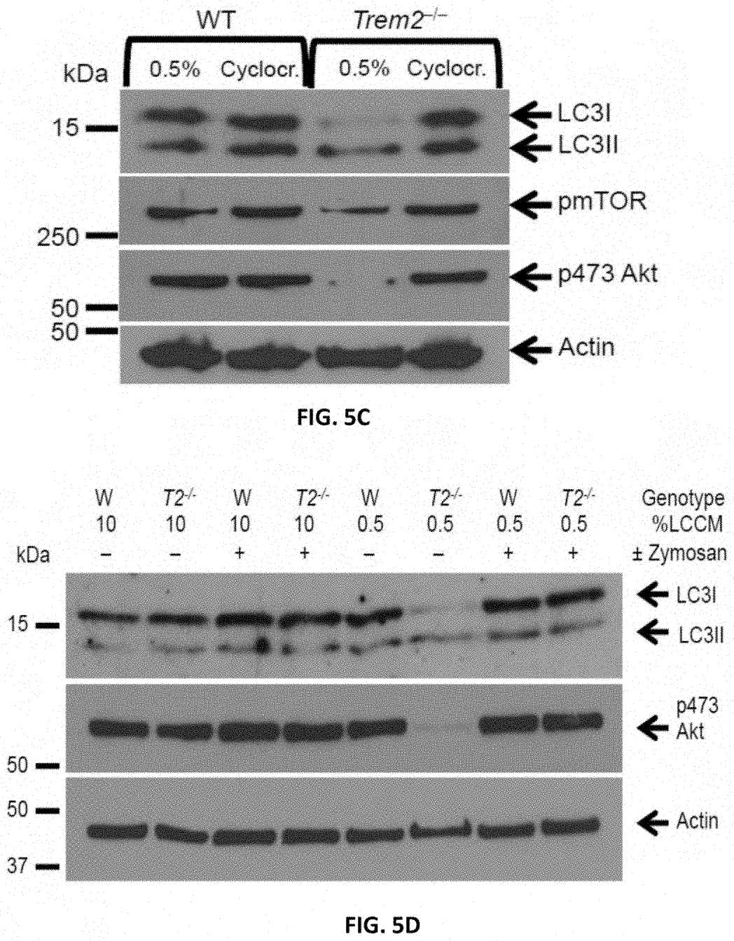

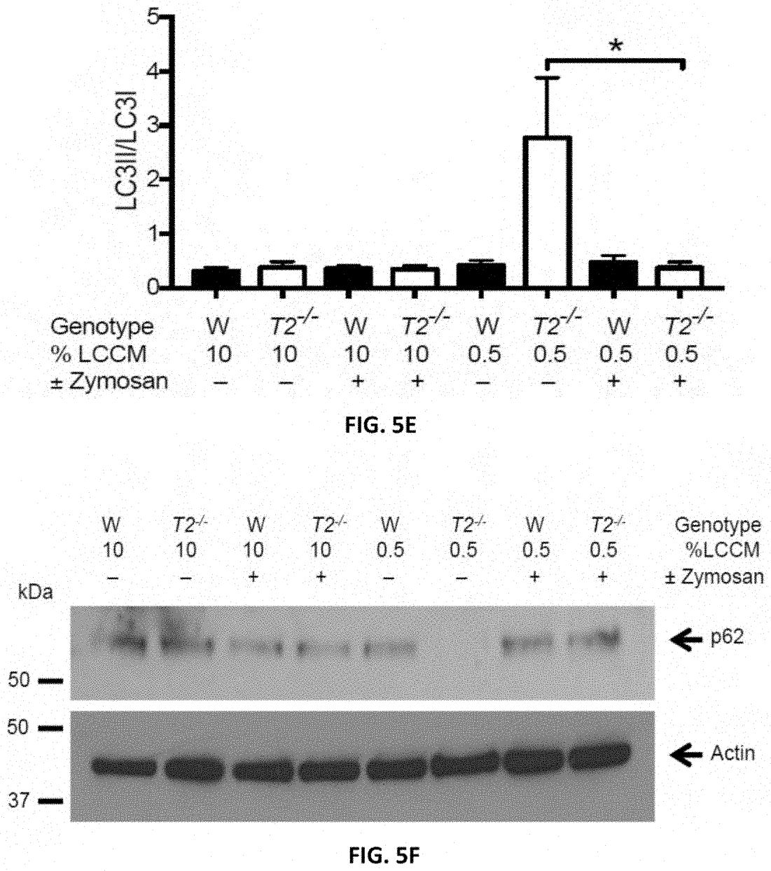

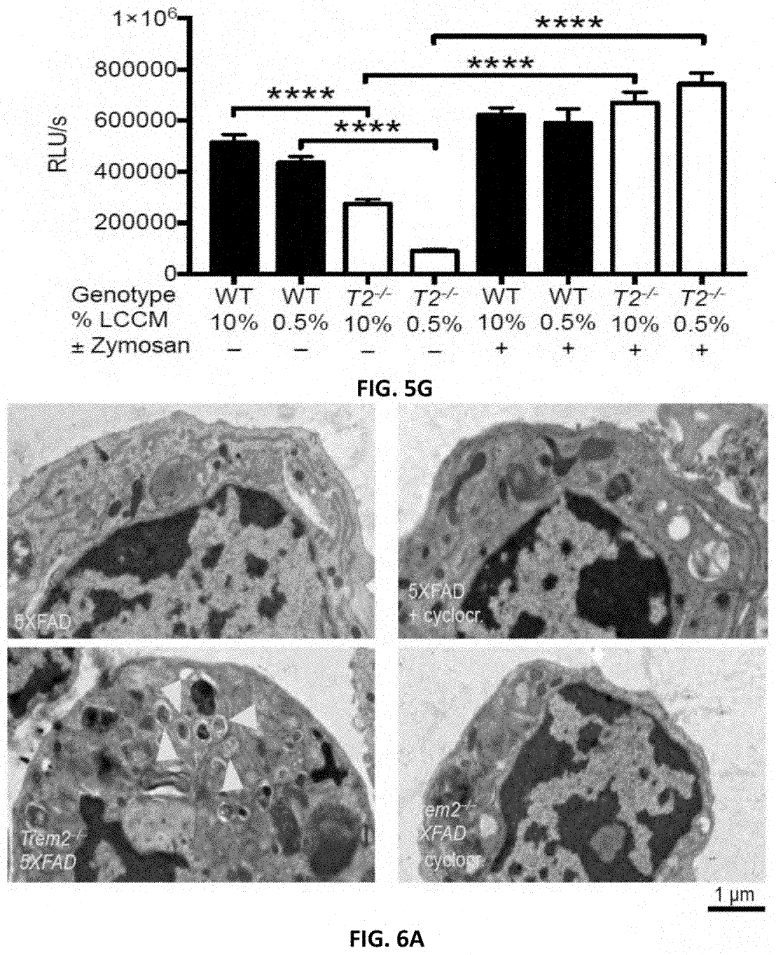

Given the dramatic effect of TREM2 deficiency on mTOR activation and energy utilization in BMDMs, We tested whether bypassing TREM2 and directly compensating for these deficits by alternative means could restore the cellular energy status of Trem2^{-/-} BMDMs. Muscle physiology studies have extensively demonstrated that creatine phosphate contributes to the regeneration of ATP and to the maintenance of uniformly high ATP/ADP ratios in muscle fibers (Walker, 1979). Moreover, the creatine analog 1-carboxymethyl-2-iminoimidazolidine (cyclocreatine) can, upon phosphorylation, generate a long-acting phosphagen that can effectively sustain cellular ATP levels during increased energy demand. Thus, We tested whether addition of cyclocreatine to the culture medium could rescue energetic metabolism in Trem2-deficient BMDMs. Indeed, incubation with cyclocreatine improved ECAR, which was accompanied by less autophagy, increased mTOR signaling and viability (FIG. 5A-FIG. 5C and data not shown).

To test whether engagement of receptors that elicit signaling pathways similar to those of TREM2 could also mitigate autophagy and support cell survival, Trem2^{-/-} and WT BMDMs were cultured with depleted zymosan, a selective ligand of dectin-1, which activates Syk and PI3K signaling independent of DAP12 (Dambuza and Brown, 2015). Dectin-1 activation curbed autophagy in CSF1-starved TREM2-deficient BMDMs to levels seen in WT BMDMs in low CSF-1, as indicated by a reduction in the LC3II/LC3I ratio (FIG. 5D, FIG. 5E) along with increased amounts of p62 (FIG. 5F). Treatment with zymosan also enhanced cellular ATP levels in Trem2^{-/-} BMDMs, restoring them to WT BMDM levels. (FIG. 5G). Thus, alternative energetic and signaling pathways can compensate for lack of TREM2 signaling.

### Example 6: Cyclocreatine Rescues Microgliosis and Clustering and Moderates Neurite Dystrophy In Vivo

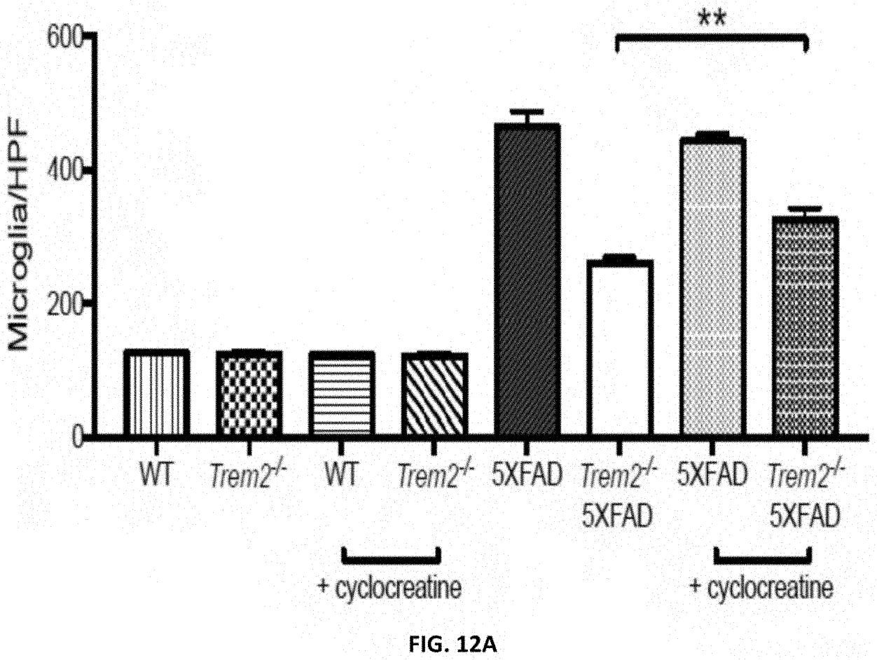

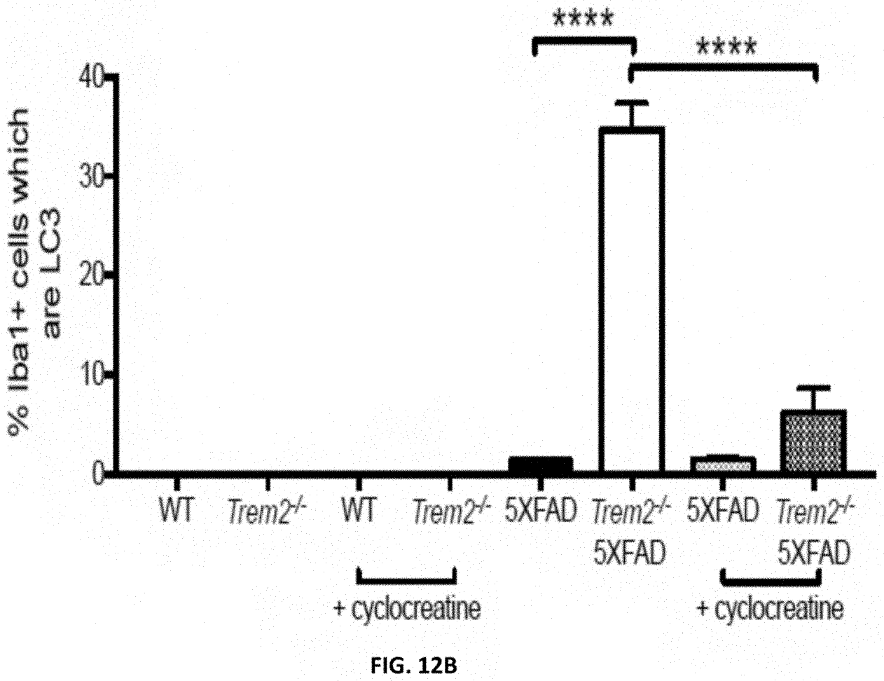

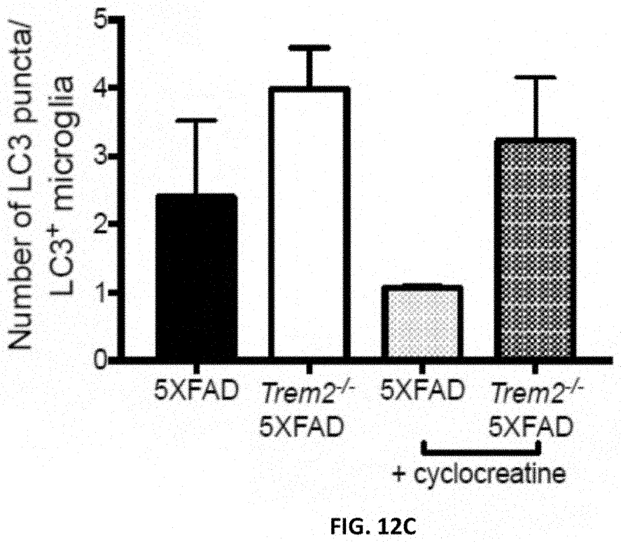

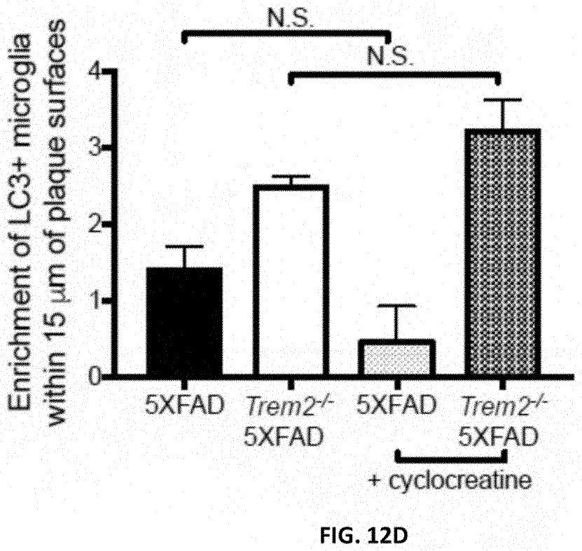

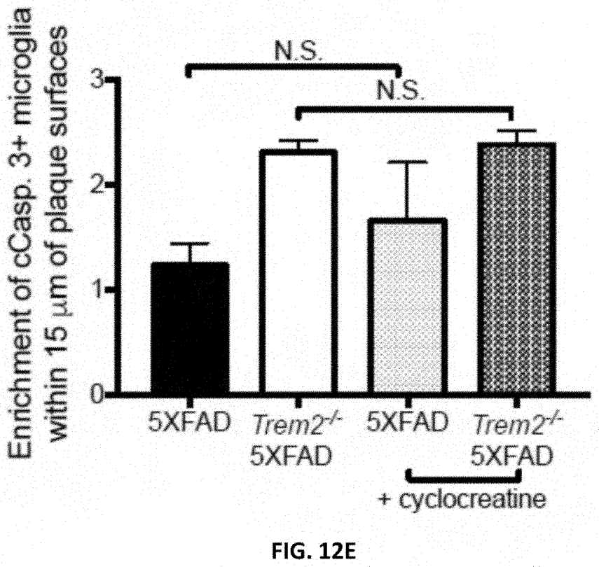

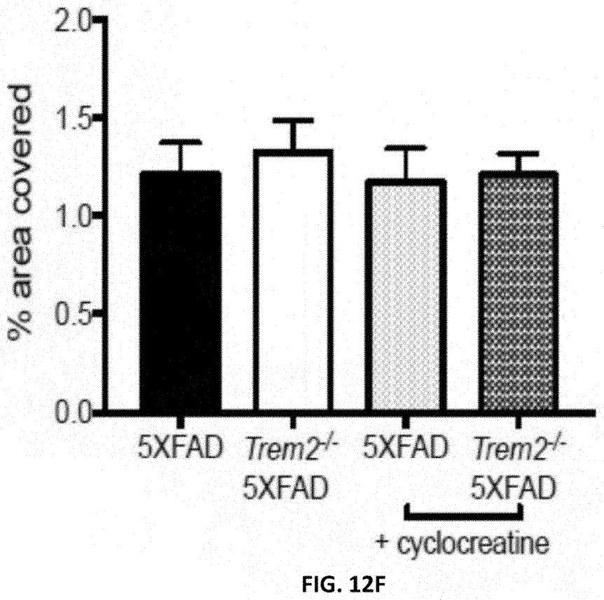

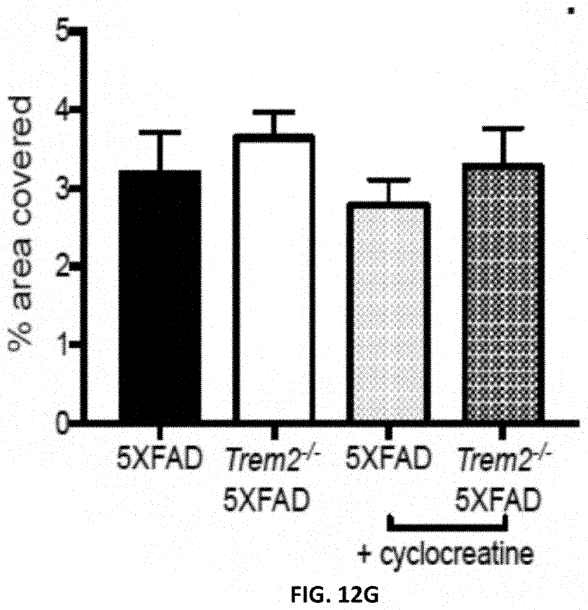

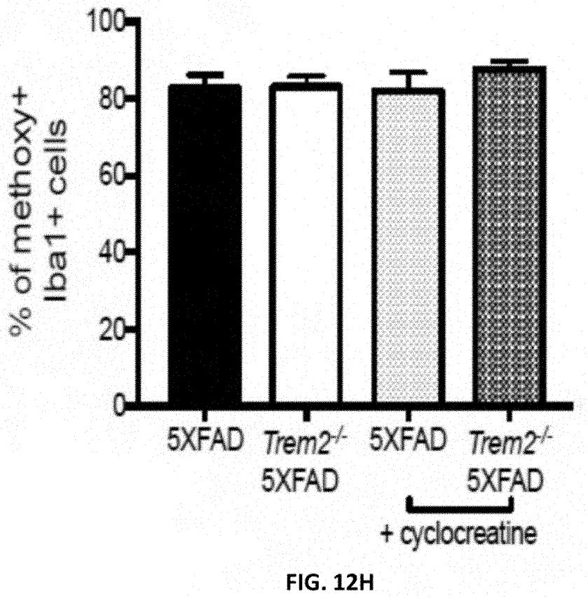

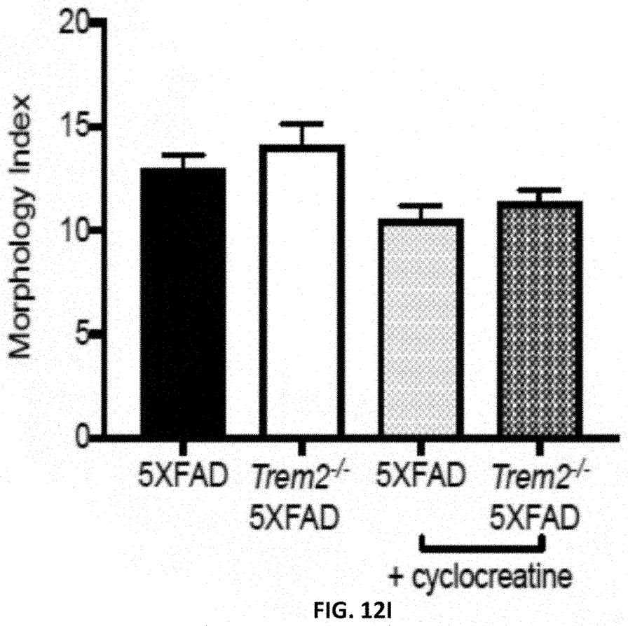

Because cyclocreatine rescued metabolism and viability and suppressed autophagy in Trem2^{-/-} BMDMs in vitro and given previous studies showing that cyclocreatine is passively transported across membranes and can accumulate and function as a phosphagen in the mouse brain in vivo, We asked whether dietary supplementation with cyclocreatine could rescue microglial function and suppress autophagy in vivo in Trem2^{-/-} 5XFAD mice. The drinking water of 5XFAD and Trem2^{-/-} 5XFAD mice was supplemented with cyclocreatine from 10 weeks of age until 8 months of age. Remarkably, significantly fewer multivesicular/multilamellar structures were seen by TEM in microglia in Trem2^{-/-} 5XFAD mice treated with cyclocreatine than in microglia in untreated mice (FIG. 6A, FIG. 6B). Corroborating this with confocal microscopy, the percentage of LC3⁺ microglia, the number of LC3 puncta/cell, and the percentage of cleaved caspase-3⁺ microglia were all significantly decreased in Trem2^{-/-} 5XFAD mice treated with cyclocreatine (FIG. 12B and FIG. 6C, FIG. 6E, FIG. 6F,). Furthermore, the number of microglia/high powered field (HPF) in plaque-bearing regions of the cortex and clustering of microglia around plaques were both significantly increased (FIG. 6C-FIG. 6D and FIG. 12A) in Trem2^{-/-} 5XFAD mice treated with cyclocreatine compared to untreated Trem2^{-/-} 5XFAD mice. These findings indicate that dietary supplementation with cyclocreatine is sufficient to partially rescue the defect in microgliosis and microglial clustering around plaques in Trem2^{-/-} 5XFAD mice, while concomitantly mitigating autophagy and death of the microglia.

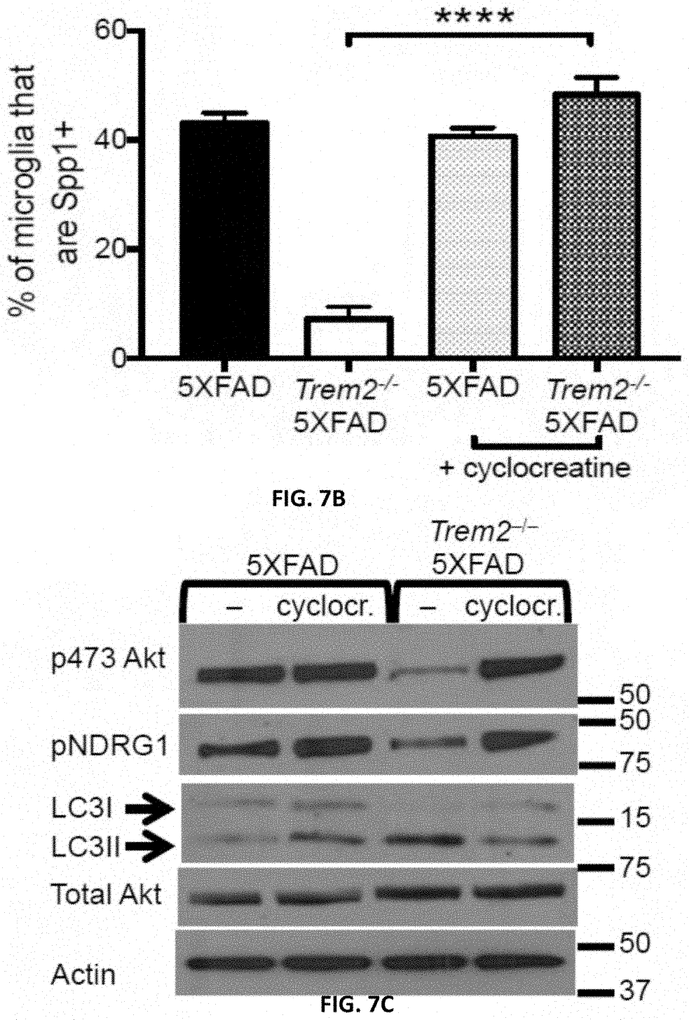

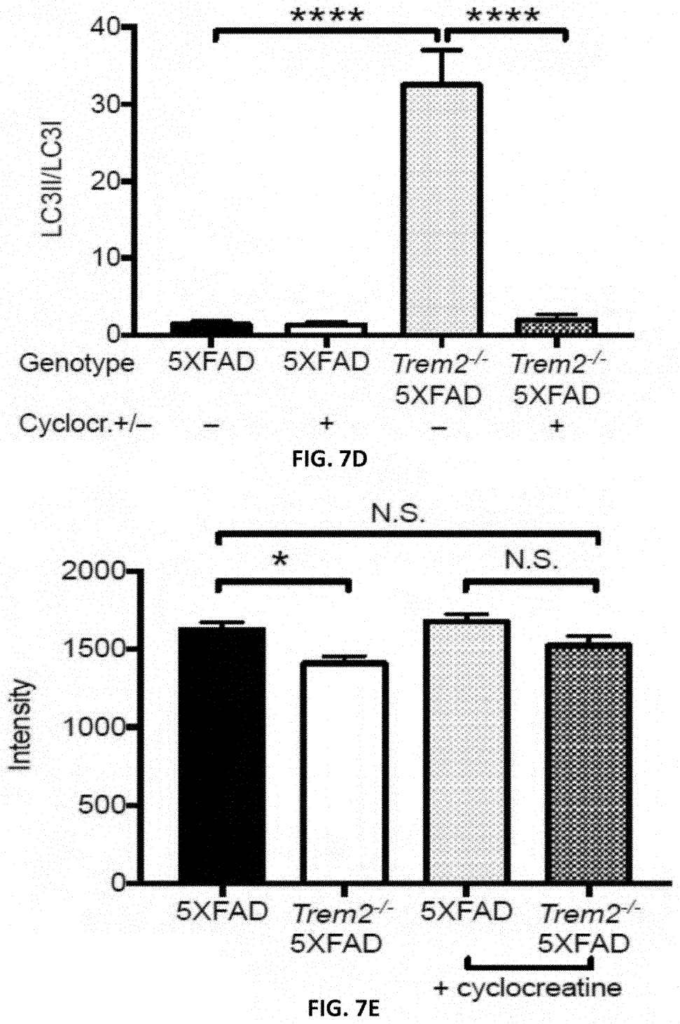

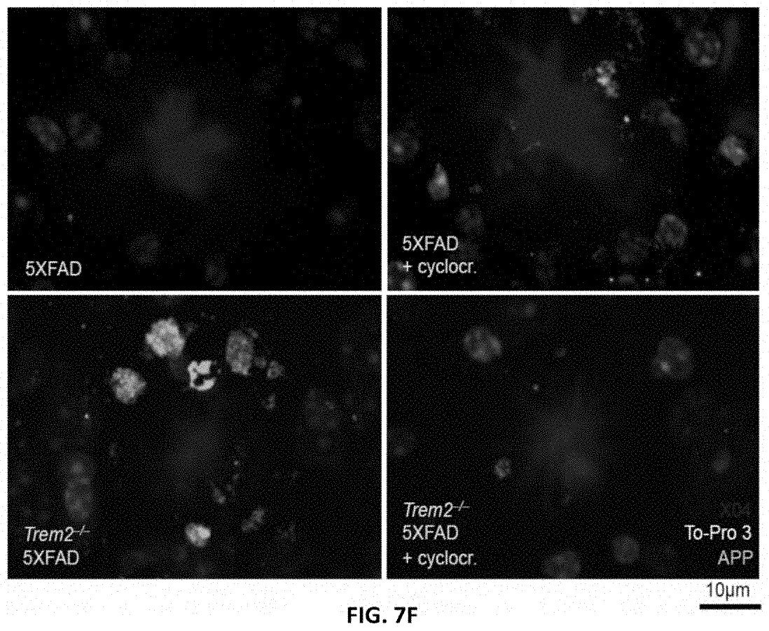

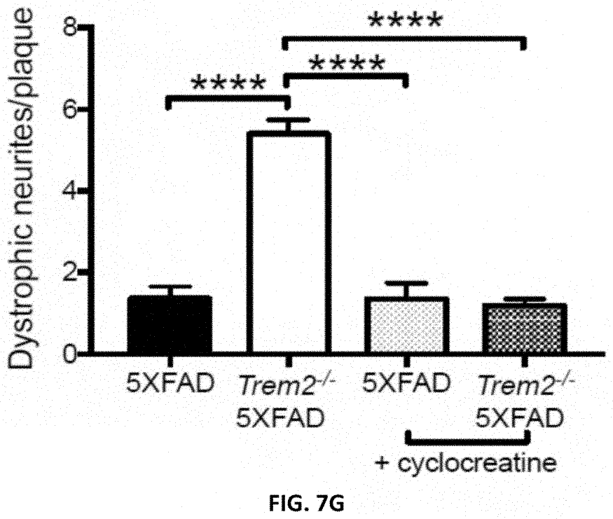

To assess the impact of cyclocreatine on microglial activation, which is also impaired in Trem2^{-/-} 5XFAD mice, We quantified the percentage of microglia that expressed the microglial activation marker osteopontin (Spp1) in 5XFAD and Trem2^{-/-} 5XFAD mice, a protein that, in the brain, is specifically upregulated in microglia in the context of Aβ deposition. Untreated Trem2^{-/-} 5XFAD mice had very few Spp1⁺ microglia, while 5XFAD, cyclocreatine-treated 5XFAD, and cyclocreatine-treated Trem2^{-/-} 5XFAD mice all had significantly more Spp1⁺ microglia (FIG. 7A, FIG. 7B). Moreover, biochemical analysis of microglia isolated ex vivo demonstrated that cyclocreatine treatment of Trem2^{-/-} 5XFAD mice also restored microglial mTOR signaling and significantly limited autophagy compared to untreated Trem2^{-/-} 5XFAD mice (FIG. 7C, FIG. 7D).

As a major function of TREM2 in vivo is enabling microglia to form a barrier around plaques that prevents spreading of Aβ fibrils and alleviates dystrophy of plaque-adjacent neurites. We asked whether cyclocreatine treatment of Trem2^{-/-} 5XFAD mice impacted plaque morphology and/or neuronal dystrophy. While plaques in untreated Trem2^{-/-} 5XFAD mice had a lower density than those in 5XFAD mice as measured by methoxy-X04 staining intensity, the density of plaques in cyclocreatine treated Trem2^{-/-} 5XFAD mice resembled that of plaques in 5XFAD mice (FIG. 7E), although plaque shape complexity was not significantly altered (FIG. 12K). Despite reducing plaque density, cyclocreatine did not moderate plaque accumulation or the engulfment of plaque particulates by microglia, at least at this time point (FIG. 12F-FIG. 12K). As APP is known to accumulate in dystrophic neurites, We used APP deposition in distinct rounded particles around plaques to assess neurite dystrophy. Cyclocreatine treatment of Trem2^{-/-} 5XFAD mice significantly reduced plaque-associated neurite dystrophy compared to untreated Trem2^{-/-} 5XFAD mice to levels observed in 5XFAD mice (FIG. 7F, FIG. 7G). Taken together, these data indicate that cyclocreatine administration improves microglial metabolism and the protective response to Aβ plaques in TREM2-deficient 5XFAD mice.

Discussion

Increasing evidence supports the hypothesis that the microglial response to AD lesions controls disease progression. Toll-like receptors and NOD-like receptors have been previously implicated in the microglia response to Aβ accumulation and shown to mediate an inflammatory response that contributes to pathology. To sustain cytokine secretion, these receptors induce a striking metabolic reprogramming, which consists of a switch from fatty acid metabolism and oxidative phosphorylation to glycolysis (O'Neill and Pearce, 2016). In our study, TREM2 emerges as an innate immune receptor that impacts microglia metabolism in AD through a distinct mechanism, which consists of basic activation of mTOR signaling that supports long-term cell trophism, survival, growth, and proliferation, rather than drastic metabolic reprogramming. This function of TREM2 is reminiscent of the tonic function of the B cell antigen receptor in mature B cells, which delivers survival signals through PI3-K. Likewise, cell membrane phospholipids and lipoprotein particles may continuously engage TREM2, inducing tonic mTOR signaling through upstream activators, such as PI3-K, PDK1 and AKT, which are recruited by the TREM2-associated signaling subunits DAP12 and DAP10. This concept provides a unifying mechanism to explain the reported broad and long-term impact of TREM2 on diverse microglial functions, such as survival, proliferation, clustering around plaques, as well as phagocytosis of apoptotic cells and myelin debris.

We found that the defective mTOR signaling in TREM2-deficient microglia is associated with a compensatory increase of autophagy in vitro and in vivo in AD. Reduced glycolysis and autophagy are known to attenuate inflammation and, indeed, microglia from 5XFAD mice lacking TREM2 weakly express inflammatory mediators in comparison to microglia from 5XFAD mice. Moreover, autophagy may also enhance microglial clearance of Aβ, as it does in neurons. However, a long-term defect in mTOR activation results in global microglial dysfunction, reduced cell viability and proliferation, as demonstrated by increased caspase-3 activation in microglia and by the previously reported increase in dying microglia around plaques in Trem2^{-/-} 5XFAD mice. Thus, while increased autophagy may be beneficial in reducing inflammation and Aβ load in the short-term, a defect in mTOR signaling is detrimental and severely impairs microglia fitness and capacity to respond to Aβ accumulation in the long-term.

TREM2-deficient microglia have long been thought to improperly remain in a homeostatic state during neurodegenerative disease rather than responding appropriately to pathology, a paradigm that has been supported by transcriptomic analysis of these cells. However, for the first time, We demonstrate that on a biochemical and ultrastructural level, TREM2-deficient microglia adopt a severely divergent cellular state that does not reflect homeostasis during neurodegeneration, with a dramatic loss of mTOR signaling and robust induction of autophagy. These results suggest that TREM2-deficient microglia are not simply ignoring plaque pathology, but rather that they are being actively driven into a stressed state that is normally compensated by TREM2-dependent survival signals. An important implication of this finding is that microglia in a neurodegenerative environment probably receive not only positive activating signals but also negative cytotoxic signals. Thus, it may not be microglial activation per se that is required to protect against neurodegeneration, but rather avoidance of a dysfunctional, low-energy state induced by the disease. Based on our findings, previous reports of impaired microglial activation in a variety of settings may be due to either impaired recognition of activating signals or to impaired resistance to cytotoxic signals—two possibilities that can be distinguished by the strength of mTOR signaling. Counteracting such dysfunction by metabolic compensation may also represent a fundamentally distinct therapeutic approach.

Along these lines, our study shows that the defect in mTOR-mediated metabolic activation in TREM2-deficient cells can be corrected in vitro through the creatine kinase pathway or by triggering the dectin-1 pathway, which transmits intracellular signals similar to those of TREM2. Based on these results, We adopted a therapeutic strategy based on the use of cyclocreatine, an analog of creatine that crosses membranes, enters the brain (Woznicki and Walker, 1979), can be phosphorylated and dephosphorylated by creatine kinases, and can generate a supply of ATP. Remarkably, We found that administration of dietary cyclocreatine throughout the progression of Aβ accumulation improves microglia viability, numbers and clustering around Aβ plaques. As a result, plaques are denser and, most importantly, plaque-associated neurite dystrophy is greatly reduced. Although cyclocreatine treatment was not sufficient to reduce the overall Aβ plaque accumulation, this may depend on time point chosen for analysis and/or cyclocreatine dosage and duration of treatment. While the creatine kinase pathway has been previously recognized to play an important role in the CNS in neurotransmitter release, membrane potential maintenance, Ca²⁺homeostasis, and ion gradient restoration, our results indicate that this system may also be exploited for sustaining microglial metabolism. It is noted that in certain settings cyclocreatine inhibits creatine kinase and/or has systemic effects such as alteration in pancreatic hormones and glucose metabolism. As this is the case and creatine and creatine analogs like cyclocreatine are available over the counter for use we must emphasize that these findings are the result of proof of principal studies and We do not advise the use cyclocreatine as a preventative treatment for AD. Future studies will be required to precisely define the mechanisms through which cyclocreatine impacts microglial responses to Aβ. Additionally, it will be important to determine whether cyclocreatine has any impact on proteolytic shedding of TREM2 from microglia, which results in the release of soluble TREM2 with potential pro-survival functions. Altogether, our study provides proof of principle that strategies aimed at sustaining microglial metabolism may be promising for therapeutic intervention in AD and other neurodegenerative diseases linked to TREM2 deficiency and microglial dysfunction in general.

Experimental Model and Subject Details

Mice.

Mice were of mixed sexes. Mice within experiments were age and sex matched. For studies using 5XFAD and Trem2^{-/-} 5XFAD animals all animals were 8 months of age at the time of use. For bone marrow and primary microglia mice were used from 6 weeks of age until 12 weeks of age. Mice used in this study include WT C57BL/6J, 5XFAD, Trem2^{-/-}, and Trem2^{-/-} 5XFAD animals. All animals were backcrossed until at least >98% C57BL/6J confirmed by genotype wide microsatellite typing. Mice were housed under specific pathogen free conditions. Mice from different genotypes were cohoused. Mice did not undergo any procedures prior to their stated use. For cyclocreatine treatment mixed litters of sex matched mice were randomly assigned to experimental groups. All studies performed on mice were done in accordance with the Institutional Animal Care and Use Committee at Washington University in St. Louis approved all protocols used in this study.

Human Post-Mortem Samples.

Characteristics of donors of human post-mortem brain tissue at the time of collection is indicated in Table 1. Samples from 7 R47H, 4 R62H, and 8 case matched AD patients were examined. Samples were obtained from the Knight Alzheimer's Disease Research Center at Washington University. Protocol numbers: Healthy Aging and Senile Dementia (HASD) Morphology Core: 89-0555 and Program Project: Alzheimer's Disease Research Center (ADRC): 89-0556.

Cell Lines and Primary Cells.

Bone marrow derived macrophages and microglia were prepared from sex and age matched mice. To prepare bone marrow-derived macrophages, femurs and tibias were removed and flushed with PBS. Cells were counted and plated at 2.5×10⁶ cells/100 mm petri dish in RPMI supplemented with Glutamax, penicillin/streptomycin, non-essential amino acids, pyruvate, and 10% heat inactivated fetal bovine serum (complete RPMI) and 10% L-cell conditioned medium (LCCM). Cells were cultured for 4-5 days before use. Microglia were prepared as previously described. Briefly, brains were dissociated by using a Neural Tissue Dissociation Kit (T) (Miltenyi Biotech Cat. Number 130-093-231). Cells suspensions were labeled with anti-mouse CD45 magnetic beads and isolated on LS columns (Miltenyi Biotic). Cells were plated onto poly-L-lysine coated polystyrene plates in complete RPMI supplemented with 20% LCCM and 10 ng/ml human TGF-β. Media was changed on day 3 post plating and cells were used 5-7 days post plating.

Trem2 reporter cells were maintained in 10% FBS in RPMI-1640 supplemented with sodium pyruvate, GlutaMAX, and penicillin/streptomycin. Trem2 reporter cells were based on the 2B4 NFAT-GFP cells developed by Arase et al. The sex of the mouse from which 2B4 t-cell hybridoma cells were derived has not been reported.

Method Details

Mice.

The generation of Trem2^{-/-} and Trem2^{-/-} 5XFAD mice has been described previously. All mice were on a C57BL/6 background. Age and sex matched mice were used for all experiments; experimental cohorts of mice were cohoused from birth to control for the microbiota.. For in vivo cyclocreatine treatment 10-week old mice were put on cyclocreatine-containing water, treatment was continued until mice reached 8 months of age (Santa Cruz SC-217964 S). Desired intake of cyclocreatine was approximately 0.28 mg/g of body weight/day, which is approximately the same as the standard creatine dose used in humans of 285 mg/kg of body weight/day. Cyclocreatine was administered in drinking water at a final concentration of 2.33 mg/ml. The Institutional Animal Care and Use Committee at Washington University in St. Louis approved all protocols used in this study.

Human Post-Mortem Brain Tissue.

Paraffin-embedded sections (8 um) from the frontal cortex of individuals carrying the common variant (CV) of TREM2 (8) or heterozygous for the CV and either R47H (7), R62H (4) were obtained from the Knight Alzheimer's Disease Research Center at Washington University. Protocol numbers: Healthy Aging and Senile Dementia (HASD) Morphology Core: 89-0555 and Program Project: Alzheimer's Disease Research Center (ADRC): 89-0556. R47H and R62H carriers were case matched for age, gender, and CERAD-Reagan plaque score to CV TREM2 control individuals. Detailed demographic characteristics are provided in Table 1.

Immunohistochemistry of Human Post-Mortem Brain Tissue.

Brain sections were deparaffinized with xylene and rehydrated with decreasing concentrations of ethanol. Antigen retrieval was performed by incubating sections for 20 minutes in a 95° C. citrate buffer bath (10 mM sodium citrate, 0.05% Tween-20, pH 6.0) prior to staining. Sections were blocked in 3% goat serum in PBS for 30 minutes at room temperature (RT) followed by incubation with rabbit anti-Iba1 (1:250, Wako; catalog no. 019-19741) overnight at 4° C. Sections were washed in PBS and incubated for 1 hour at room temperature (RT) with methoxy-X04 (20 μg/ml) (Tocris Bioscience #4920) and anti-rabbit DyLight 549 (Vector Laboratories DI-1549). Sections were washed and incubated overnight in anti-LC3A/B Alexa Fluor 488 (Cell Signaling Technologies #13082). Sections were washed and mounted using Fluoromount G (SouthernBiotech #0100-01) and images were collected using a Nikon A1Rsi+ confocal microscope. Images were then processed with Imaris 7.7 (Bitplane).

Cell Culture and Biochemical Assays.

To prepare bone marrow-derived macrophages, femurs and tibias were removed and flushed with PBS. Cells were counted and plated at 2.5×10⁶ cells/100 mm petri dish in RPMI supplemented with Glutamax, penicillin/streptomycin, non-essential amino acids, pyruvate, and 10% heat inactivated fetal bovine serum (complete RPMI) and 10% L-cell conditioned medium (LCCM). Cells were cultured for 4-5 days before use. Microglia were prepared as previously described. Briefly, brains were dissociated by using a Neural Tissue Dissociation Kit (T) (Miltenyi Biotech Cat. Number 130-093-231). Cell suspensions were labeled with anti-mouse CD45 magnetic beads and isolated on LS columns (Miltenyi Biotic). Cells were plated onto poly-L-lysine coated polystyrene plates in complete RPMI supplemented with 20% LCCM and 10 ng/ml human TGF-β. Media was changed on day 3 post plating and cells were used 5-7 days post plating. ATP concentrations were determined with an ATP Determination Kit (Invitrogen).

Microglia Sorting.

Microglia were isolated from the indicated animals as previously described. CD45⁺, CD11b⁺, F4/80⁺ (Biolegend Cat. Number 103134, eBioscience Cat. Numbers 11-0112 and 17-4801) cells in the brain were fluorescence-activated cell-sorted (FACS) directly into RLT-plus lysis buffer for microarray or 2% FBS in PBS for TEM or immunoblot lysates. For microarray RNA extraction was performed using a RNeasy micro kit (QIAGEN). Microarray hybridization (Affymetrix MoGene 1.0 ST array) and data processing were performed at the Washington University Genome Center. For normalization, raw data was processed by Robust Multi-Array (RMA) method and genes were pre-filtered for expression value >120 expression units, a cut-off above which genes have a 95% chance of expression demonstrated in Immgen data set, which uses the same array platform. QIAGEN IPA analysis was performed by comparing fold change and p-values for all genes 5XFAD and Trem2-deficient 5XFAD microglia. Heatmaps and hierarchical clustering were generated from preselected gene-lists using Morpheus. Microarray data has been deposited at GEO:GSE65067.

Immunoblotting.

BMDM or microglia were lysed in RIPA buffer (50 mM Tris, 150 mM NaCl, 1% SDS, and 1% Triton X100) containing PMSF, leupeptin, activated sodium orthovanidate, apoprotinin, and phosphatase inhibitor cocktail 3 (Sigma Aldrich Cat. Number P0044). Lysates were flash frozen on dry ice and stored at −80° C. until use. Lysates were thawed and 4×LDS running buffer and 10% β-mercaptoethanol were added. Lysates were heated to 95° C. for 10 minutes and run on either a 15% polyacrylamide gel with a 4% stacking gel, a 12% bis-tris gel (Nupage), or a 4-12% bis-tris gel (Nupage). Proteins were transferred to nitrocellulose and blocked for 1 hour at RT in 5% milk in Tris buffered saline with 0.05% Tween 20 (TBST). Membranes were incubated in primary antibody overnight at 4° C. Membranes were subsequently washed and incubated in Lienco anti-rabbit HRP for 1 hour at RT, washed, and developed using either SuperSignal West Pico Chemiluminescent Substrate or a combination of SuperSignal West Pico Chemiluminescent Substrate and SuperSignal West Femto Chemiluminescent Substrate.

Metabolite Profiling by EIS-MS/MS.

BMDMs were cultured in either 0.5% or 10% LCCM overnight in complete RPMI. Polar metabolites were extracted according to General Metabolics protocol for extraction of polar metabolites from adherent mammalian cell culture. Briefly, cells were washed in pre-warmed 75 mM ammonium carbonate in water. Metabolites were extracted by addition of 70° C. 70% ethanol for 3 minutes. Ethanol was removed and plates were washed with additional 70° C. 70% ethanol. Debris was pelleted by spinning at 14,000 rpm in a tabletop microcentrifuge for 10 minutes at 4° C. Extracts were moved to a fresh tube and shipped to General Metabolics for assessment by EIS-MS/MS. Differential expression analysis was done using limma.

RNA-Seq Analysis.

Cells were cultured as described in the metabolite profiling by EIS-MS/MS section above. mRNA was extracted from cell lysates using oligo-dT beads (Invitrogen). For cDNA synthesis, We used custom oligo-dT primer with a barcode and adaptor-linker sequence (CCTACACGACGCTCTTCCGATCT-XXXXXXXX-T15)(SEQ ID NO:1). After first-strand synthesis, samples were pooled together based on Actb qPCR values and RNA-DNA hybrids were degraded with consecutive acid-alkali treatment. Subsequently, a second sequencing linker (AGATCGGAAGAGCACACGTCTG)(SEQ ID NO:2) was ligated with T4 ligase (NEB) followed by SPRI-beads (Agencourt AMPure XP, BeckmanCoulter) clean-up. The mixture was enriched by PCR for 12 cycles and SPRI-beads (Agencourt AMPure XP, BeckmanCoulter) purified to yield final strand-specific RNA-seq libraries. Libraries were sequenced using a HiSeq 2500 (Illumina) using 50 bp×25 bp pair-end sequencing. Second read (read-mate) was used for sample demultiplexing. Reads were aligned to the GRCm38.p2 assembly of mouse genome using STAR aligner. Aligned reads were quantified using quant3p script to account for specifics of 3′ sequencing. RefSeq genome annotation was used and DESeq2 was used for differential gene expression analysis. RNAseq data has been deposited at GEO:GSE98563.

Network Analysis.

Network analysis was performed as previously described utilizing Shiny GAM. We considered a network of chemical mappings between carbon atoms in substrates and products for all annotated reactions in KEGG database using RPAIRs entries. The scores for nodes and edges were assigned according to log(p-value), such that highly significant gene or metabolite signals had positive scores and not significant had negative scores. Using an exact solver We found a module with a maximal weight, with counting positive scores maximum once for a measured entity (a mass-spectrometry peak or a gene). For clarity, addition edges between nodes in the module were added if the corresponding gene was highly expressed (was in a top 3000 expressed genes).

qRT-PCR.

Total RNA was isolated with TRIzol Reagent (Invitrogen) and single-strand cDNA was synthesized with a High Capacity cDNA Reverse Transcription Kit (Applied Biosystems). Genomic DNA was extracted using the QIAamp DNA micro kit (Qiagen) to determine mtDNA/nDNA ratios. Real-time PCR was performed using SYBR Green real-time PCR master mix (Thermo-Fisher) and LightCycler 96 detection system (Roche). mtDNA primers were to cytochrome c oxidase subunit 1 and nDNA primers were to NADH:ubiquinone oxidoreductase core subunit V1.

Metabolism Assays.

For real-time analysis of extracellular acidification rates (ECAR) macrophages were analyzed using an XF96 Extracellular Flux Analyzer (Agilent Technologies). Cells were incubated overnight in complete RPMI in the indicated concentration of LCCM with or without cyclocreatine (10 mM). Measurements were taken under basal conditions and following the sequential addition of 1 μM oligomycin and 1.5 μM fluoro-carbonyl cyanide phenylhydrazone (FCCP) (purchased from Sigma-Aldrich).

Transmission Electron Microscopy.

For ultrastructural analyses, cells were fixed in 2% paraformaldehyde/2.5% glutaraldehyde in 100 mM sodium cacodylate buffer, pH 7.2 for 1 hr at RT (Polysciences Inc., Warrington, Pa.). Samples were washed in sodium cacodylate buffer and postfixed in 1% osmium tetroxide for 1 hr (Polysciences Inc.). Samples were then rinsed extensively in deionized water prior to en bloc staining with 1% aqueous uranyl acetate for 1 hr (Ted Pella Inc., Redding, Calif.). Following several rinses in dH₂O, samples were dehydrated in a graded series of ethanol and embedded in Eponate 12 resin (Ted Pella Inc.). Sections of 95 nm were cut with a Leica Ultracut UCT ultramicrotome (Leica Microsystems Inc., Bannockburn, Ill.), stained with uranyl acetate and lead citrate, and viewed on a JEOL 1200 EX transmission electron microscope (JEOL USA Inc., Peabody, Mass.) equipped with an AMT 8 megapixel digital camera and AMT Image Capture Engine V602 software (Advanced Microscopy Techniques, Woburn, Mass.).

For quantitation of multivesicular/multilamellar structures, 30 cells that were cross-sectioned through the nucleus (indicating cross-section through the middle of cell) were randomly chosen, and images of each cell were taken at 6,000× and 20,000× magnification. The cross-sectional area of each of the multivesicular structures were determined using Image J 1.38 g (National Institutes of Health, USA, customized for AMT images). Data is expressed as the 1) total number of a multivesicular/multilammelar structures per cross-sectional area of cytosol and 2) the total cross-sectional area of multivesicular/multilamellar structures per area of cytosol.

Preparation of Brain Samples and Confocal Microscopy.

Confocal microscopy analysis was performed as previously described. Briefly, mice were anesthetized with ketamine/xylazine and perfused with ice-cold PBS containing 1 U/ml of heparin. Brains were fixed in 4% PFA overnight at 4° C. rinsed in PBS and incubated overnight at 4° C. in 30% sucrose before freezing in a 2:1 mixture of 30% sucrose and optimal cutting temperature compound. Serial 40 μm coronal sections were cut on a cryo-sliding microtome. Floating sections from 1.1 mm Bregma to 0.8 mm Bregma for cortical imaging or slides with fixed human sections were stained with Iba-1 (Waco Chemicals Cat. Number 019-19741) overnight at 4° C. followed by staining with anti-rabbit IgG DyLight 549 (Vector Laboratories Cat. Number DI-1549) and methoxy-X04 (Tocris Cat. Number 4920) for 1 hour at RT. Finally, sections were stained for with anti-LC3 Alexa 488±anti-cleaved caspase 3 (Cell Signaling Technologies Cat. Number 13082 and 9602). Images were collected using a Nikon A1Rsi+ confocal microscope. Images were then processed with Imaris 7.7 (Bitplane).

Microglia Clustering Analysis.

Positions of microglia and positions and volumes of plaques within z-stacks were derived from analysis in Imaris, and microglia-plaque association was determined using automated scripts in Matlab. Briefly, each plaque in the z-stack was modeled as an idealized sphere with the same volume and center of mass. Microglia density within 15 μm of the plaque surface was determined by isolating the voxels of the image that fall within 15 μm of the edge of the idealized plaque. The number of microglia contained in these voxels was divided by the total volume of those voxels to obtain density for a single plaque. Densities of all plaques in a z-stack were averaged together, and the resulting values were averaged together for all z-stacks corresponding to a single animal.

Reporter Cell Assay.

The 2B4 T cell hybridoma cell line was retrovirally transduced with an NFAT-GFP reporter construct, and TREM2 reporter cells were generated by a second retroviral transduction with a TREM2 overexpression construct and selected by puromycin resistance, as previously described. Cells were cultured routinely in complete media (10% FBS in RPMI-1640 supplemented with sodium pyruvate, GlutaMAX, and penicillin/streptomycin). For serum starvation, cells were plated at a density of 25,000 cells/well in a 96-well plate in either complete media or RPMI-1640 in the presence of 20% anti-TREM2 hybridoma supernatant (clone M178, generated in house) or 20% isotype control hybridoma supernatant. After 16 hours, the percent of GFP+ cells among live cells was measured by flow cytometry.

Quantification of Methoxy-X04 Coverage.

To measure total plaque area, brain sections were stained with methoxy X04. Images were collected using a Nikon Eclipse 80i microscope. For quantitative analysis, images were converted to 8-bit greyscale and stitched using the “Stitching” plugin in ImageJ. Cortex (−1.1 mm Bregma to 0.8 mm Bregma) and hippocampus (˜−1.7 Bregma to −2.4 Bregma) were determined by manual selection. The threshold of selected images were set at 1.5x mean intensity of the selected area to highlight plaques and analyzed using the “Measure” function in ImageJ to calculate the percent area covered. Identified objects after thresholding were individually inspected to confirm the object as a plaque or not. Two brain sections per mouse were used for quantification. The average of two sections was used to represent a plaque load for each mouse.

Plaque Morphology Analysis.

Methoxy-X04-stained sections were imaged by confocal microscopy using a 60× objective and 1.5× digital zoom in the cortex at ˜1.1 mm Bregma to 0.8 mm Bregma. Z images were taken at 1.2 μm intervals. 20-30 μm z-stacks were z-projected by maximum intensity projection and individual plaques were selected in ImageJ. Each individual plaque was traced using a combination of thresholds and edge detection and smoothened using image erosion. The average intensity was determined by averaging values of pixels within the plaque trace. The shape index was calculated as 4π*(perimeter pixels)²/(all pixels).

Quantification and Statistical Analysis

Data in FIG.s are presented as mean±SEM. Unless otherwise stated statistical analysis was performed using Prism (GraphPad). Quantification of confocal microscopy, immunoblots, and electron microscopy images were performed using Imaris, ImageJ, Matlab, and FIJI. Differential metabolite expression was analyzed using limma. Pathway analysis of microarray data was performed using IPA software. RNAseq analysis was performed by using Second read (read-mate) for sample demultiplexing. Reads were aligned using STAR aligner and quantified using quant3p script. RefSeq genome annotation was used and DESeq2 was used for differential gene expression analysis. Combined RNAseq and metabolite network analysis was performed utilizing Shiny GAM. Statistical analysis to compare the mean values for multiple groups was performed using Prism by one-way ANOVA with Holm-Sidak's multiple comparisons test. Comparison of two groups was performed in Prism using a two-tailed unpaired t-test (Mann Whitney). Values were accepted as significant if P<0.05. Intragroup variation compared between groups was similar in all experiments.

Data and Software Availability

Microarray data has been deposited at GEO:GSE65067.

RNAseq data has been deposited at GEO:GSE98563.

TABLE 2
KEY RESOURCES TABLE

| REAGENT or RESOURCE | SOURCE | IDENTIFI |
|---|---|---|
| Antibodies | | |
| anti-Pan Actin | Cell Signaling Technologies | Cat# 4968 |
| anti-p62 | Cell Signaling Technologies | Cat# 8025 |
| anti-phospho Akt 473 | Cell Signaling Technologies | Cat# 9271 |
| anti-phospho Ulk1 317 | Cell Signaling Technologies | Cat# 12753 |
| anti-phospho Ulk1 757 | Cell Signaling Technologies | Cat# 6888 |
| anti-phospho NDRG1 | Cell Signaling Technologies | Cat# 3217 |
| anti-phospho S6K | Cell Signaling Technologies | Cat# 2710 |
| anti-phospho 4EBP1 | Cell Signaling Technologies | Cat# 2855 |
| anti-Akt | Cell Signaling Technologies | Cat# 9272 |
| anti-S6K | Cell Signaling Technologies | Cat# 9202 |
| anti-phospho AMPKα | Cell Signaling Technologies | Cat# 2535 |
| anti-AMPKα | Cell Signaling Technologies | Cat# 5832 |
| anti-LC3 | Cell Signaling Technologies | Cat# 4108 |
| anti-phospho mTOR 2448 | Cell Signaling Technologies | Cat# 2971 |
| anti-Spp1 | R and D Systems | Cat# AF808 |
| anti-Xbp-1s | BD Biosciences | Cat# 562642 |
| anti-Iba1 | Wako | Cat# 019-19741 |
| anti-cleaved caspase-3 Alexa 647 | Cell Signaling Technologies | Cat# 9602 |
| anti-LC3 488 | Cell Signaling Technologies | Cat# 13082 |
| anti-APP | Milipore | Cat# MAB348 |
| anti-CD45 BV421 | Biolegend | Cat# 103134 |
| anti-CD11b FITC | eBioscience | Cat# 11-0112 |
| anti-F4/80 APC | eBioscience | Cat# 17-4801 |
| anti-Trem2 | Colonna Lab | M1178 doi: |
| | | 10.1016/j.cell.2015.01.049. |
| anti-rabbit DyLight 549 | Vector Laboratories | Cat# DI-1549 |
| Bacterial and Virus Strains | | |
| Murine Trem2 expressed in pMXs- | Cell Biolabs | https://www.cellbiolabs.com/pmxs- |
| IRES-Puro Retroviral Expression Vector | | ires-puro-retroviral-expression-vector |
| Biological Samples | | |
| Human brain tissue for Alzheimer's | Knight Alzheimer's | Protocol numbers: |
| disease patients (TREM2^{CV/CV} (8), | Disease Research Center at | Healthy Aging |
| TREM2^{CV/R47H} (7), TREM2^{CV/R62H} (4)) | Washington University | and Senile Dementia |
| | | (HASD) Morphology |
| | | Core: 89-0555 |
| | | and Program |
| | | Project: |
| | | Alzheimer's |
| | | Disease Research |
| | | Center (ADRC): |
| | | 89-0556. |
| Chemicals, Peptides, and | | |
| Recombinant Proteins | | |
| Cyclocreatine | Santa Cruz Biotechnology | Cat# SC-217964 S |
| Methoxy X04 | Tocris Biosciences | Cat# 4920 |
| MitoTraker Green | Invitrogen | Cat# M7514 |
| Wortmannin | Millipore | Cat# 12-338 |
| Ly294002 | Calbiochem | Cat# 440202 |
| Tunicamycin | Sigma-Aldrich | Cat# 654380 |
| Bafilomycin A1 from Streptomyces | Sigma-Aldrich | Cat# B1793-10UG |
| griseus | | |
| ToPro3 Iodide | Life Technologies | Cat# T3605 |
| Zymosan, depleted | Invivogen | Cat# tlrl-zyd |
| Oligomycin | Cayman Chemical | Cat# 11341 |
| FCCP | Cayman Chemical | Cat# 370865 |
| Critical Commercial Assays | | |
| ATP Assay | Invitrogen | Cat# A22066 |
| Neural Tissue Dissociation Kit (T) | Miltenyi Biotech | Cat# 130-093-231 |
| Deposited Data | | |
| GEO/GSE65067 | Microarray data | |
| GEO/GSE98563 | RNAseq data | |
| Experimental Models: Cell Lines | | |
| 2B4 cells retrovirally transduced with | Generated in the laboratory | doi: |
| an NFAT-GFP reporter and retrovirally | of Dr. Marco Colonna. | 10.1016/j.cell.2015.01.049. |
| transduced with TREM2 | | |
| 2B4 cells expressing NFAT-GFP | Generated in the laboratory | doi: |
| | of Dr. Lewis Lanier | 10.1126/science.1070884 |
| Experimental Models: | | |
| Organisms/Strains | | |
| Mouse: C57BL/6J WT | The Jackson Laboratory | Cat# 000664 |
| Mouse: 5XFAD/Tg6799 | The Jackson Laboratory | Cat# 34840-JAX |
| Mouse: Trem2^{-/-} | Generated in the Laboratory | |
| | of Dr. Marco Colonna | |
| Oligonucleotides | | |
| For nuclear DNA | FW: | |
| | CTTCCCCACTGGCCTCAAG | |
| | (SEQ ID NO: 3) | |
| | RV: | |
| | CCAAAACCCAGTGATCCAGC | |
| | (SEQ ID NO: 4) | |
| For mitochondrial DNA | FW: | |
| | TGCTAGCCGCAGGCATTAC | |
| | (SEQ ID NO: 5) | |
| | RV: | |
| | GGGTGCCCAAAGAATCAGAAC | |
| | (SEQ ID NO: 6) | |
| For beta-Actin (RNAseq) | FW: | |
| | GGA GGG GGT TGA GGT GTT | |
| | (SEQ ID NO: 7) | |
| | RV: | |
| | TGT GCA CTT TTA TTG GTC TCA AG | |
| | (SEQ ID NO: 8) | |
| Linker primers for RNAseq | CCTACACGACGCTCTTCCGATCT- | |
| | XXXXXXXX-T15 | |
| | (SEQ ID NO: 1) | |
| | AGATCGGAAGAGCACACGTCTG | |
| | (SEQ ID NO: 2) | |
| Recombinant DNA | | |
| Software and Algorithms | | |
| Matlab | MathWorks | |
| Morpheus | Broad Institute | |
| Prism 7 | Graphpad | |
| Fiji 2.0 | ImageJ | |
| Imaris 7.7 | Bitplane | |
| IPA | QIAGEN | |
| Shiny GAM | https://artyomovlab.wustl.edu/shiny/gam/ | |
| Other | | |
| XF96 Extracellular Flux Analyzer | Agilent | |
| Nikon A1Rsi Confocal Microscope | Nikon | |

The present disclosure includes a method for treating a microglial dysfunction-associated neurodegenerative disease in a subject in need thereof, the method comprising: administering to the subject a therapeutically effective amount of a composition comprising a microglial rescuing agent, wherein the microglial rescuing agent is one or more of a creatine, a creatine analog, a Dectin-1 agonist or pharmaceutically acceptable salt thereof. In these embodiments, the microglial dysfunction-associated neurodegenerative disease is Alzheimer's disease; the microglial dysfunction-associated neurodegenerative disease is a neurodegenerative disease characterized by SNPs or mutations effecting microglial function, a TREM2 variant, or an ApoE variant, resulting in decreased microglial activity; the microglial dysfunction-associated neurodegenerative disease is characterized by single nucleotide polymorphisms (SNPs) or mutation in Trem2 or ApoE affecting microglial activity; a therapeutically effective amount of a microglial rescuing agent results in one or more of improved microglial metabolic activity, decreased microglial autophagy, reduced neurite dystrophy, decreased cell death, improved microglia viability or improved microglia numbers; the therapeutically effective amount of a microglial rescuing agent results in improved microglia clustering around Aβ plaques or reduced plaque-associated neurite dystrophy; the microglial rescuing agent is one or more of creatine, nicotinamide mononucleotide, cyclocreatine, phosphocyclocreatine, Zymosan, and Zymosan Depleted; and/or the subject is human.

The present disclosure also includes a method of reversing neuronal damage in a subject having a microglial dysfunction-associated neurodegenerative disease, wherein the microglial dysfunction-associated neurodegenerative disease is characterized by a single nucleotide polymorphisms (SNPs) or mutation in Trem2 or ApoE affecting microglial functions, the method comprising: administering to the subject a therapeutically effective amount of a composition comprising a microglial rescuing agent, wherein the microglial rescuing agent is one or more of a creatine, a creatine analog, a Dectin-1 agonist or pharmaceutically acceptable salt thereof. In these embodiments, a therapeutically effective amount of a microglial rescuing agent in one or more of improved microglial metabolic activity, decreased microglial autophagy, reduced neurite dystrophy, decreased cell death, improved microglia viability or improved microglia numbers; the microglial dysfunction-associated neurodegenerative disease is Alzheimer's disease; the therapeutically effective amount of a microglial rescuing agent results in improved microglia clustering around Aβ plaques or reduced plaque-associated neurite dystrophy; the microglial rescuing agent is one or more of creatine, nicotinamide mononucleotide, cyclocreatine, phosphocyclocreatine, Zymosan, and Zymosan Depleted; the subject has TREM2 deficient cells in the brain prior to administration of the microglial rescuing agent; and/or the subject is human.

The present disclosure further includes a method of treating at least one symptom of cognitive dysfunction in a subject having a microglial dysfunction-associated neurodegenerative disease, wherein the microglial dysfunction-associated neurodegenerative disease is characterized by a single nucleotide polymorphisms (SNPs) or mutation in Trem2 or ApoE affecting microglial functions, the method comprising: administering to the subject a therapeutically effective amount of a composition comprising a microglial rescuing agent, wherein the microglial rescuing agent is one or more of a creatine, a creatine analog, a Dectin-1 agonist or pharmaceutically acceptable salt thereof. In these embodiments, at least one symptom comprises short term memory function; at least one symptom comprises a spatial learning dysfunction; the microglial rescuing agent is one or more of creatine, nicotinamide mononucleotide, cyclocreatine, phosphocyclocreatine, Zymosan, and Zymosan Depleted; and/or the subject is human.

### The Protein Tyrosine Kinase Syk is a Critical Driver of Microglial Response to Amyloid β Pathology

Genetic studies in the last few years have highlighted the important role of microglia in controlling the progression of Alzheimer's Disease (AD); currently ongoing extensive efforts focus on elucidating the mechanisms by which microglia impact AD pathology, as well as developing microglia-based immunotherapy that delays progression of AD. Recent metabolic and high dimensional scRNAseq profiling studies have shown that microglia respond to deposition of Aβ plaques—the initial pathologic event in AD—through acquisition of a complex transcriptional profile encompassing four basic signatures, known as disease-associated microglia (DAM), MHC class II microglia, interferon responsive (IFN-R) microglia, and proliferating microglia. It has been previously demonstrated that these microglial responses to AD are partially dependent on TREM2. This activating receptor transmits intracellular signals through immunoreceptor tyrosine-based activation motifs (ITAM) that recruit the protein tyrosine kinase SYK. These observations have prompted the development of AD immunotherapies based on TREM2 activation. Conversely, another AD associated microglial receptor, CD33, inhibits microglia responses by signaling through tyrosine-based inhibitory motifs (ITIM) that recruit SHP1/2, which antagonizes SYK. Together, these observations suggest that an effective microglial response to Aβ relies on a balance of ITAM/ITIM signaling biased towards SYK signaling. Accordingly, as described herein, systemic administration of an antibody that binds CLEC7A, a receptor that directly activates SYK, rescued microglia responses in mice that express the defective TREM2R47H allele and accumulate Aβ plaques. Thus, SYK dominantly drives microglia responses to Aβ and options for AD immunotherapy include agonists for CLEC7A and other microglia receptors that activate SYK.

The present disclosure demonstrates that SYK is a major driver of microglia responses to Aβ pathology irrespective of the cell surface receptors that modulate it. This conclusion is based on the following findings: (1) SYK-deficient microglia are unable to encase Aβ aggregates, resulting in heightened AB plaque pathology and accelerated behavioral deficits; (2) biochemical and imaging analyses show that SYK deficiency impairs the PI3K-AKT signaling pathway, leading to a defect in mammalian target of rapamycin (mTOR) signaling that provides energetic and anabolic support for microglia activation; (3) this metabolic defect is associated with the activation of the autophagic catabolic pathway; (4) impaired metabolic activation impacts microglia acquisition of DAM, MHCII, and IFN-R signatures; and (5) in contrast to TREM2-deficiency, SYK-deficiency allows the expansion of a transitional population of microglia prodromal to DAM, which produces ApoE.

Altogether, the present disclosure provides the first demonstration that SYK is a crucial driver of microglia responses to Aβ, while suggesting the existence of a SYK independent pathway that controls ApoE, an important player in AD pathogenesis.

Importantly, it is further herein demonstrated that this divulged role of SYK can be exploited for AD therapy. Systemic administration of a monoclonal antibody that engages CLEC7A (also known as Dectin 1), a receptor that directly recruits and activates SYK, rescues microglia activation and association with AB plaques in a mouse model of AD carrying a hypofunctional mutant of TREM2 incapable of activating SYK and microglial responses to AB plaques. In parallel with the mouse data, both CLEC7A and SYK are highly expressed in microglia in AD by examining a snRNA seq database of post-mortem specimens from AD patients. The present disclosure advances options for immunotherapy of AD, providing a foundation for the use of agonists for CLEC7A and, in general, for a broad range of microglia receptors that activate ITAM pathways converging on SYK.

Summary

Genetic studies have highlighted a role for microglia in orchestrating Alzheimer's disease (AD). Microglia that adhere to Aβ plaques acquire a complex transcriptional profile, “disease-associated microglia” (DAM), which is partially dependent on the receptor complex TREM2-DAP12 that transmits intracellular signals through the protein tyrosine kinase SYK. The human TREM2^{R47H} variant associated with high AD risk fails to activate microglia via SYK. As described herein, it was found that SYK-deficient microglia cannot encase Aβ plaques, resulting in accelerated brain pathology and behavioral deficits. SYK deficiency impaired the PI3K-AKT-mTOR pathway, affecting energetic and anabolic support required for acquisition of the DAM profile. Systemic administration of an antibody that binds CLEC7A, a receptor that directly activates SYK, rescued microglia responses in mice that express the defective TREM2^{R47H} allele and accumulate Aβ plaques. Thus, SYK dominantly drives microglia responses to Aβ and options for AD immunotherapy include agonists for CLEC7A and other microglia receptors that activate SYK.

Introduction

Alzheimer's disease (AD) is the leading cause of late-onset dementia among the elderly, affecting millions of individuals worldwide (2021). AD pathology initiates with the formation of extracellular aggregates of amyloid beta (Aβ) peptides in the brain, which derive from the processing of amyloid precursor protein (APP) by BACE1 and -g-secretase. Aβ deposits elicit hyperphosphorylation of neuronal tau, which forms intracellular cytotoxic aggregates that cause neuron and synapses loss, ultimately leading to cognitive impairment. Genetic studies on familial early-onset AD and sporadic late-onset AD have identified multiple loci and genes associated with AD risk and functional characterization has unveiled potential cellular and molecular mechanisms involved in AD etiology. While genes associated with familial AD support the involvement of the amyloidogenic processing of APP, many candidate genes associated with late-onset AD have pinpointed a contribution of microglia. Microglia are professional phagocytes of the central nervous system (CNS) that contribute to CNS development, homeostasis and defense against acute and chronic injuries. In Aβ-induced pathology, microglia proliferate, associate with Aβ plaques, and acquire an activated transcriptional profile, commonly referred to as disease-associated microglia (DAM). This type of microglia response has been reported in multiple studies, and represents a specific transcriptional state of microglia that are in proximity to amyloid deposits (plaque-associated microglia). With the increasing resolution power of single-cell RNA sequencing (scRNA-seq) technologies, DAM complexity has been further resolved, identifying four basic signatures of microglia during amyloid pathology, namely DAM proper (enriched for ApoE, Spp1, Clec7a, Cd11c, Csf1, Lyz2, Cst7, Lpl), MHC class II (MHCII) microglia (enriched for MHC-II molecules and Cd74), type I interferon responsive (IFN-R) microglia (enriched for Ifitm3, Bst2, Isg15, Stat1), and proliferating microglia (enriched for Mki67 and Top2a). It has been suggested that plaque-associated microglia may help reduce Aβ toxicity and delay disease progression. However, it is possible that sustained and excessive microglia activation may in fact aggravate neuronal damage at later stages of amyloid pathology. To better comprehend beneficial and detrimental functions of plaque-associated microglia, it is necessary to identify the underlying mechanisms of DAM generation.

One of the late onset AD candidate genes encodes the microglia receptor TREM2, which associates with the transmembrane adaptor DAP12 that contains cytoplasmic immunoreceptor tyrosine-based activation motifs (ITAM). These motifs recruit the non-receptor spleen tyrosine kinase (SYK), which promotes tyrosine phosphorylation and activation of multiple downstream mediators. Hypofunctional variants of TREM2 associated with high AD risk, such as TREM2^{R47H}, affect ITAM signaling, which impairs the capacity of microglia to acquire DAM, MHCII, IFN-R, and proliferating signatures; consequently, microglia fail to restrict spreading of Aβ plaques in a mouse model of Aβ aggregation. A similar phenotype was observed in Trem2^{-/-} mice. Conversely, another microglia receptor associated with AD, CD33, contains cytoplasmic immunoreceptor tyrosine-based inhibitory motifs (ITIMs), which recruit the protein tyrosine phosphatases SHP1 and SHP2 that antagonize the SYK pathway. Human CD33 gain-of-function variants increase AD risk, whereas CD33-deficiency in mouse attenuates Aβ pathology. Together, these observations suggest that an effective microglial response to Aβ relies on a balance of ITAM/ITIM signaling biased towards SYK signaling. Further supporting this concept, microglia upregulate the expression of additional receptors during the acquisition of the DAM profile, such as CD300LB, CD200R4, and CLEC7A, which activate the ITAM pathway either by signaling through DAP12 (CD300LB, CD200R4) or by directly recruiting SYK (CLEC7A). Therefore, as described herein, SYK signaling plays a central role in microglial responses to Aβ.

To directly test the impact of SYK signaling in microglia response to AD pathology irrespective of the cell surface receptors that activate or inhibit SYK, progression of Aβ pathology was examined in the 5xFAD mouse model of Aβ aggregation carrying a conditional deletion of Syk in microglia. Results demonstrated that SYK-deficient microglia are unable to enclose Aβ aggregates, resulting in increase of Aβ plaque pathology and accelerated behavioral deficits. Biochemical and imaging analyses demonstrated that SYK deficiency impairs the PI3K-AKT signaling pathway, leading to a defect of mammalian target of rapamycin (mTOR) signaling that provides energetic and anabolic support to microglia activation. This metabolic defect was associated with activation of the autophagic catabolic pathway. Impaired metabolic activation impacted the microglia transcriptional profile at the single cell level, affecting the acquisition of DAM, MHCII, and IFN-R signatures. In contrast to TREM2-deficiency, SYK-deficiency did not affect microglia proliferation and featured the accumulation of a transitional state of microglia enriched for ApoE but prodromal to DAM, intimating a SYK independent pathway in response to Al. Importantly, systemic administration of a monoclonal antibody that binds CLEC7A was found to rescue microglia activation and responses to Aβ plaques in 5xFAD mice with hypofunctional TREM2 (TREM2^{R47H}). The present disclosure demonstrates that microglia require SYK signaling to mount an innate immune response towards Aβ, and lack of SYK in microglia aggravates multiple aspects of amyloid pathology. Immunotherapies aiming to activate ITAM-SYK pathways in microglia (such as anti-TREM2 or anti-CLEC7A antibodies) can be considered therapeutic avenues for AD.

Results

Conditional Syk Deletion Blunts Microglial Response to Amyloid Pathology and Exacerbates Plaque Deposition

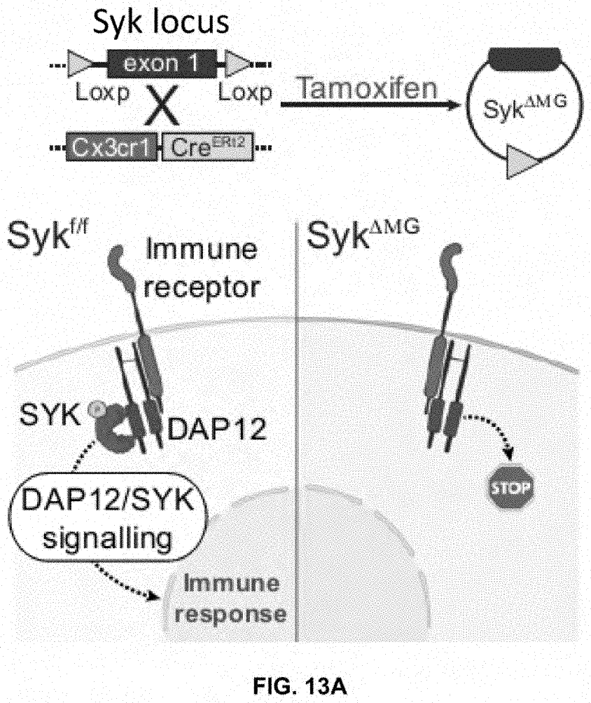

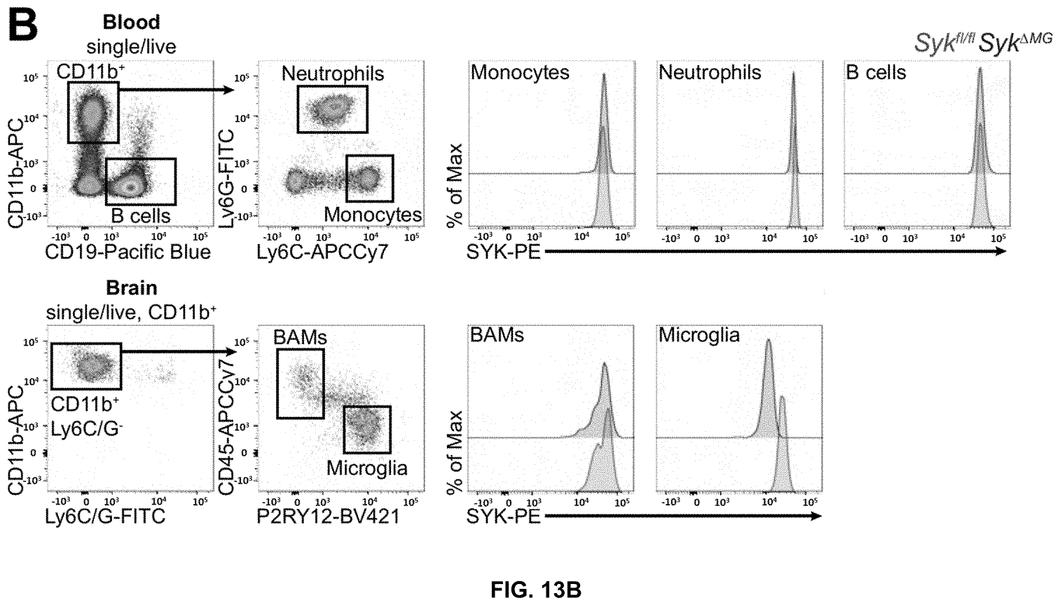

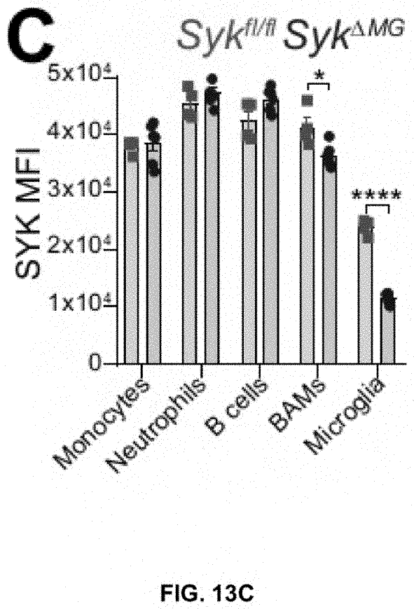

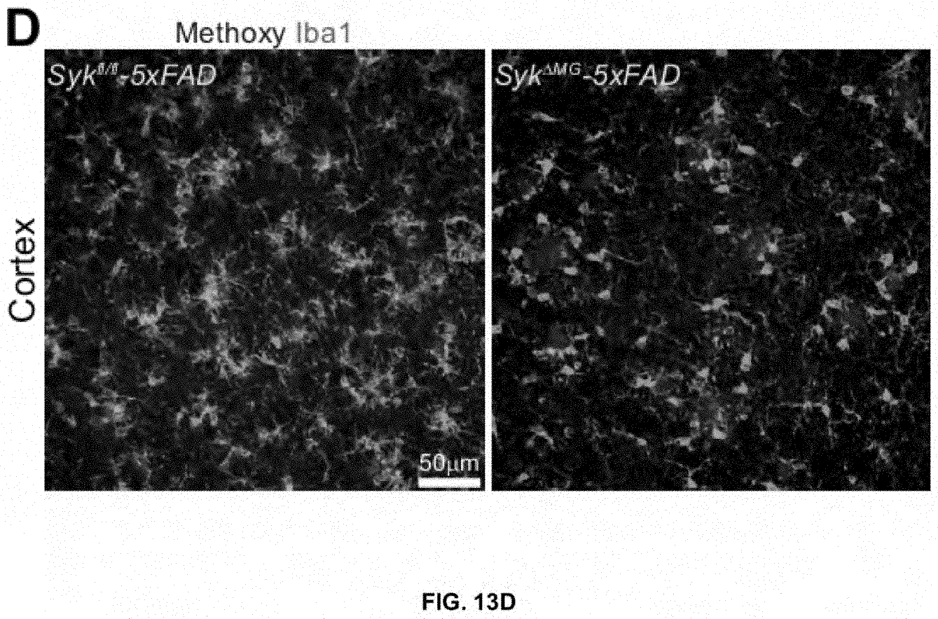

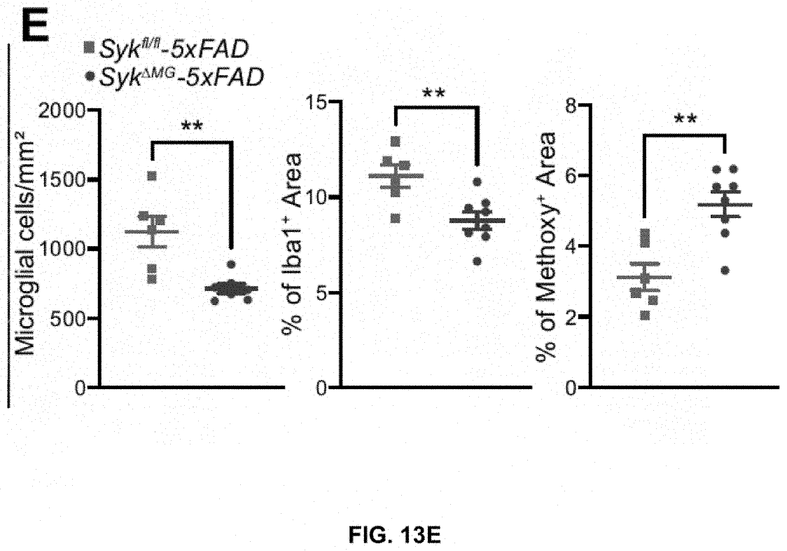

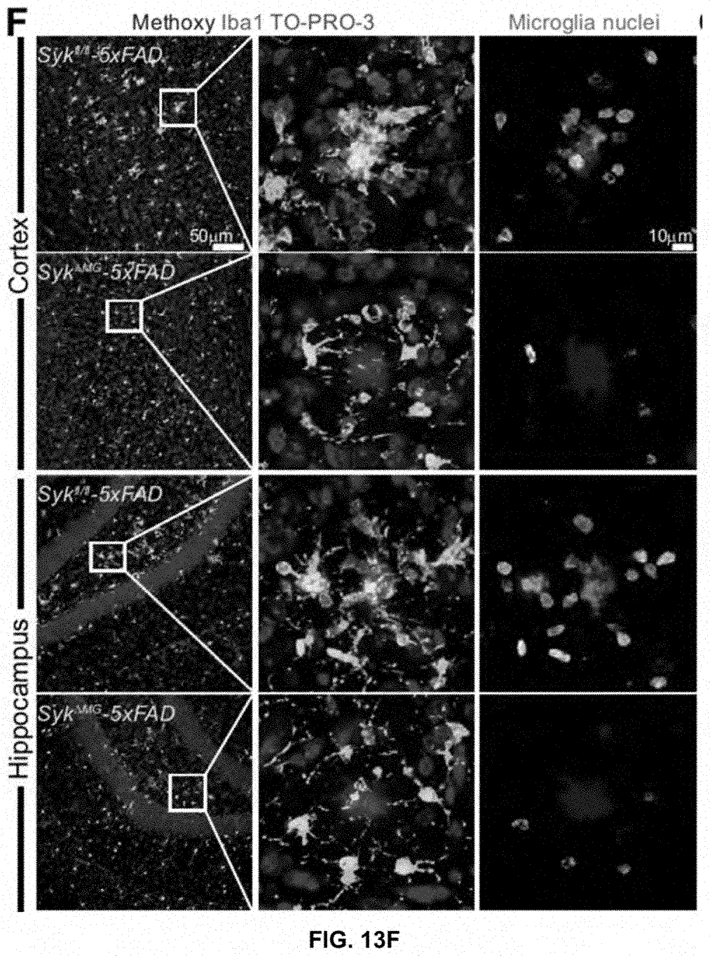

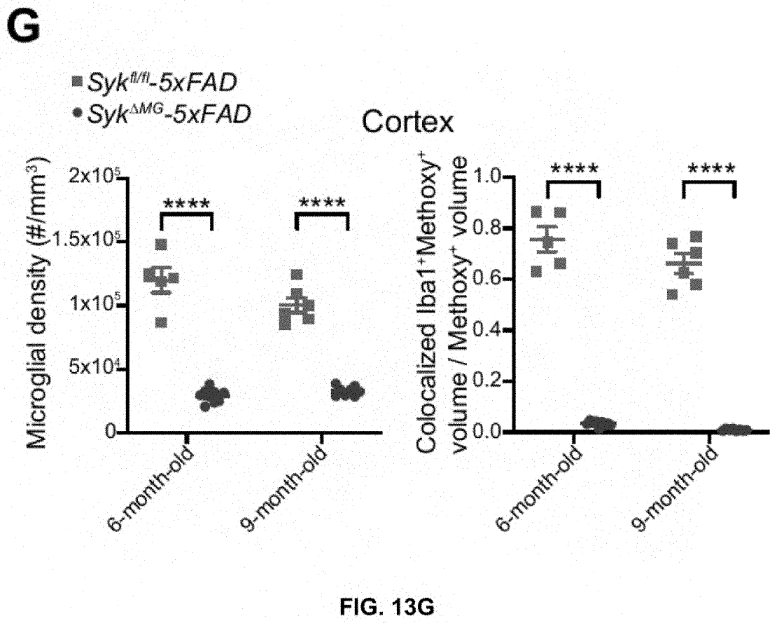

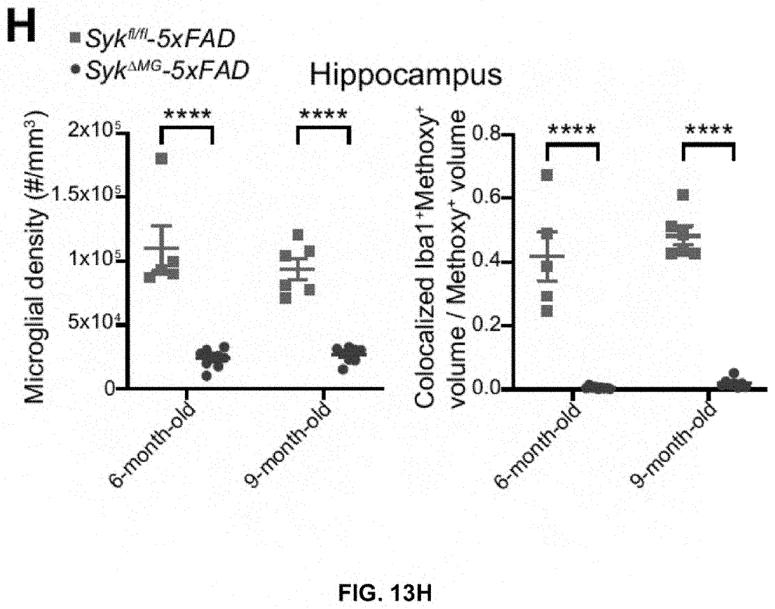

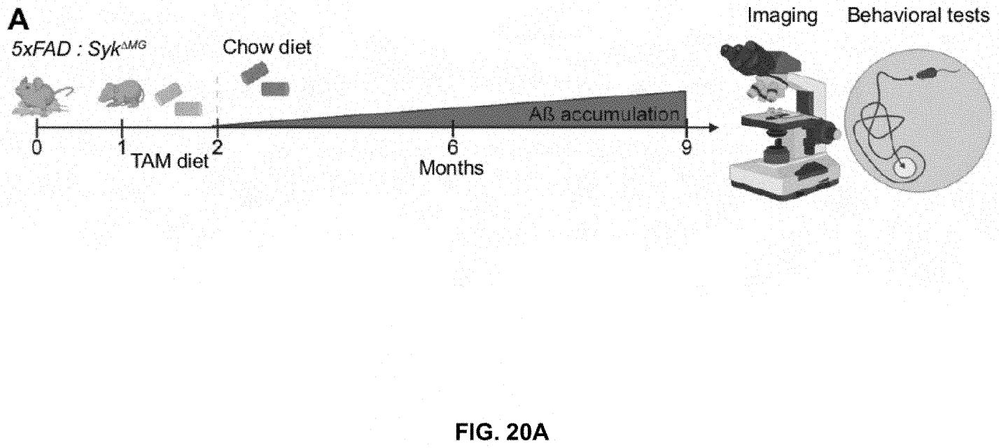

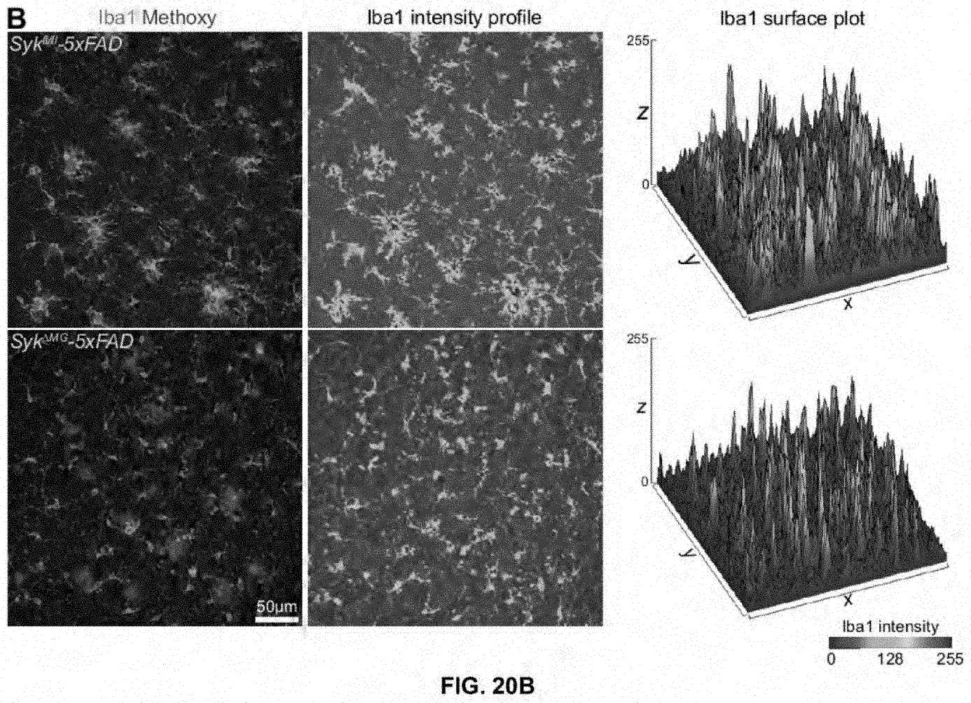

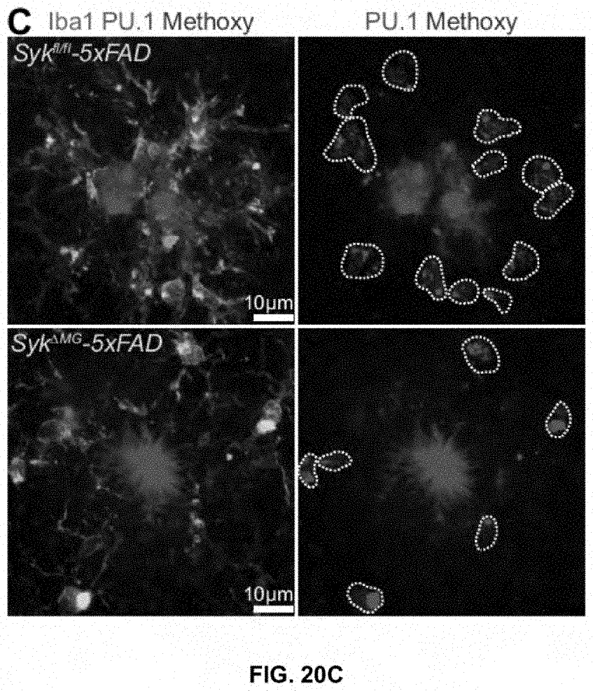

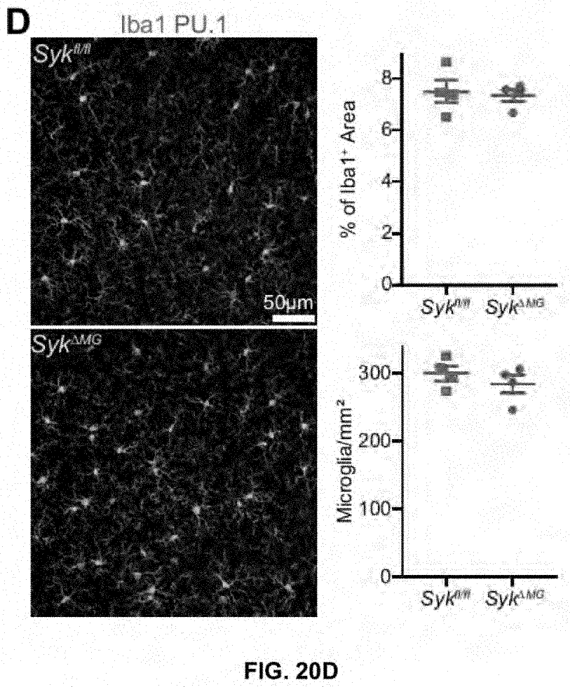

To examine the impact of SYK on microglia response to Aβ pathology, Sykfl/fl mice were crossed with Cx3crl^{CreERT2} mice, which enable inducible deletion of Syk in Cx3crl positive cells by administration of tamoxifen. These mice were referred to as Syk^{ΔMG}, whereas Syk_{fl/fl} served as controls (FIG. 13A). Microglia are long-lived and self-renewing CNS macrophages, hence their maintenance does not rely on circulating monocytes. Thus, this scheme allows for permanent gene deletion in microglia, while recombinant bone marrow-derived monocytes are completely replaced after 4 weeks due to their constant turnover. Using flow cytometry, SYK protein expression was greatly reduced in microglia from Syk^{ΔMG} mice, while leaving monocytes, neutrophils, and B cells unaffected (FIG. 13B, FIG. 13C). Border-associated macrophages (BAMs) exhibited a partial SYK reduction, which is consistent with their embryonic origin and slow turnover. Syk^{ΔMG} mice were further crossed to 5xFAD transgenic mice, which recapitulated features of Aβ pathology characteristic of AD. One-month-old Syk^{ΔMG}-5xFAD mice and Syk^{fl/fl}-5xFAD control mice were fed with tamoxifen-containing chow until two months of age, returned to regular chow and aged up to 6 or 9 months to allow the accumulation Aβ plaques. Both groups were then analyzed by imaging and behavioral testing (FIG. 20A). Nine-month-old Syk^{ΔMG}-5xFAD mice had fewer, more compact microglia (evinced by Iba1 staining) and larger patches stained by methoxy-X04+ indicative of fibrillar Aβ deposition than did Syk^{fl/fl}-5xFAD mice (FIG. 13D, FIGS. 13E, and 20B). The interaction between microglia and Aβ plaques was further examined in these mice (FIG. 13F). In both the cortex and hippocampus, microglia density within a 15 μm radius from plaque borders was significantly reduced in Syk^{ΔMG}-5xFAD mice compared to Syk^{fl/fl}-5xFAD mice. Likewise, Iba1+ methoxy-X04+ colocalized staining was notably diminished in Syk^{ΔMG}-5xFAD mice (FIG. 13G, FIG. 13H). This finding was further confirmed by staining for the transcription factor PU.1, which is expressed solely in microglia in the brain parenchyma (FIG. 20C). However, no difference in microglia numbers or Iba1 staining was found in comparing Syk^{ΔMG} and Syk_{fl/fl} mice without amyloid pathology (FIG. 20D). These data indicate that SYK signaling is required to facilitate microglia clustering around amyloid plaques.

Lack of SYK in Microglia During Amyloid Pathology Correlates with Increased Neuronal Damage and Accelerated Behavioral Deficits

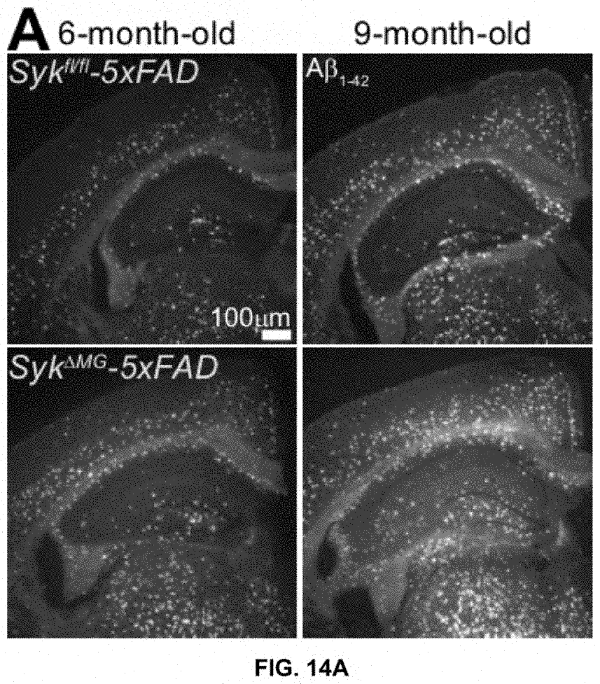

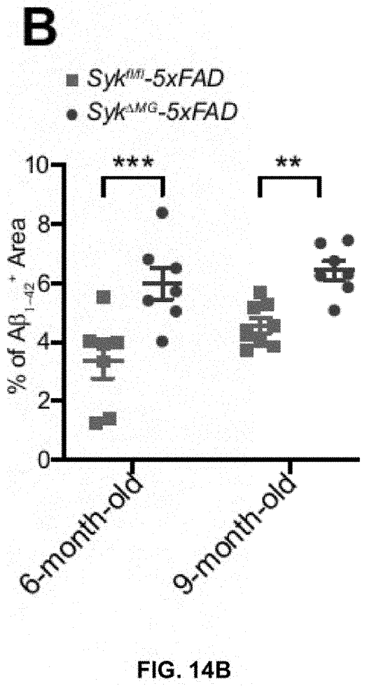

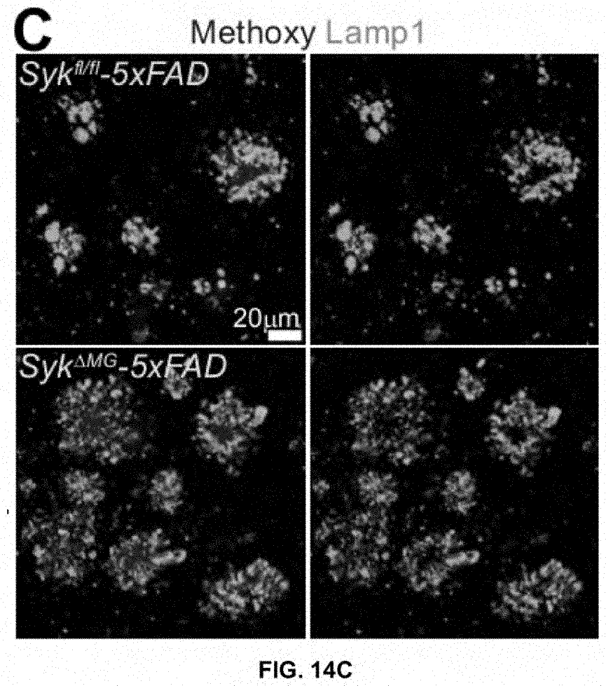

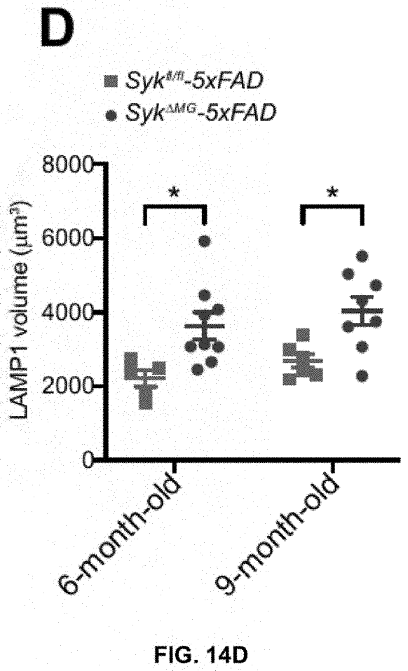

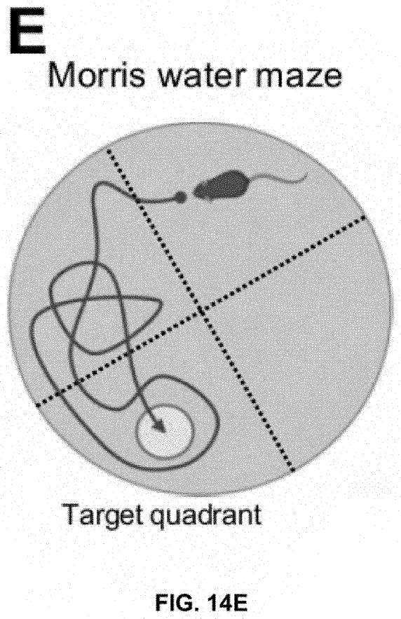

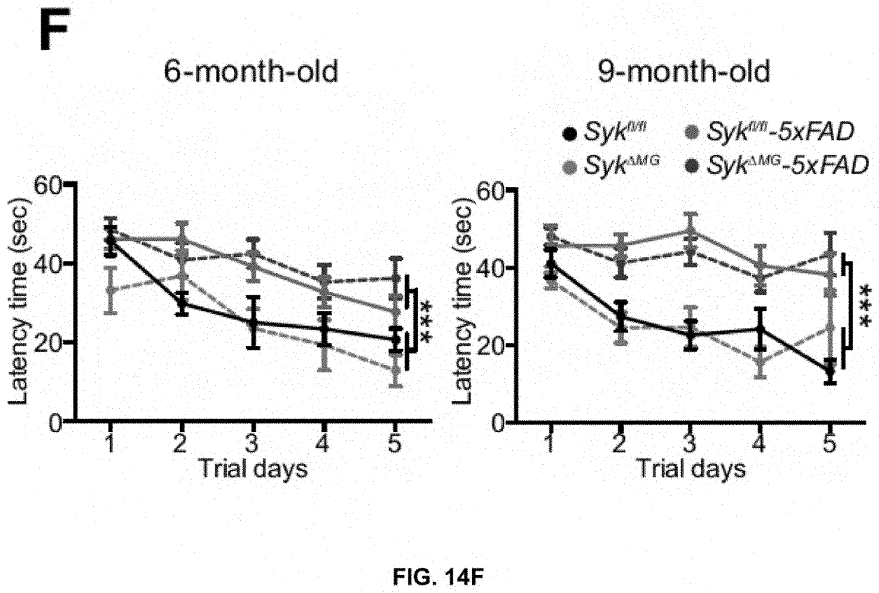

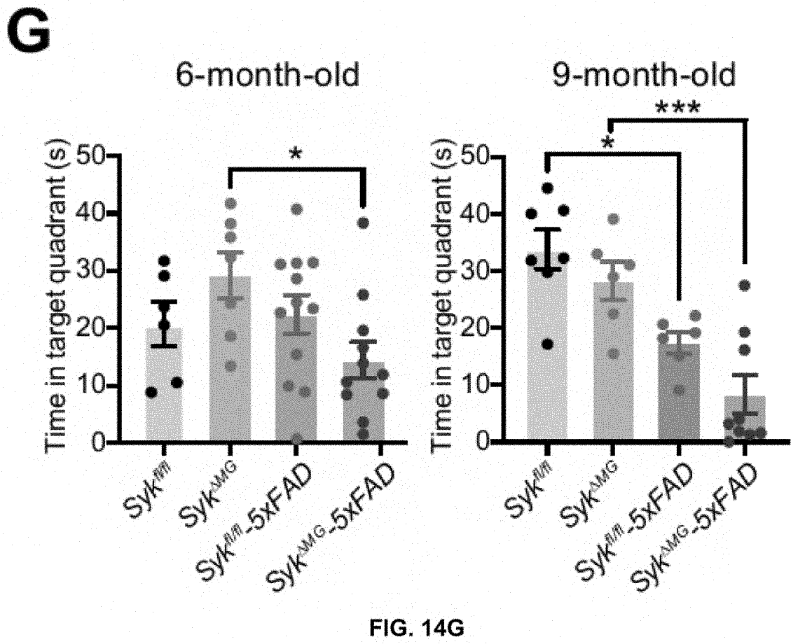

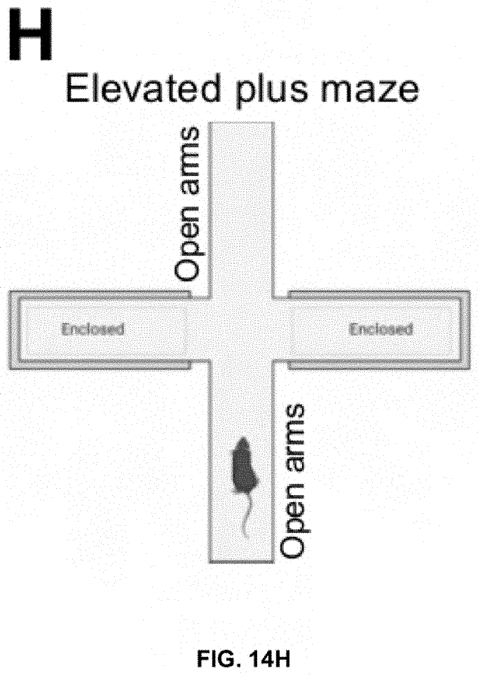

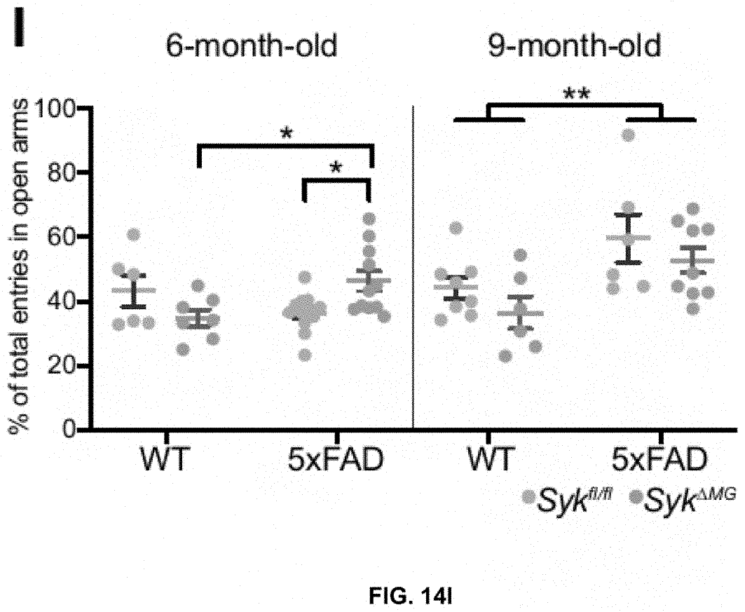

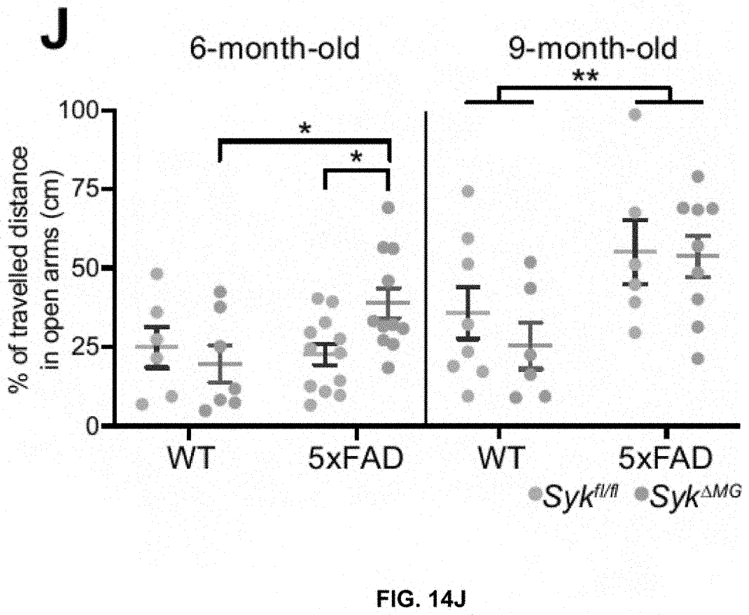

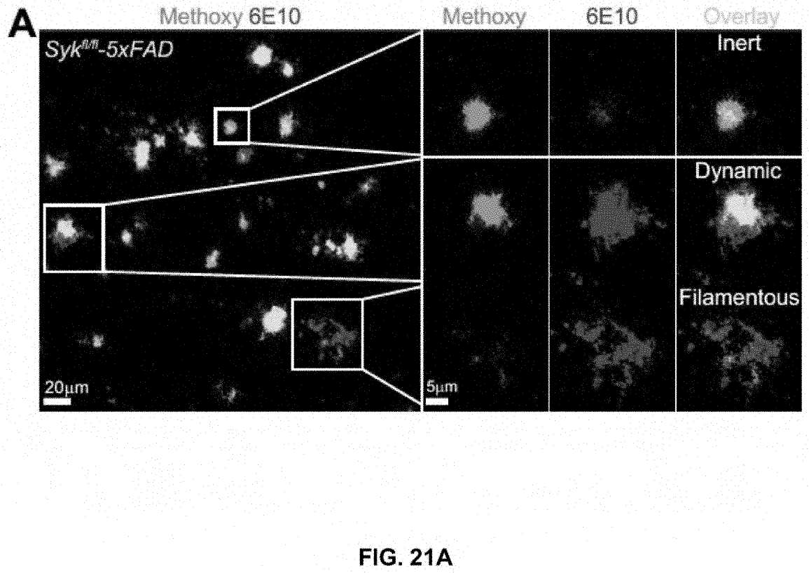

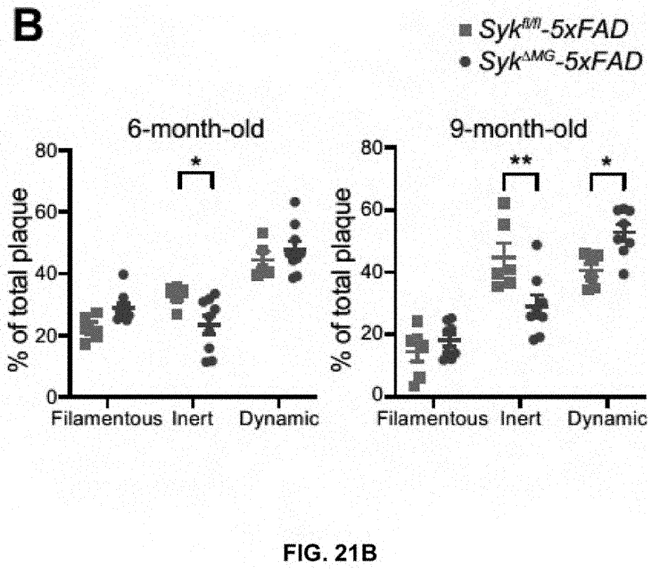

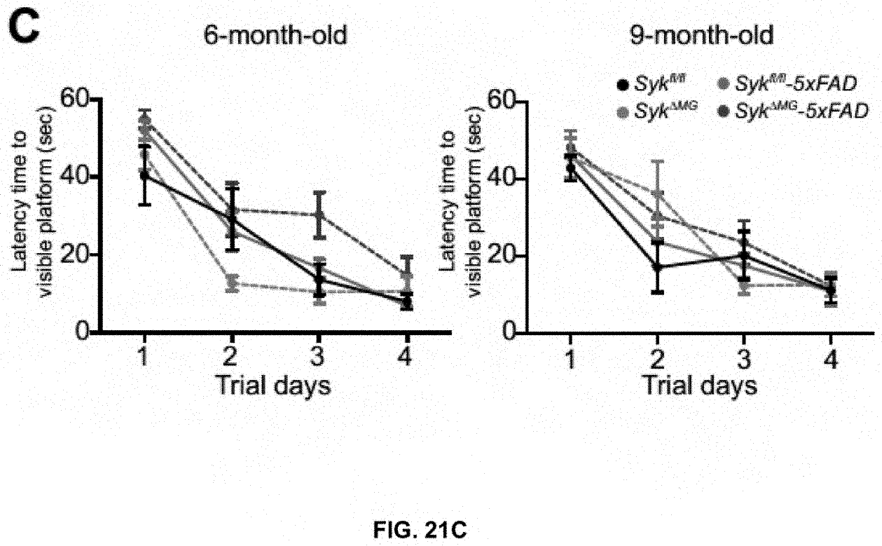

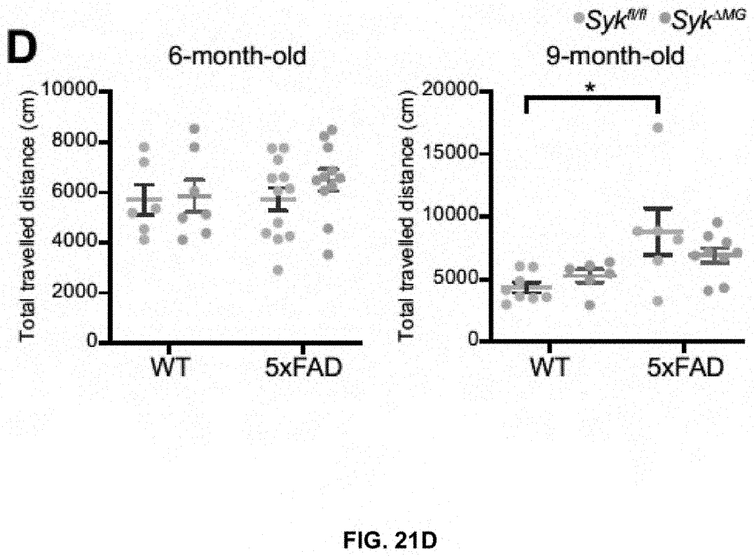

It has been previously suggested that microglia clustering restrains plaque spreading and toxicity, playing a protective function during amyloid pathology. Syk^{ΔMG}-5xFAD and Syk^{fl/fl}-5xFAD mice were compared herein in terms of neuronal damage and behavioral alterations. First, brain accumulation of Aβ₁₋₄₂ was examined by immunofluorescence and found that Syk^{ΔMG}-5xFAD mice had a significant increase of Aβ₁₋₄₂ deposition at both 6 and 9 months (FIG. 14A, FIG. 14B). Aβ₁₋₄₂ is exceptionally prone to aggregation and adhesion to cell membranes due to the hydrophobic C-terminus and it is therefore considered a highly neurotoxic Aβ species. Next, the damage of axons and dendrites (collectively called neurites) was examined by staining for LAMP1, which labels vesicles accumulating in dystrophic neurites. Analysis showed that LAMP1 staining volume was significantly increased in Syk^{ΔMG}-5xFAD mice compared to Syk^{fl/fl}-5xFAD mice (FIG. 14C, FIG. 14D), indicating augmentation of dystrophic neurites. Further, SYK deficiency in microglia impacted the shape and compactness of Aβ plaques. The following were evaluated: 1) “inert plaques” made of fibrillar Aβ mostly stained by methoxy-X04; 2) “dynamic plaques” exhibiting a core of methoxy-X04+ fibrillar Aβ surrounded by a halo of 6E10+ non-fibrillar Aβ; and 3) “filamentous plaques” with little content of 13-sheet structure and branched amyloid fibrils mainly stained with the 6E10 antibody (FIG. 21A). Syk^{ΔMG}-5xFAD mice had fewer inert plaques and more dynamic plaques at 9 months of age than did Syk^{′l/′l}-5xFAD mice (FIG. 21B).

Lastly, a battery of behavioral tests were performed. Morris water maze (MWM) is the gold standard test to evaluate spatial learning and memory retention in mouse (FIG. 14E). Analysis showed that 5xFAD mice expended increased latency time to reach the submerged platform than did the non-5xFAD group at both 6 and 9 months of age (FIG. 14E, FIG. 14F), indicating impaired learning ability. However, no difference was found in comparing Syk^{′l/′l} and Syk^{ΔMG} groups at both ages (FIG. 14E, FIG. 14F). All groups used comparable latency time to reach the visible platform, indicating no impairment of visual or swimming ability (FIG. 21C). Twenty-four hours after the last training day, mice underwent a test session without a submerged platform to assess memory recall. At 6 months, Syk^{ΔMG}-5xFAD mice spent significantly reduced swimming time in the target quadrant than did Syk^{ΔMG} mice, whereas no difference was noted between Syk^{′l/′l}-5xFAD and Syk_{′l/′l} mice (FIG. 14G). At 9-months, both Syk^{ΔMG}-5xFAD and Syk^{′l/′l}-5xFAD groups spent less time in the target quadrant than did either Syk^{ΔG} or Syk_{′l/′l} mice, with Syk^{ΔMG}-5xFAD mice still exhibiting the most significant defect (FIG. 14G). These data indicate that SYK-deficiency in microglia causes accelerated memory impairment in 5xFAD mice.

Mice were also tested at the elevated plus maze (EPM), which assesses anxiety levels (FIG. 14H). At 6 months, Syk^{ΔMG}-5xFAD made significantly more entries and traveled further distance in the open arms of the maze than did Syk^{fl/fl}-5xFAD mice (FIG. 14I, FIG. 14J). At 9 months, however, no difference was found comparing the Syk_{fl/fl} and Syk^{ΔMG} groups, while both 5xFAD groups made more entries and traveled further distance in the open arms than did the non-5xFAD groups (FIG. 14I, FIG. 14J). All groups travelled a comparable total distance, with a slight increase in Syk^{fl/fl}-5xFAD mice at 9 months, indicating no locomotor impairment (FIG. 21D). Overall, these data suggest that SYK deficiency in microglia exacerbates the Aβ₁₋₄₂ load and neurite damage, worsening memory deficits and behavioral alterations.

SYK Deficiency in Microglia Reduces mTOR Signaling and Enhances Compensatory Autophagy

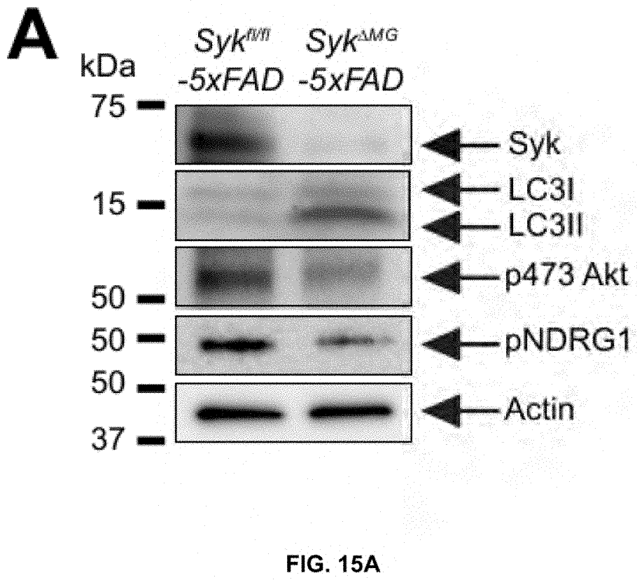

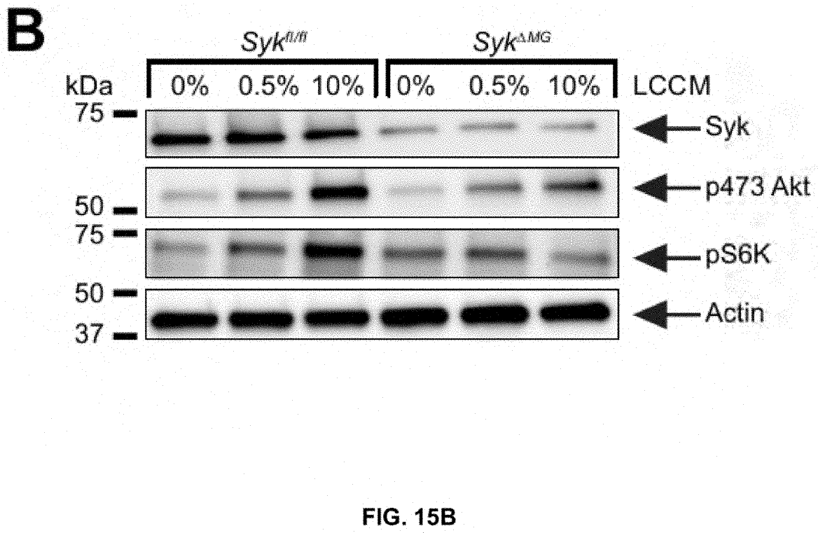

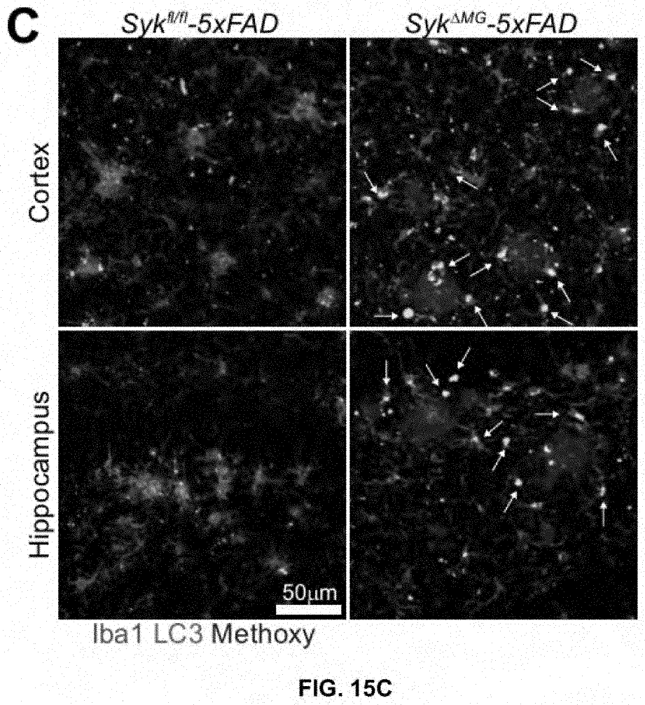

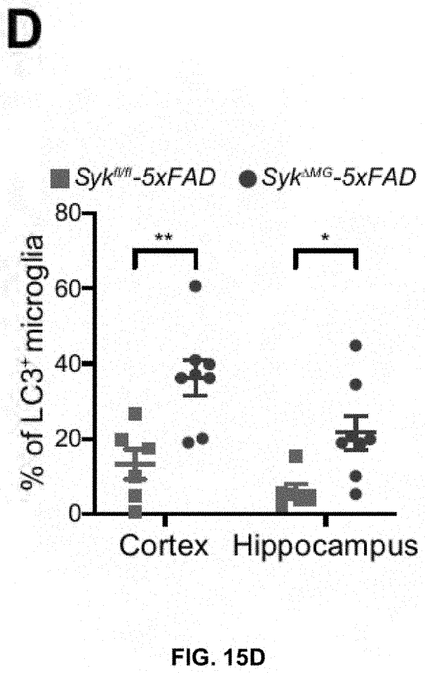

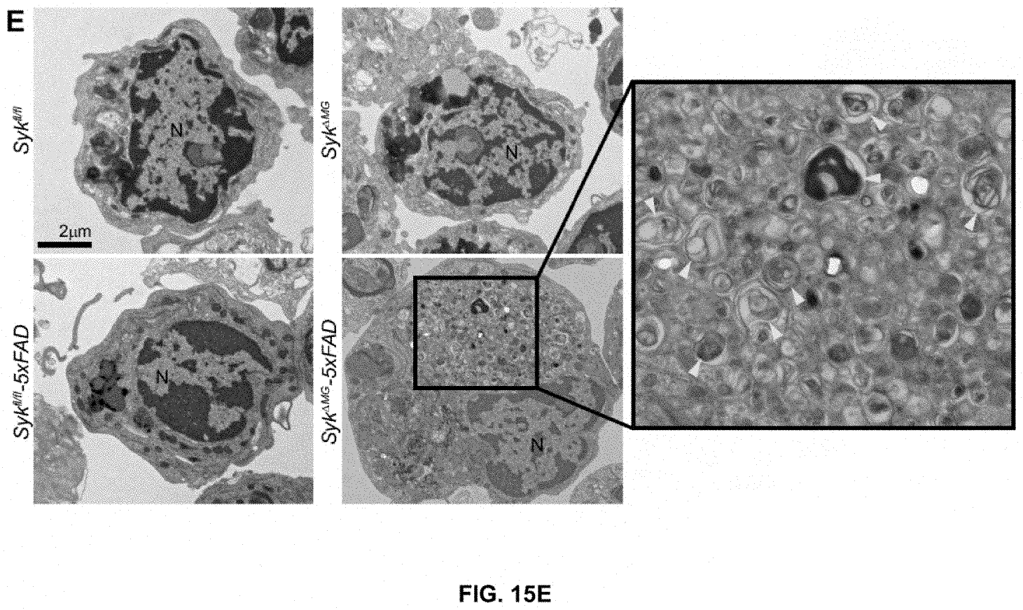

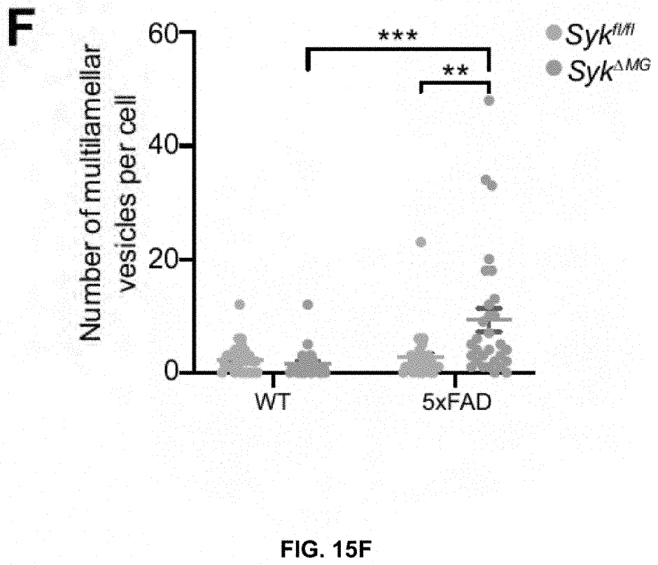

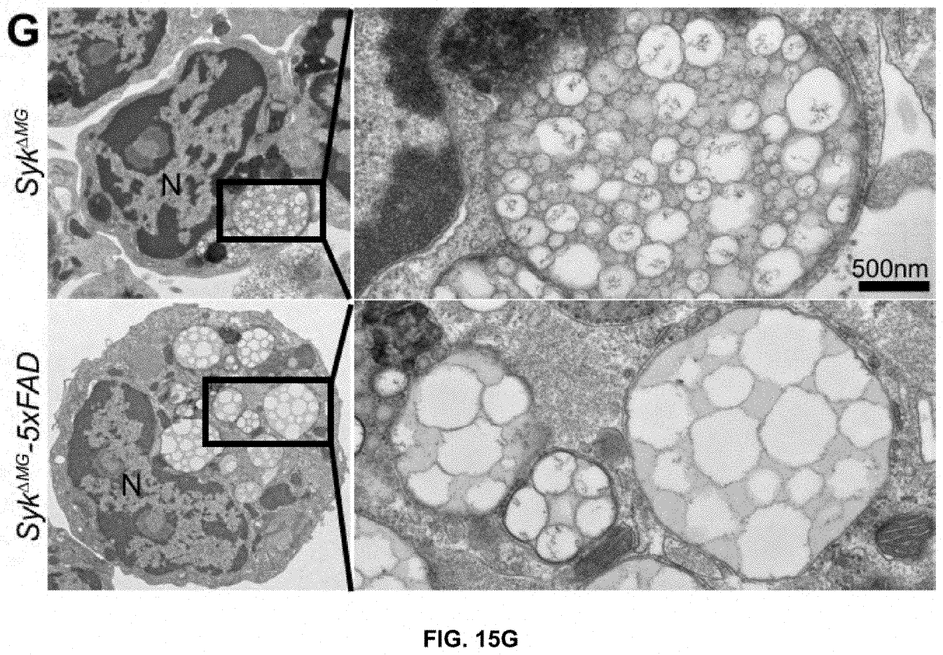

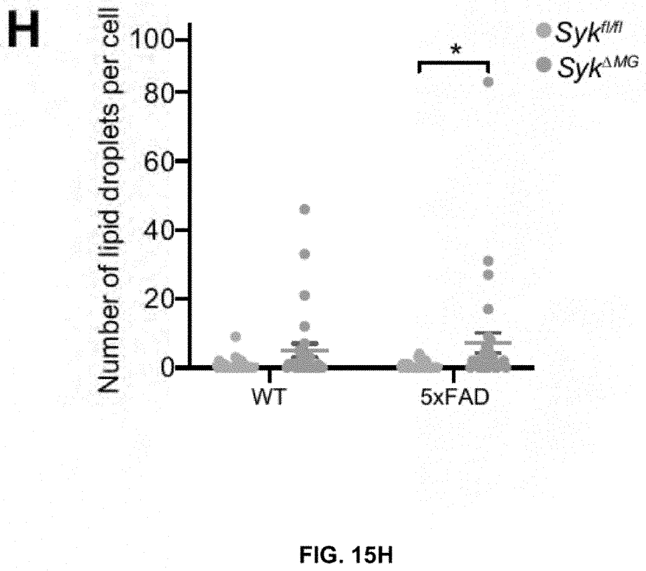

Lack of TREM2-DAP12 impairs PI3K-AKT-mTOR signaling pathway, damping energy production and anabolic pathways required for microglia activation and effector functions. Thus, it was asked whether SYK deficiency acts through a similar mechanism. First, biochemical analyses were performed on microglia sorted from Syk^{ΔMG}-5xFAD and Syk^{fl/fl}-5xFAD mice. SYK deficiency was associated with reduced phosphorylation of AKT and the mTOR target N-myc downstream regulated 1 (NDRG1) (FIG. 15A). In parallel, the ratio of lipidated LC3II to non-lipidated LC3I, which is indicative of autophagy, was markedly higher in Syk^{ΔMG}-5xFAD than in Syk^{fl/fl}-5xFAD microglia (FIG. 15A). Then, in vitro experiments were performed using SYK-sufficient and SYK-deficient thioglycolate-elicited peritoneal macrophages (PM), which were stimulated for 10 min in concentrations of CSF1-containing L cell-conditioned medium (LCCM) ranging from limiting to optimal (0%, 0.5%, 10%) to evaluate sensitivity to metabolic stress. PM were obtained from Syk^{fl/fl}-Cx3crl^{CreERT2} and Syk^{fl/fl} mice injected intraperitoneally with tamoxifen prior to thioglycolate. The defect of SYK in PM was confirmed from Sye^{fl/fl}-Cx3crl^{CreERT2} mice: this defect was associated with a clear reduction of phospho-AKT and ribosomal phospho-S6 Kinase (S6K, a prototypic mTOR target) (FIG. 15B). Notably, the difference was evident at optimal CSF1, indicating a marked metabolic defect. Together, these data suggest that SYK-deficient microglia suffer impaired mTOR signaling and compensate for the energetic demand via activation of catabolic pathways. To validate this conclusion, brain sections were examined by confocal microscopy for the presence of LC3+ puncta, which denote autophagosomes decorated by lipidated LC3II. Many LC3+ puncta were evident in Syk^{ΔMG}-5xFAD microglia, whereas LC3+ puncta were sparse in Syk^{fl/fl}-5xFAD microglia (FIG. 15C, FIG. 15D). To further demonstrate that LC3 puncta reflected ongoing autophagy, the ultrastructural details of microglia were examined by transmission electron microscopy (TEM). Microglia from Syk^{ΔMG}-5xFAD mice contained abundant multivesicular/multilamellar structures indicative of autophagosomes, which were largely absent in microglia from Syk^{fl/fl}, Syk^{ΔMG}, and Syk^{fl/fl}-5xFAD mice (FIG. 15E and FIG. 15F).

Metabolic derailment of SYK-deficient microglia was not limited to constrained mTOR signaling and amplified autophagy. In fact, TEM showed that Syk^{ΔMG}-5xFAD microglia accumulate of lipid-rich vesicles, whereas Syk^{fl/fl}-5xFAD microglia do not (FIG. 15G, FIG. 15H). The accumulation of lipid droplets in microglia indicate limited capacity to degrade membrane debris derived from apoptotic cells or damaged myelin. Additionally, SYK efficiency may impair cholesterol efflux and lipid metabolism, a defect recently observed in TREM2-deficient microglia. Taken together, these data suggest that SYK-deficient microglia exhibit metabolic defects during the development of Aβ pathology, such as impaired PI3K-AKT-mTOR signaling, along with a compensatory increase of autophagy.

SYK is Required for Maintenance of Microglial Clustering Around the Aβ Plaques

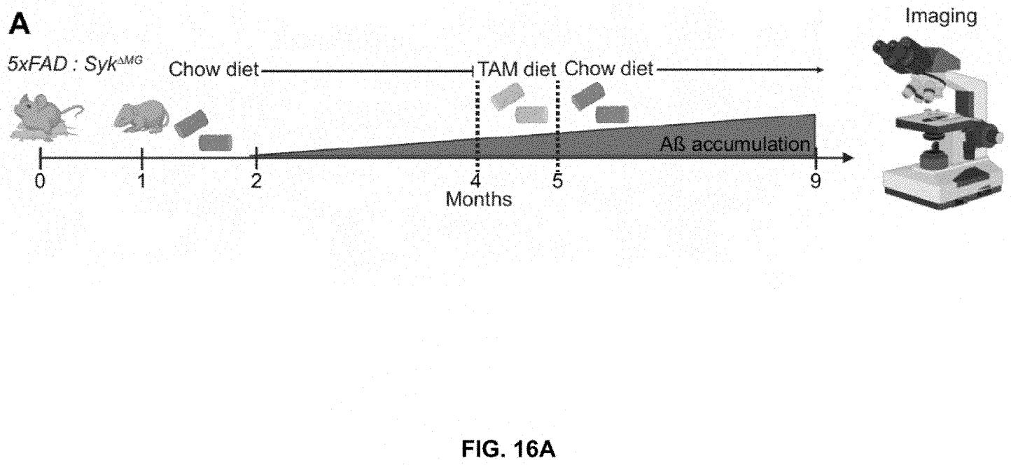

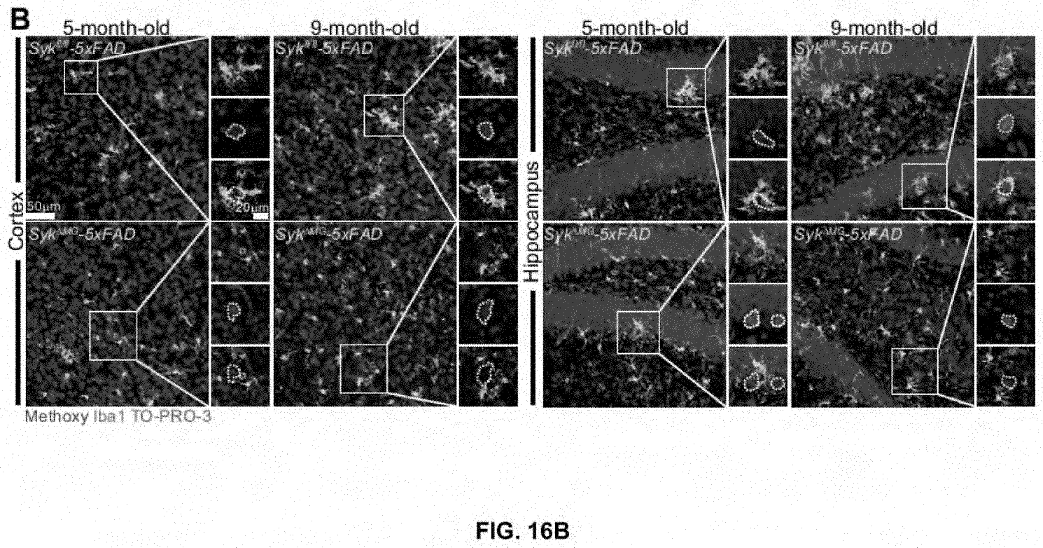

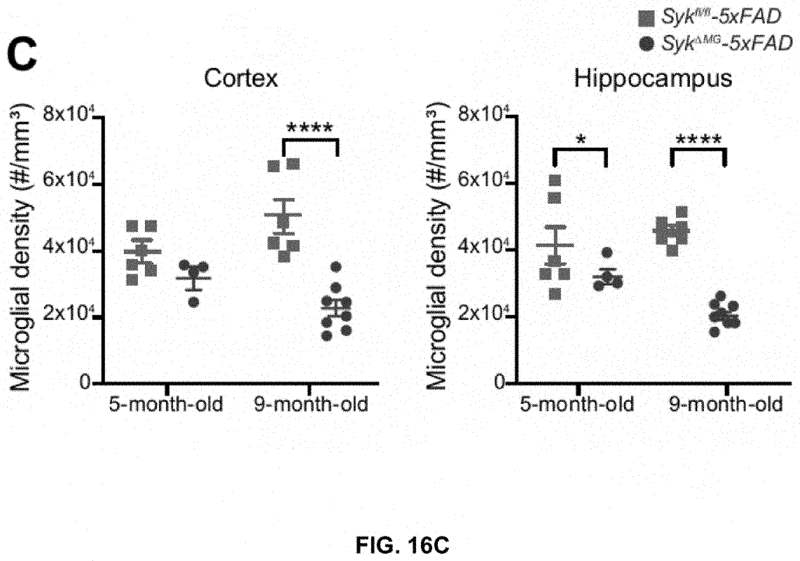

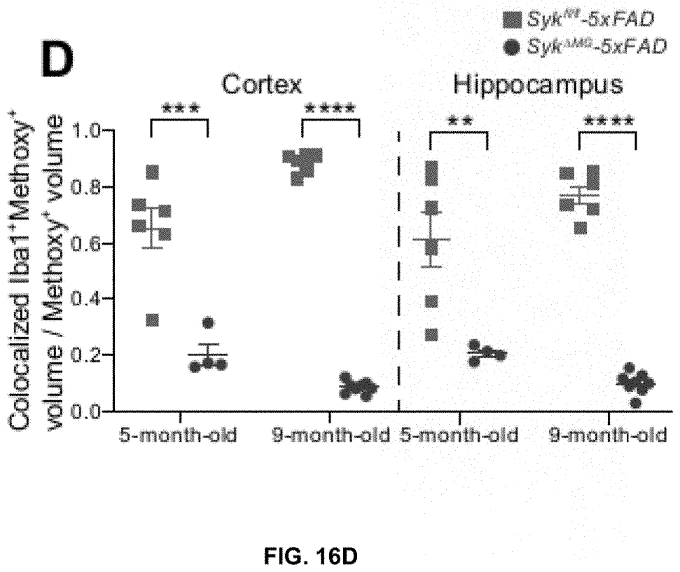

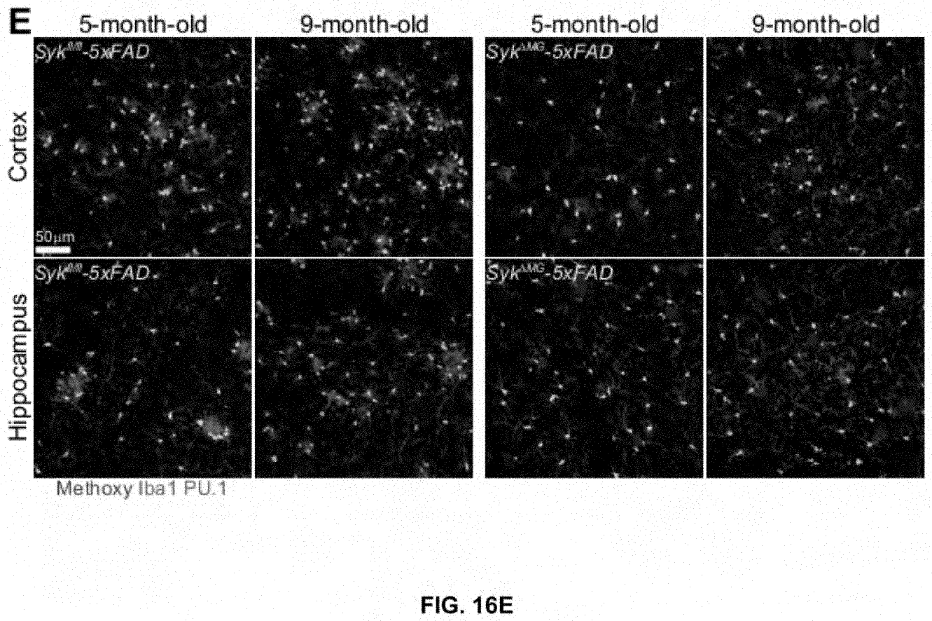

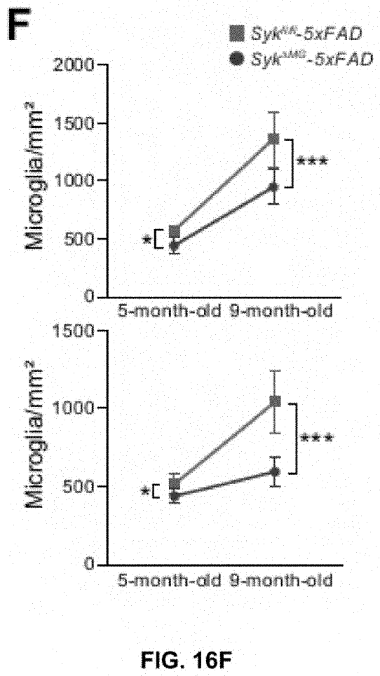

Our data showed that SYK signaling is required to elicit microglial responses to Aβ deposition. It was then asked whether SYK is also important to sustain microglia fitness at later stages of disease. To address this question, Syk^{ΔMG}-5xFAD mice and Syk^{fl/fl}-5xFAD control mice were fed with TAM-containing chow from 4 to 5 months of age (FIG. 16A), when mice have already started developing Aβ deposits and microgliosis. One cohort of mice was analyzed at the end of the TAM treatment (5 months of age), whereas a second cohort was returned to normal chow and analyzed at 9 months of age (FIG. 16A). At 5 months, slightly less pronounced density of microglia within a 15 μm radius of plaque borders was noted in the hippocampus, but not in the cortex of Syk^{ΔMG}-5xFAD mice. At 9 months of age, however, a significant reduction in the density of microglia within these areas surrounding plaques was observed in the cortex and hippocampus of Syk^{ΔMG}-5xFAD mice (FIG. 16B, FIG. 16C). Moreover, colocalized staining of Iba1+ methoxy-X04+ was significantly reduced in Syk^{ΔMG}-5xFAD mice in both the cortex and hippocampus at each time point (FIG. 16D). Next, it was determined whether the total number of microglia was also affected. Quantification of PU.1+ nuclei revealed that microglia numbers were slightly, but significantly, reduced in Syk^{ΔMG}-5xFAD mice as early as 5 months of age. At 9 months, microglia numbers were elevated in both groups; however, Syk^{ΔMG}-5xFAD mice had significantly fewer microglia than did Syk^{fl/fl}-5xFAD mice (FIG. 16E, FIG. 16F). Together, these results demonstrate that SYK is required for the maintenance of microglial clustering around Aβ plaques after the onset of disease.

SYK Deficiency Impairs Generation of DAM

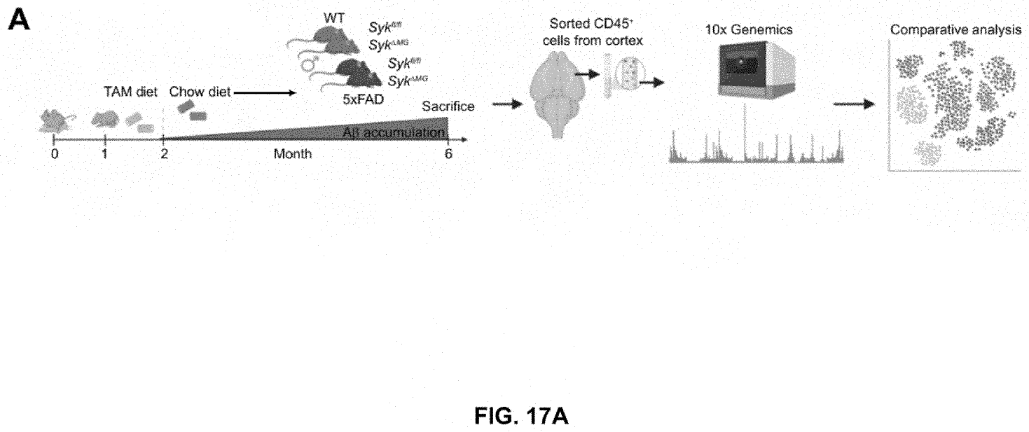

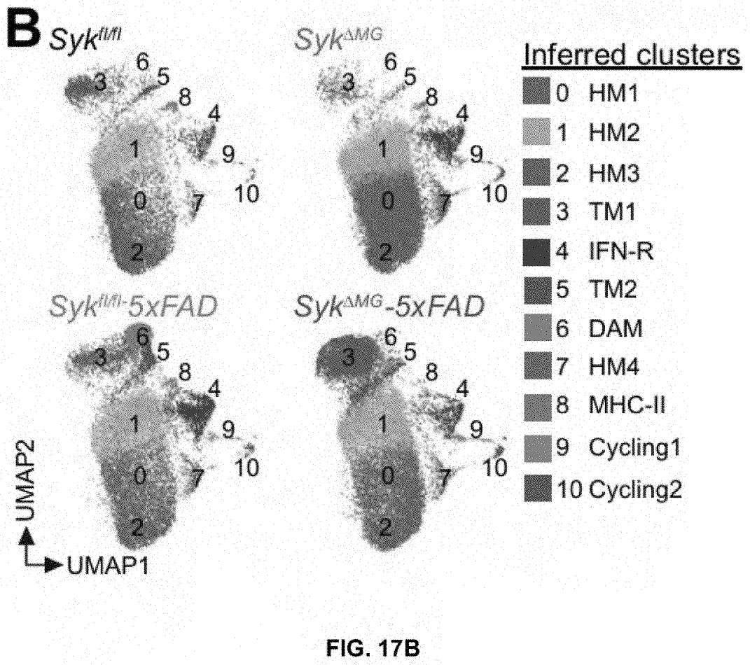

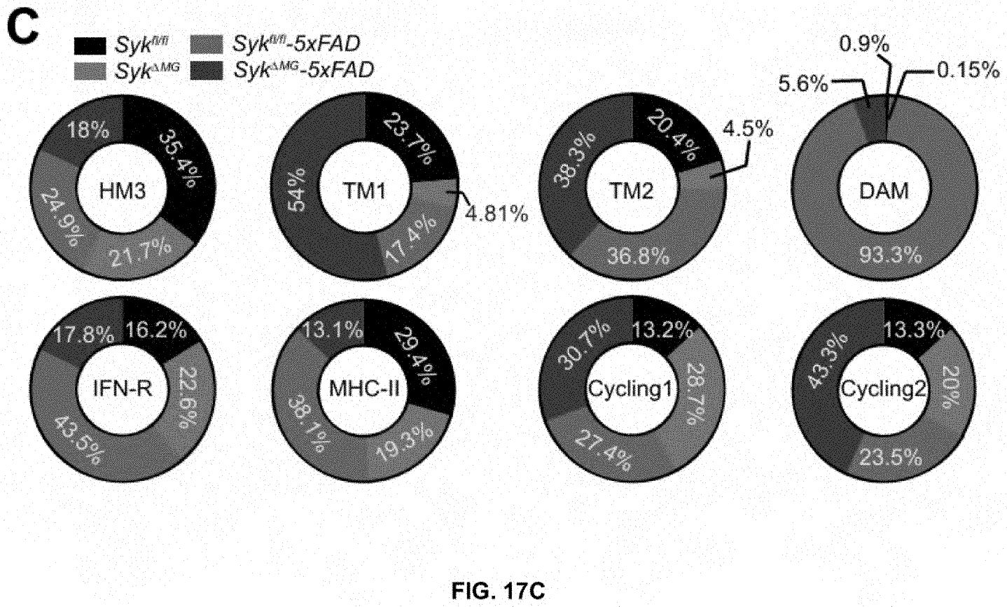

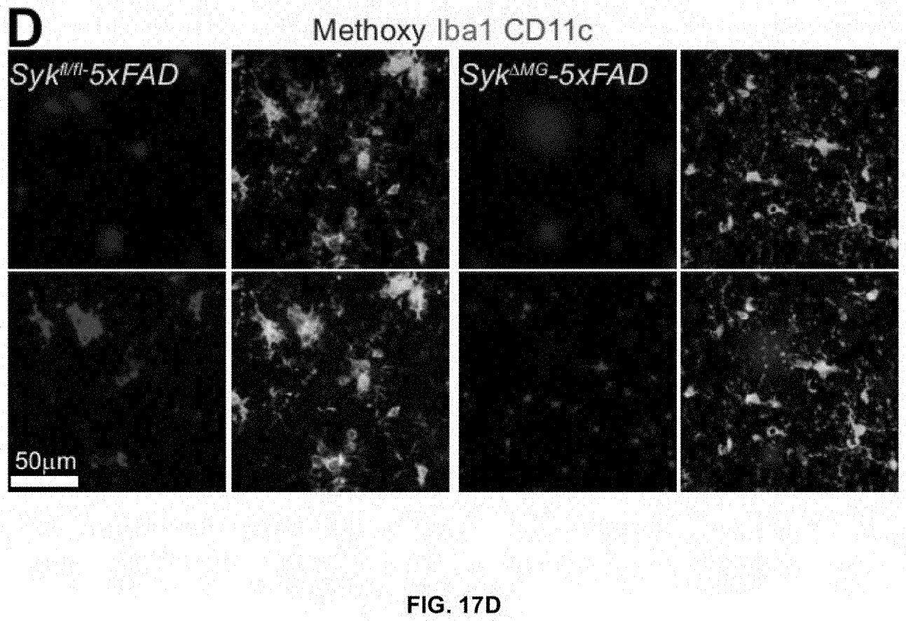

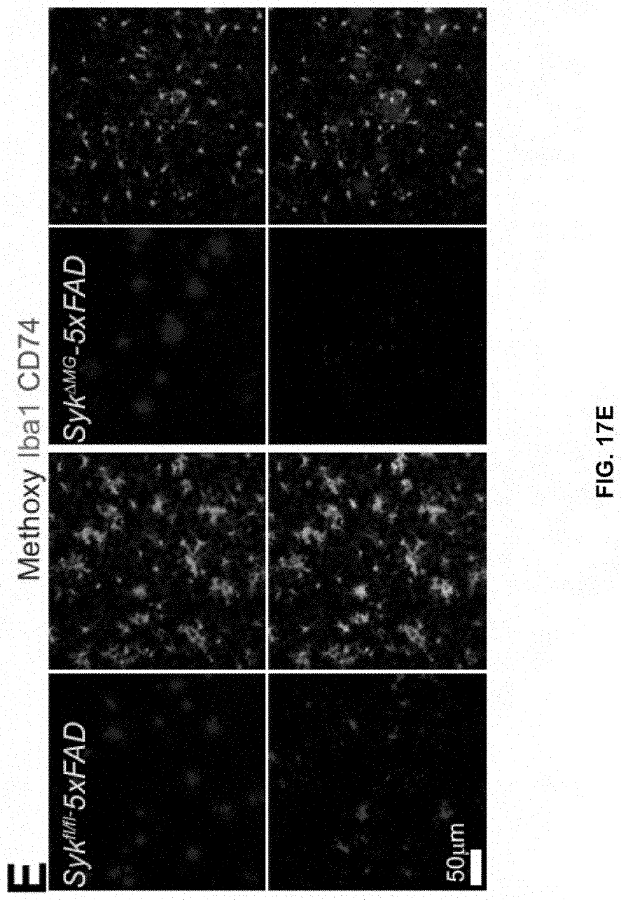

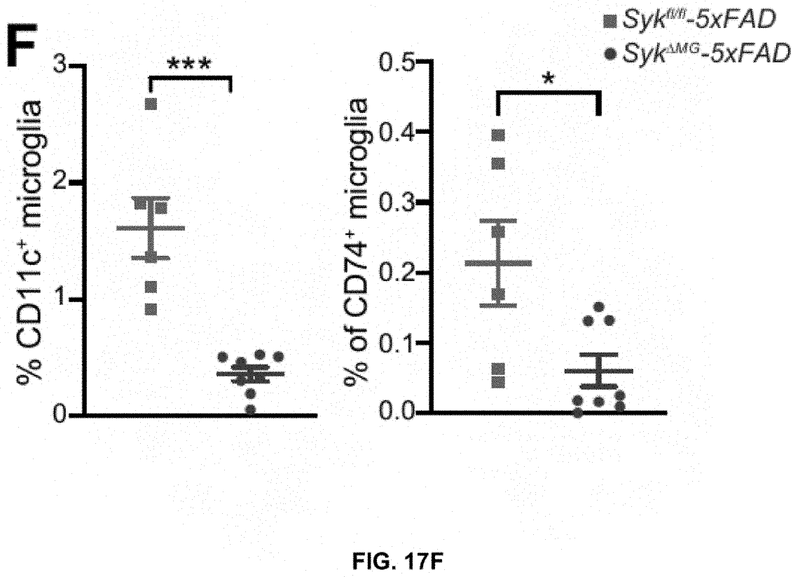

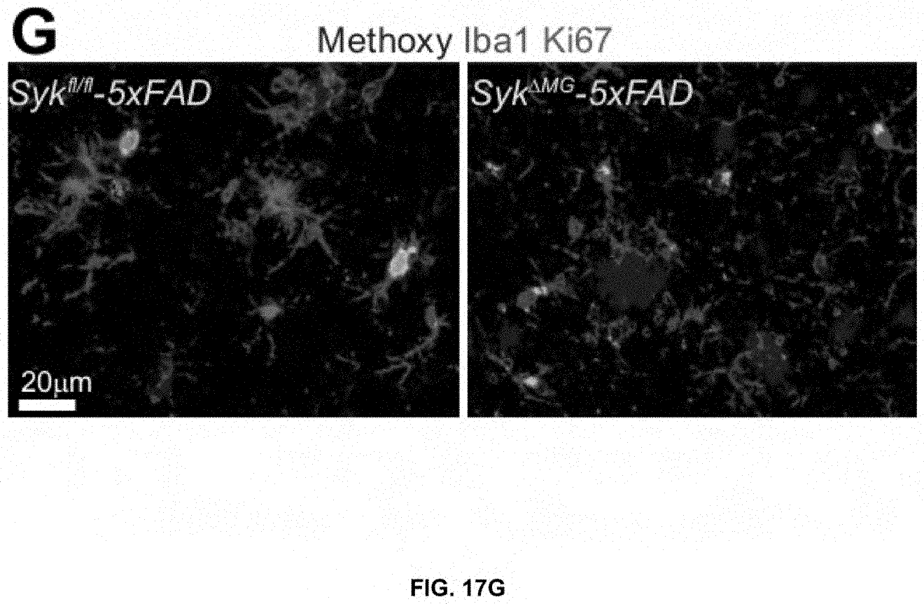

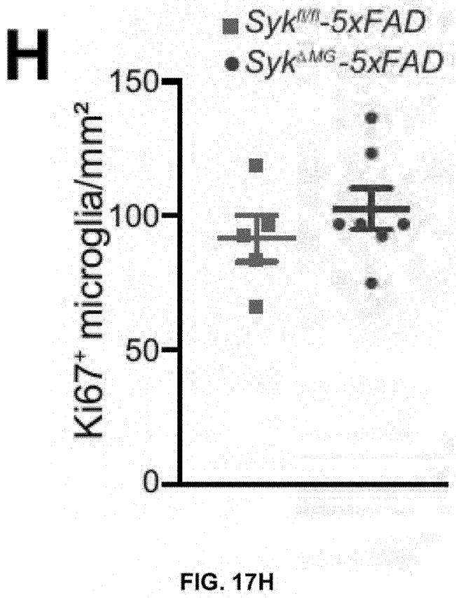

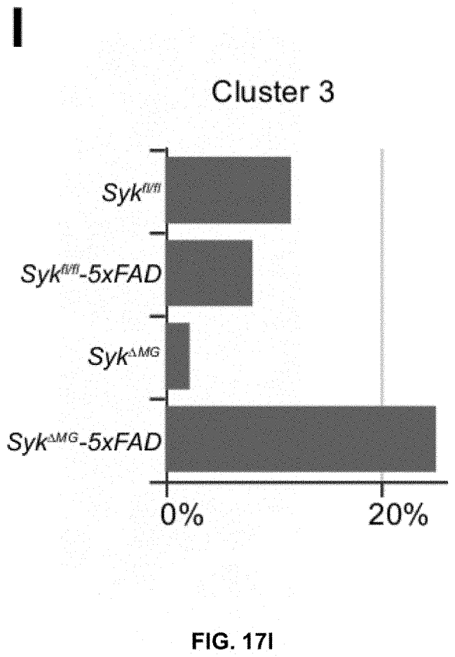

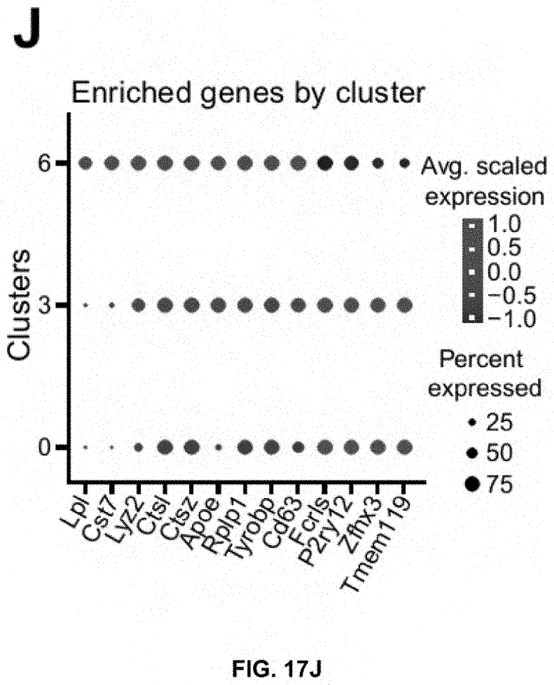

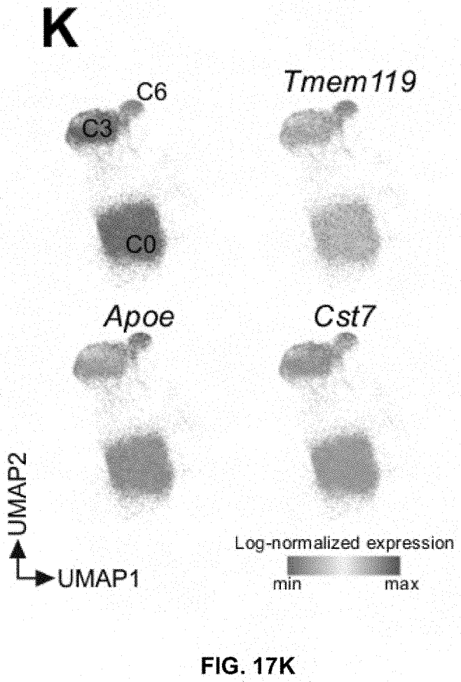

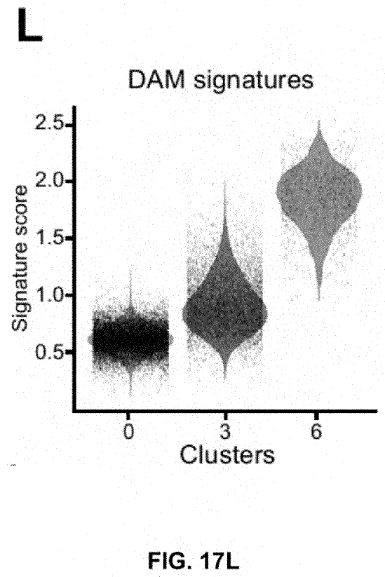

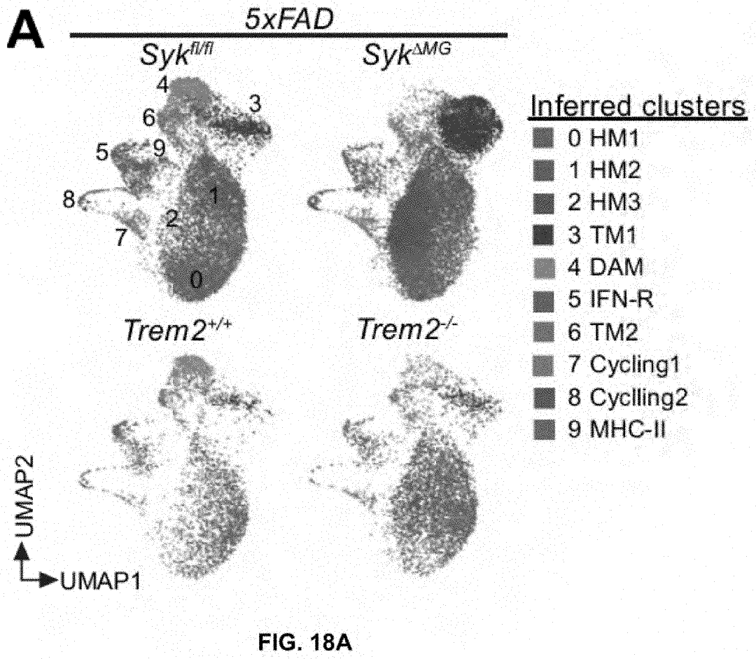

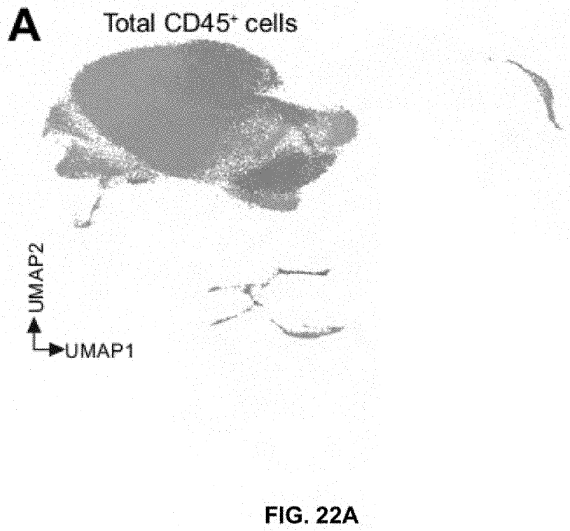

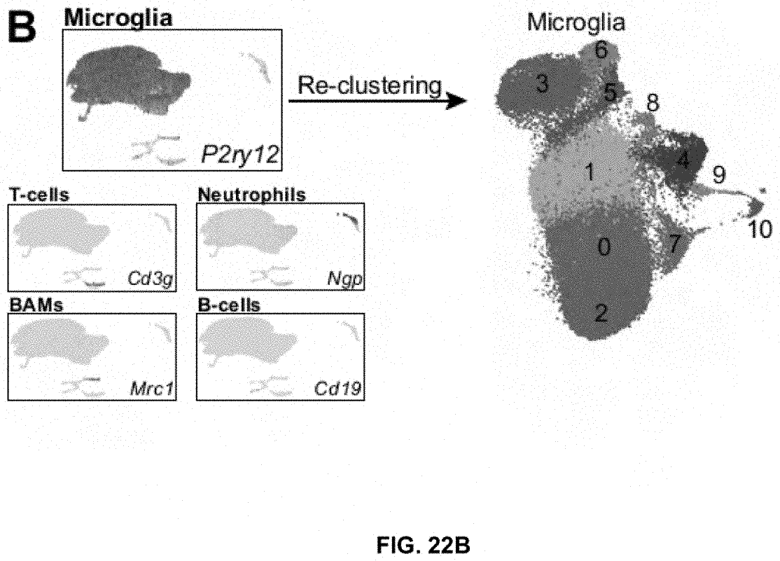

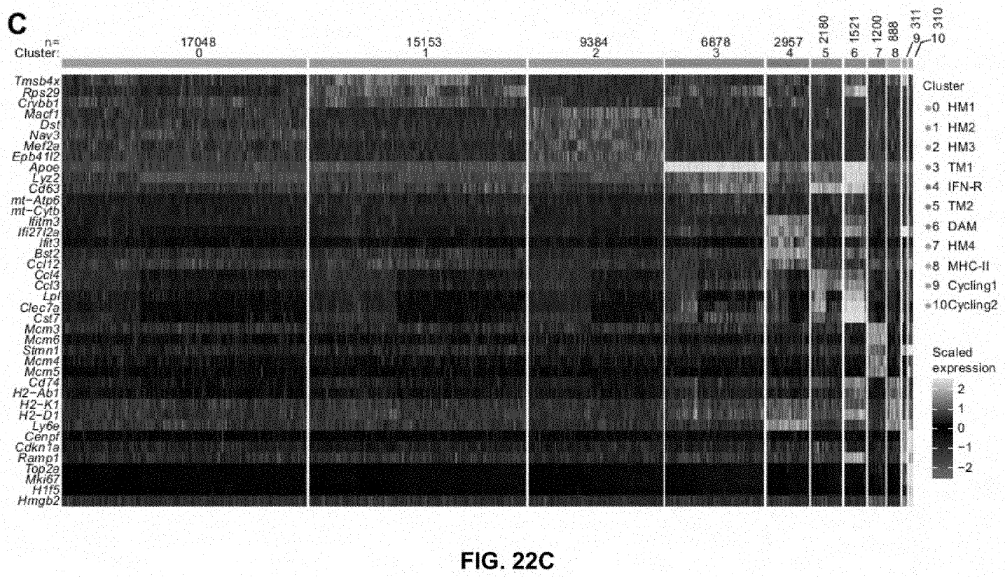

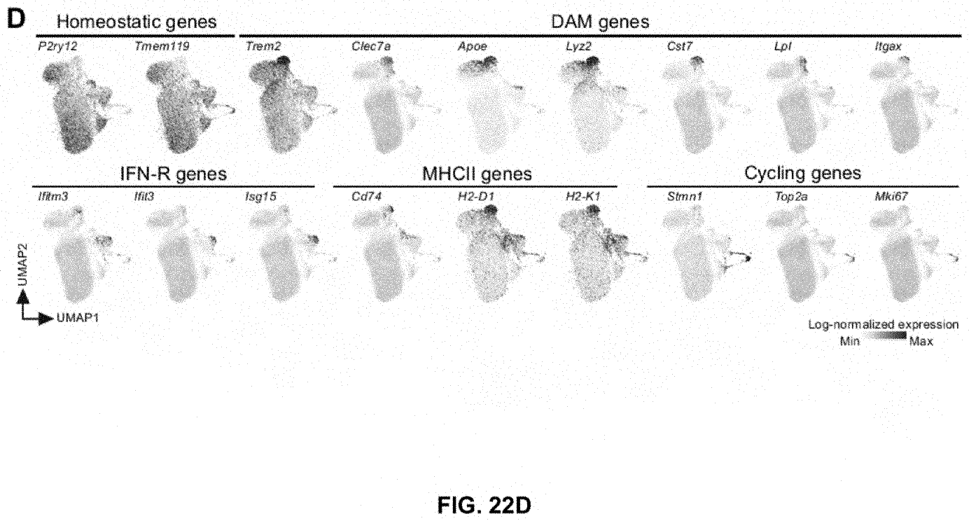

To characterize the impact of SYK deficiency on microglial transcriptome during amyloid pathology scRNA-seq was performed. Single/live CD45+ cells were sorted from Syk^{fl/fl}-5xFAD, Syk^{ΔMG}-5xFAD, Syk^{fl/fl}, Syk^{ΔMG} cortices (3 mice per group), and single-cell transcriptomes were generated using the 10x Genomics platform (FIG. 17A). After quality control, a total of 89,615 single cells were plotted on uniform manifold approximation projection (UMAP) dimensions for visualization. Unsupervised clustering revealed a total of 15 distinct clusters across all mice (FIG. 22A). These clusters were annotated based on differential expression of known cell type-specific marker genes. The preponderance of cells captured by the scRNA-seq dataset were microglia, with significantly fewer BAMs, T cells, B cells, and neutrophils (FIG. 22B). None of the genotypes had a significant impact on the clusters representing non-microglial cell types (data not shown).

Re-clustered microglia were next and removed subsets enriched for doublets, genes induced from tissue-dissociation and mitochondrial genes, resulting in 11 distinct clusters comprising 57,830 microglial cells (FIGS. 17B and 22C). Based on expression of published microglia subtype-specific marker genes. Homeostatic (clusters 0, 1, 2, and 7), DAM (cluster 6), MHCII (cluster 8), IFN-R (cluster 4), and proliferating (cluster 9, 10) microglia were identified (FIG. 17B, FIG. 22C, and FIG. 22D). Two clusters of microglia were also detected with intermediate signatures between homeostatic and DAM, which were defined as transitioning microglia (TM1 and TM2, clusters 3 and 5), which may represent prodromal stages of DAM. The representation of DAM, TM1, TM2, MHCII, IFN-R, and proliferating microglia clusters in each genotype was next examined (FIG. 17C). Notably, DAM appeared almost entirely represented in Syk^{fl/fl}-5xFAD mice (93.3%), with a minimal contribution from Syk^{ΔMG}-5xFAD mice (5.6%). MHCII and IFN-R microglia were abundant in Syk^{fl/fl}-5xFAD mice (38.1% and 43.5% respectively), but less represented in the other groups. All genotypes contributed similarly to cycling microglia, though Syk^{fl/fl} contributed less. Breakdown of TM1 among different genotypes showed that 54% of these cells were present in Syk^{ΔMG}-5xFAD microglia, while only smaller percentages were present in the other genotypes (FIG. 17C). Thus, SYK deficiency in microglia in the 5xFAD background mainly affected the generation of DAM. Interestingly, TM1 microglia (bona fide prodromal to DAM) were expanded in these mice. This suggests that, although a basal immune response to Aβ pathology is maintained in Syk^{ΔMG}-5xFAD mice, microglia are incapable to develop a fully developed DAM phenotype.

To validate these findings, immunofluorescence staining was performed (FIG. 17D and FIG. 17E) for CD11c (DAM marker gene) and CD74 (MHCII marker gene) (Syk^{ΔMG}-5xFAD). Consistent with transcriptomic data, a highly significant reduction of CD11c+ was found and, to a lesser extent, CD74+ microglia in Syk^{ΔMG}-5xFAD compared to Syk^{fl/fl}-5xFAD (FIG. 17F). Immunofluorescence staining for the proliferation marker Ki67 showed similar percentages of Ki67+ microglia in Syk^{ΔMG}-5xFAD and Syk^{fl/fl}-5xFAD mice (FIG. 17G, FIG. 17H), again supporting the scRNA-seq analysis. TM1 cells of cluster 3 were enriched in Syk^{ΔMG}-5xFAD mice for characterization (FIG. 17I). Analysis of genes differentially expressed among cluster 3, homeostatic cluster 0, and DAM cluster 6, revealed that cluster 3 expressed higher levels of microglia activation genes (Apoe, Ctsz, Ctsl, and Lyz2) than did homeostatic microglia. However, these genes were expressed at lower levels in cluster 3 than in DAM. Conversely, the homeostatic genes Tmem119, P2ry12, and Fcrls were more highly expressed in cluster 3 than in DAM but at lower levels than in homeostatic microglia. DAM also expressed high levels of Cst7 and Lpl which were not detected in either cluster 3 or cluster 0 (FIG. 17J, FIG. 17K). Analysis of the DAM signature among clusters 0, 3 and 6 indicated that cluster 3 occupies a transitional state between homeostatic microglia and DAM (FIG. 17L). It was concluded that SYK deficiency prevents the generation of a complete DAM phenotype in response to Aβ pathology, causing the accumulation of a microglial population prodromal to DAM, which expresses intermediate levels of both homeostatic and DAM genes, including Apoe.

SYK- and TREM2-Deficient Microglia Exhibit Distinct Defects of DAM Trajectories

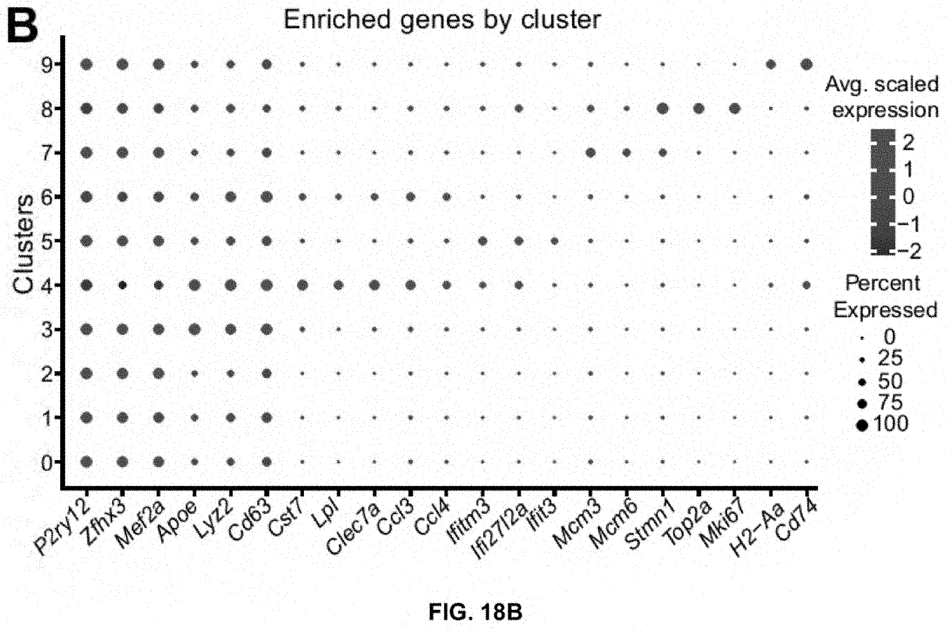

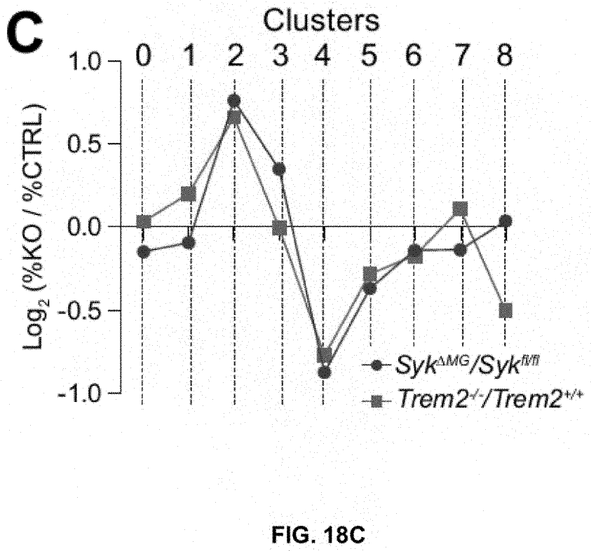

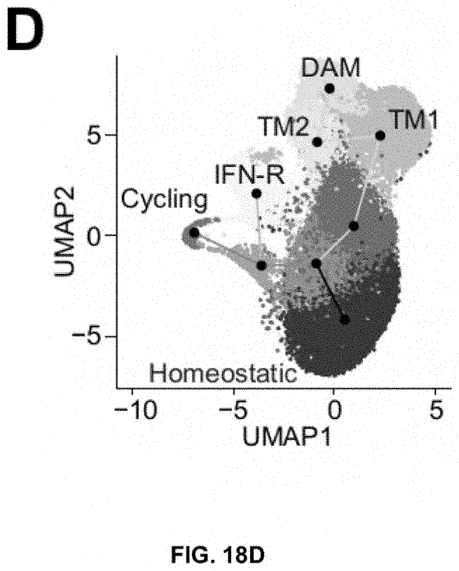

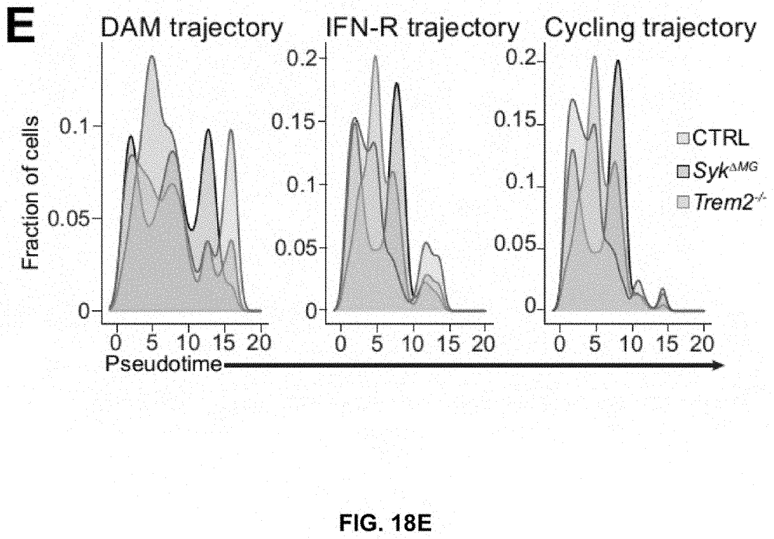

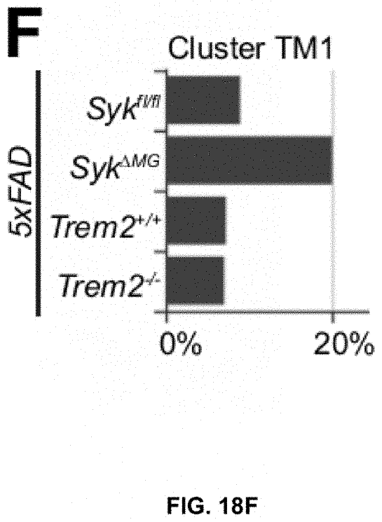

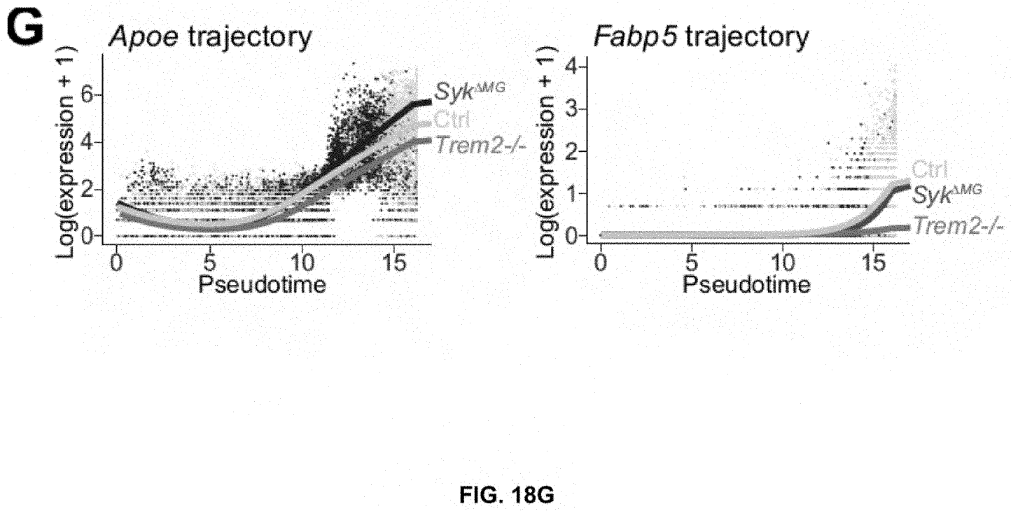

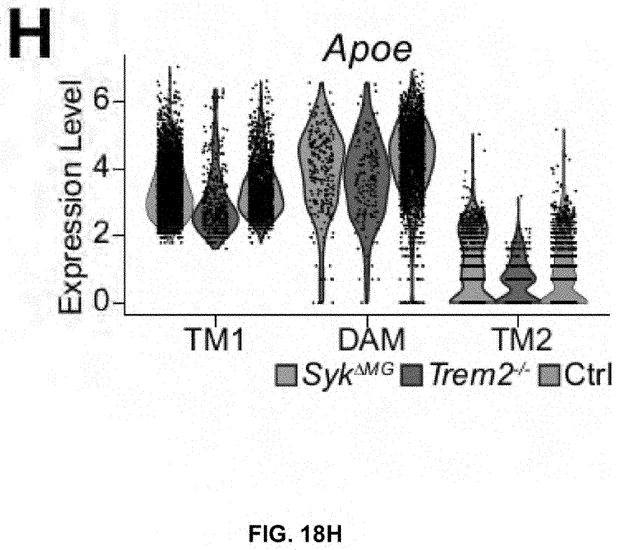

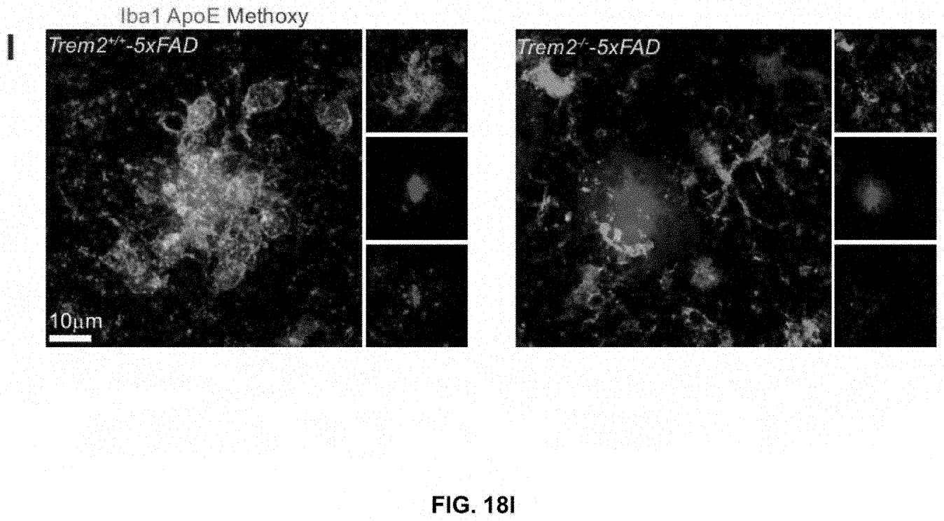

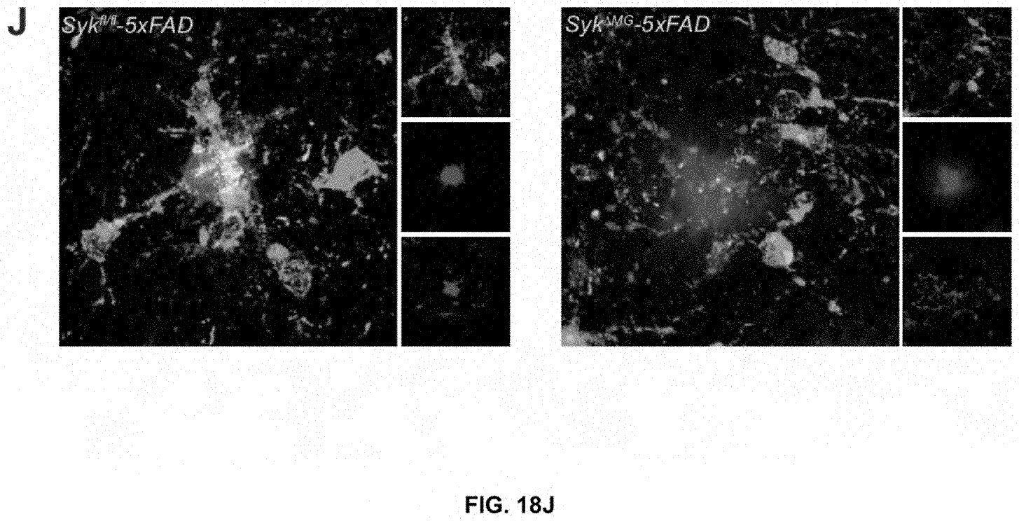

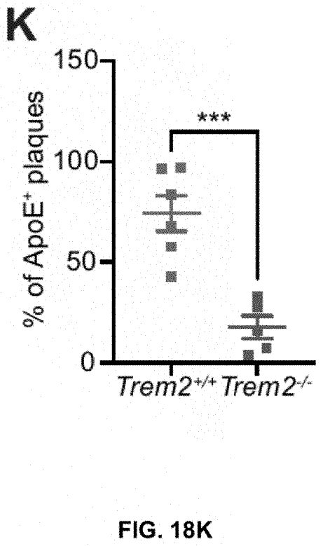

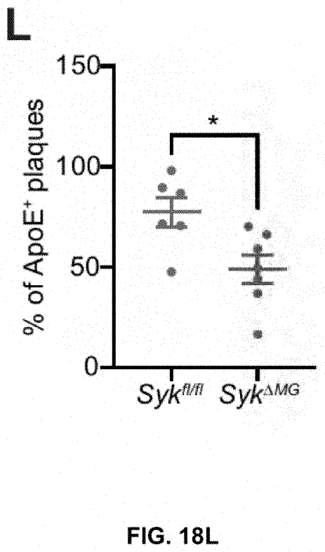

SYK is a critical component of TREM2 signaling, but it is also associated with other receptors, such as CLEC7A. Therefore, the SYK-deficiency impact on microglia activation was examined as compared with TREM2-deficiency. To do so, scRNA-seq data of Syk^{ΔMG}-5xFAD and Syk^{fl/fl}-5xFAD microglia was re-clustered together with newly generated scRNA-seq data of Trem2^{+/+}-5xFAD and Trem2^{-/-}-5xFAD microglia. Again, clusters were identified 10 recapitulating the same populations described above (FIG. 18A, 18B). Because the MHC-II cluster (cluster 9) contained only 167 cells, this population was excluded from further analyses. The representation of the remaining microglial clusters in each genotype was next analyzed. Compared to their respective controls, both Syk^{AmG} and Trem2^{-/-} microglia evinced a decrease in DAM (cluster 4), IFN-R (cluster 5), and TM2 (clusters 6, a DAM prodromal stage), whereas some homeostatic populations were expanded (clusters 1 and 2). However, an increase in the TM1 transitional stage (cluster 3) was evident in Syk^{AmG} microglia. A second discrepancy between the two genotypes was the abundance of cycling microglia (cluster 8), which was depleted Trem2^{-/-}, but not in Syk^{AmG} microglia (FIG. 18C). Pseudotime trajectory analysis corroborated that the homeostatic population differentiated into three terminal stages (DAM, IFN-R and cycling microglia). Importantly, TM1 and TM2 stages appeared to precede DAM, but not IFN-R and cycling clusters (FIG. 18D). The distribution of Trem2^{-/-} and Syk^{AmG} microglia was next considered along these three trajectories. Trem2^{/-} microglia were associated with a complete blockade of all fates. By contrast, Syk^{AmG} microglia were partially blocked along the DAM trajectory with an arrest at the transitional stage TM1, the IFN-R trajectory was moderately impaired, and no defect was observed in the cycling trajectory (FIG. 18E). These data indicate that, unlike Trem2 microglia, Syk^{AmG} microglia can initiate a DAM differentiation program, but are unable to reach a DAM terminal stage, resulting in a progressive accumulation of the prodromal stage TM1 (FIG. 18F).

Differentially expressed genes (DEGs) between these two genotypes along the DAM trajectory were then identified. 16 DEGs were identified, including the DAM signature genes Apoe and Fabp5, which were both reduced in Trem2^{-/-} microglia compared to controls. (FIG. 18G). Focusing specifically on the TM1 cluster, Apoe was sharply enriched in Syk^{ΔMG} microglia, compared to Trem2^{-/-} microglia (FIG. 18H). Collectively, these data suggest that induction of Apoe is TREM2-dependent but SYK-independent, at least in part. Yet, absence of SYK impedes the acquisition of a complete DAM signature. To further corroborate this conclusion, immunofluorescence staining was performed for ApoE on brain sections of Trem2^{+/+}-5xFAD, Trem2^{-/-}-5xFAD, Syk^{f′/f′}-5xFAD and Syk^{ΔMG}-5xFAD mice (FIG. 18I, 18J). Both Trem2^{-/-}-5xFAD and Syk^{ΔMG}-5xFAD mice had a reduced percentage of ApoE⁺ plaques compared to respective controls, which is consistent with the significant reduction of DAMs in both genotypes. However, a more significant reduction in ApoE⁺ plaques was evident in Trem2^{-/-}-5xFAD mice than in Syk^{ΔMG}-5xFAD mice (−76.2% vs −36.7% respectively) (FIG. 18K, 18L). Moreover, the staining pattern of plaque-associated ApoE in Syk^{ΔMG}-5xFAD mice appeared more diffuse than that seen in Sy^{f′/f′}-5xFAD mice. This may be due to the defective clustering of Syk^{ΔMG} microglia and/or tempered ApoE production. Altogether, these data suggest that Aβ pathology may elicit a TREM2-dependent, yet SYK-independent, pathway in microglia that supports the expansion of a prodromal DAM stage simultaneously expressing homeostatic genes and Apoe.

Anti-CLEC7A Improves Microglia Activation in 5xFAD Mice Carrying a TREM2 Hypofunctional Variant

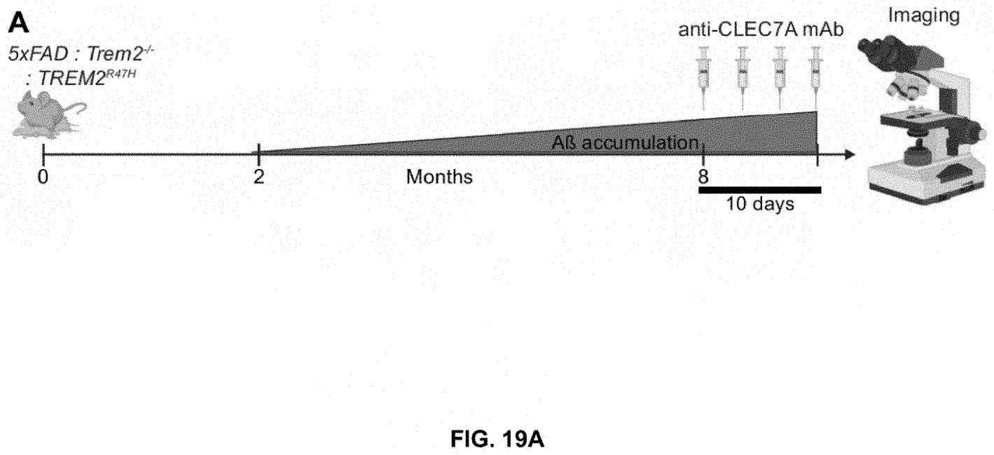

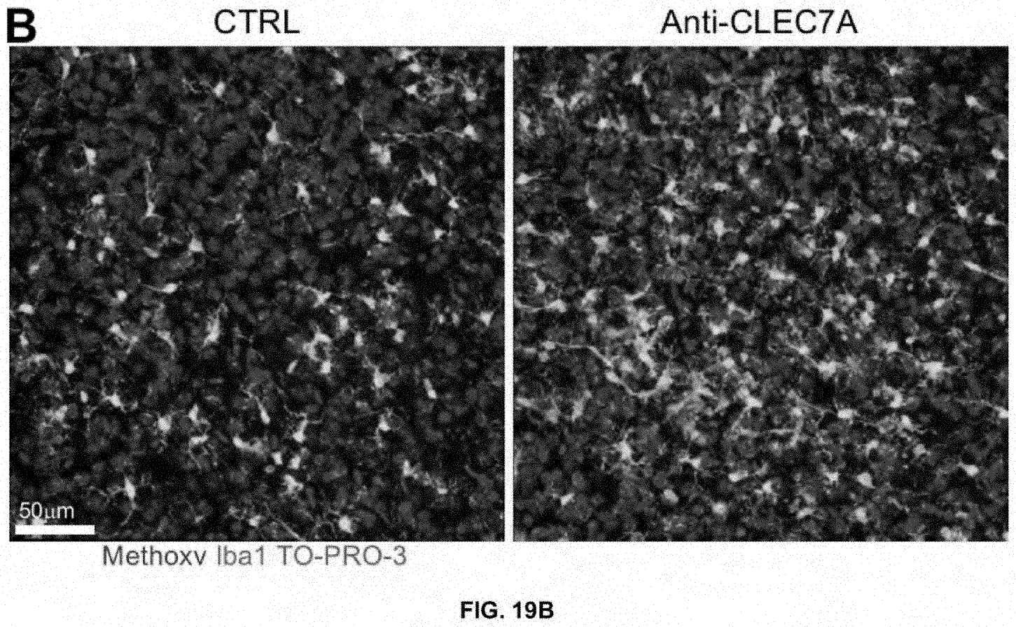

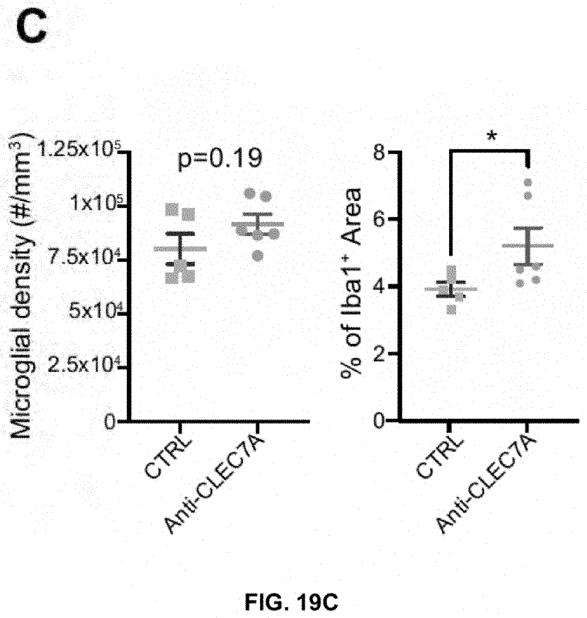

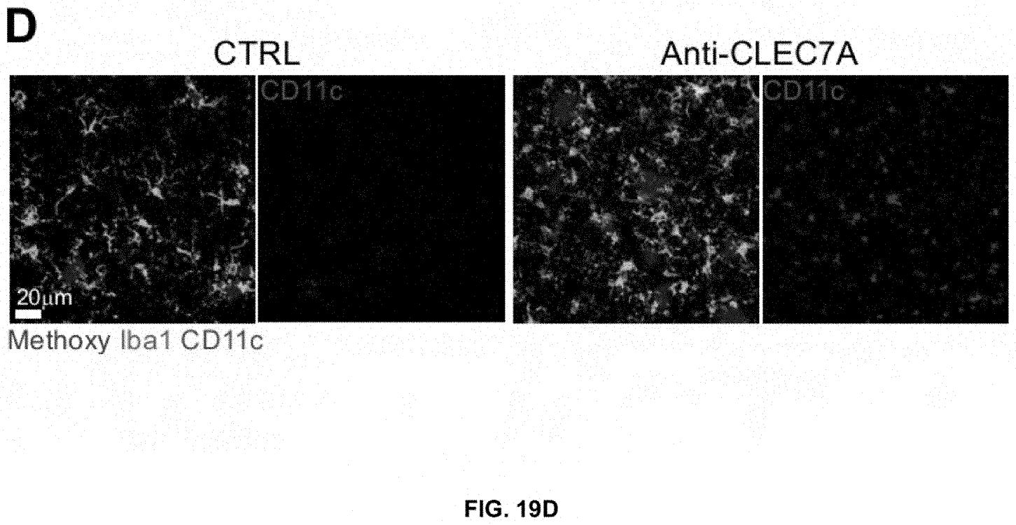

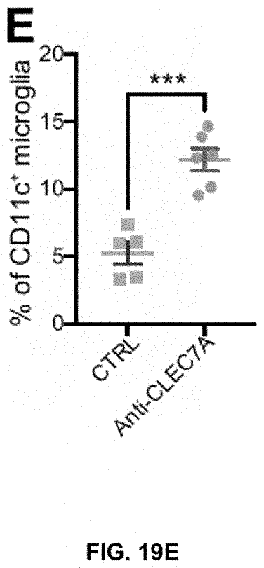

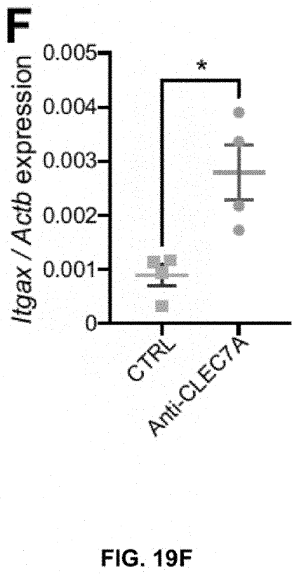

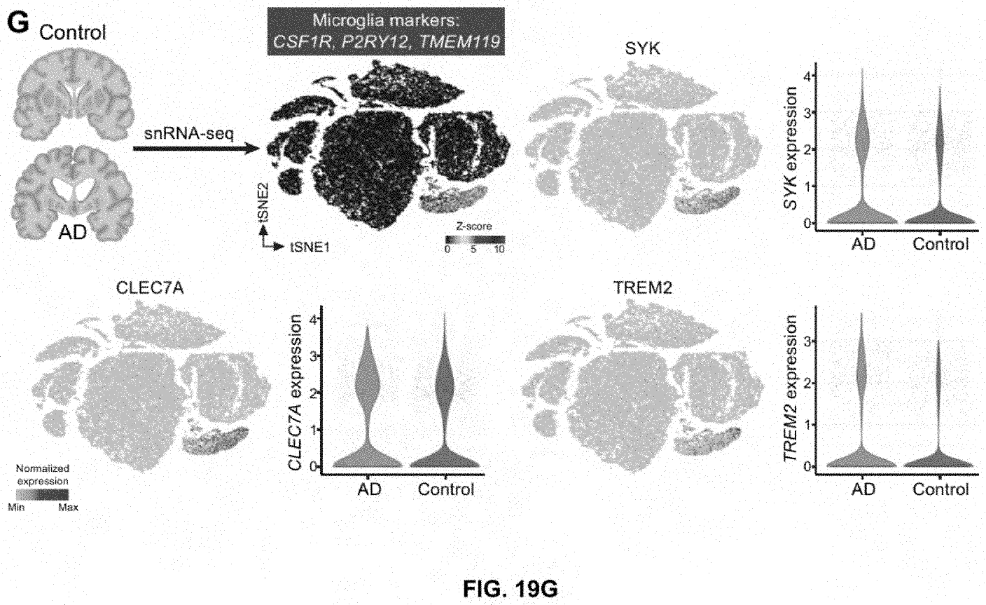

5xFAD mice carrying the human R47H hypofunctional variant of TREM2 (TREM2^{R47H}) associated with AD have a blunted microglia response to Aβ pathology in comparison to 5xFAD mice carrying the common variant of TREM2 (TREM2^{c})). Since the TREM2^{R47H} variant is unable to activate SYK, it was explored whether SYK activation could be rescued in this mouse model by engaging an alternative surface receptor that recruits SYK. CLEC7A was chosen (alias DECTIN1), a member of the C-type lectin receptor family that is expressed in neutrophils, alveolar and peritoneal macrophages, and dendritic cells, and is upregulated in microglia during neurodegeneration. CLEC7A recognizes β-glucan, a component of the fungal wall, promoting antifungal responses in macrophages. The CLEC7A cytoplasmic domain contains a hemi-ITAM domain that serves as a docking site for the dual SH2 domains of SYK, leading to direct recruitment and activation of SYK. In turn, SYK elicits macrophage activation and enhances TLR and NOD2 signaling, but it is dispensable for phagocytosis. To engage CLEC7A in microglia during Aβ pathology, TREM2^{R47H}-5xFAD mice i.p. were injected with 60 mg/kg of agonistic anti-CLEC7A monoclonal antibody 2A11 every three days for 10 days. The Fc portion of the antibody was mutated to prevent binding to Fc receptors on microglia. Twenty-four hours after the final injection, mice were analyzed by imaging (FIG. 19A). A trend towards increased density of plaque-associated microglia and a significant increase in the overall Iba1 staining was evident in mice that received the anti-CLEC7A treatment (FIG. 19B, 19C). Additionally, microglia from the treatment group upregulated expression of the DAM marker CD11c (FIG. 19D, 19E), indicating increased activation. Increased expression of Itgax (encoding CD11c) was validated by RT-PCR in brain lysates (FIG. 19F). Based on these data, CLEC7A engagement in microglia with hypofunctional TREM2 can, at least partially, rescue microglial responses to Aβ. Lastly, published snRNA-seq data of human AD and age matched controls was mined for expression of SYK and CLEC7A mRNA. TREM2 expression was used for comparison. As described and confirmed herein, SYK and CLEC7A are highly expressed in human microglia in both AD and control subjects (FIG. 19G). Thus, the CLEC7A-SYK pathway is a potential therapeutic target for microglia activation in AD.

Discussion

The present disclosure demonstrates that SYK signaling is a central node in microglia responses to Aβ pathology. Conditional deletion of SYK in microglia prior to disease onset dramatically impaired the ability of microglia to encase Aβ plaques, resulting in augmented Aβ deposition, increased neuronal damage, and worsened behavioral deficits. When SYK deletion was induced at later stages, microglial clustering around Aβ plaques was reversed, suggesting that SYK is required not only for generation but also for maintenance of microglia responses to Aβ. Mechanistically, SYK deficiency impaired the activation of the PI3K-AKT-mTOR axis, which provides energy and substrates for anabolic processes that sustain microglia activation. In parallel, SYK deficient microglia escalated autophagy, which may provide an alternative catabolic pathway to compensate for defective mTOR signaling. Autophagy may partially delay microglia dysfunction and Aβ pathology, although it may not be sufficient to sustain microglia responses to Aβ plaques in the long term. Using scRNA-seq, intermediate and terminal stages of microglial response to Aβ were resolved, showing that SYK-deficient microglia fail to acquire DAM, and to a lesser extent, MHCII and IFN-R activation profiles. Importantly, As demonstrated herein, systemic administration of an agonistic anti-CLEC7A antibody to 5xFAD mice carrying the hypofunctional TREM2^{R47H} variant promotes SYK signaling and improved microglial responses to Aβ plaques. Since both CLEC7A and SYK are expressed in human microglia, CLEC7A emerges a potential therapeutic target to boost microglia activation in AD.

An unexpected result of the present disclosure is that scRNAseq data from TREM2-deficient and SYK-deficient microglia do not entirely overlap. While both SYK and TREM2 deficiencies did impede the generation of DAM, SYK deficiency led to the expansion of a microglia cluster simultaneously expressing DAM genes (Apoe and Cathepsins), and homeostatic genes (Tmem119 and P2ry12). Lineage trajectory analysis defined this cluster as a stage prodromal to DAM. This microglia population was much less represented in TREM2-deficient mice, indicating a SYK-independent but TREM2-dependent origin. Moreover, TREM2 deficiency impaired the generation of all microglia terminal stages, namely DAM, MHCII, IFN-R, and proliferation clusters. By contrast, SYK deficiency primarily impaired DAM formation, but left the proliferation cluster unaffected. One possible explanation for these discrepancies is that TREM2 may transmit some intracellular signals through a SYK-independent pathway. A possible candidate may be the adapter protein DAP10, which has been shown to associate with TREM2 and directly recruit PI3K. This pathway may promote the differentiation of a prodromal stage of DAM, induce ApoE expression and facilitate microglia proliferation. Since ApoE is a ligand of TREM2, this pathway may generate a positive feedback loop sustaining this population. Notwithstanding, given the profound defect of microglia clustering observed in Syk^{ΔMG}-5xFAD mice, the SYK signaling pathway seems irreplaceable for full DAM differentiation and effective containment of Aβ pathology. Accordingly, the present disclosure advances the mechanistic insights into the transition from homeostatic microglia to DAM, improving the understanding of microglial response to Aβ pathology.

Microglia, like other tissue macrophages, express a large network of germline-encoded activating receptors that transmit signals through ITAMs. Many of these innate immune receptors may contribute to shape the magnitude and duration of microglia responses to protein aggregates, dead neurons, myelin debris and other molecular patterns associated with AD. By activating SYK, these receptors sustain the energetic and anabolic pathways required for continued activation. As disclosed herein, engagement of CLEC7A was able to offset the defect of microglia activation in TREM2^{R47H} mice in vivo. Beyond CLEC7A, the present disclosure demonstrates that therapeutic activation of microglia in AD may be achieved using agonists for the broad range of microglia receptors that activate ITAM pathways converging on SYK.

Methods

Experimental Model and Subject Details

Mice. Mice were of mixed sexes. Mice within experiments were age and sex matched. Syk^{fl/fl}- and Syk^{DMG}-5xFAD mice were generated by crossing the Syk^{fl/fl}-Cx3crl^{CreERT2/+} mice with Syk^{fl/fl}-5xFAD. Trem2^{R47H}-5xFAD mice were previously described elsewhere. For peritoneal macrophage isolation 6-12 weeks old mice were used. Mice used herein were sacrificed at indicated time points. All animal experiments were conducted in compliance with Institutional regulations, under authorized protocols #19-0981 approved by the Institutional Animal Care and Use Committee.

Primary cells. Peritoneal macrophages were isolated from sex and age matched mice. To prepare peritoneal macrophages, Sykfl/fl and Syk^{fl/fl}-Cx3crl^{CreERT2/+} mice were i.p. injected with tamoxifen (dissolved in corn oil at final concentration of 20 mg/mL) at 75 mg/kg body weight for 4 consecutive days followed by thioglycolate injection i.p. After 3 days, peritoneal macrophages were harvested by with an 18-gauge needle in 10-15 ml of PBS. Cells were counted and plated at 6-well plates in RPMI supplemented with Glutamax, penicillin/streptomycin, non-essential amino acids, pyruvate, and 10% heat inactivated fetal bovine serum (complete RPMI) for other 2 days before use.

Method Details

Mice. For generation of Syk^{fl/fl}- and Syk^{DMG}-5xFAD mice 1- or 4-month-old littermates were fed a tamoxifen diet for 4 weeks followed by replacement with regular chow. Treated mice were sacrificed at 6-, 9- or 5-, 9-months of age as indicated in the RESULTS. For anti-CLEC7A antibody treatment 8-month-old Trem2^{R47H}-5xFAD mice received intraperitoneal injection of anti-CLEC7A antibody (2A11), or anti-human ILT1-Fc mutated (clone 135.5) antibody as a control, for 4 times within 10 days. Mice were sacrificed within 24 h after the last injection for assessment.

Flow cytometry. Cells were incubated with Fc block prior to staining. The following antibodies were used: CD45-PE or -APCCy7 (clone 30-F11), CD11b-APC (clone M1/70), Ly6C-APCCy7 (clone HK1.4), Ly6G-FITC (clone 1A8); P2RY12-BV421 (clone S16007D), Syk-PE (clone 5F5). Cells, stained with indicated antibodies for 30 min on ice, were acquired (LSR FORTESSA) and analyzed using FlowJo software. Cell viability was determined by Aqua LIVE/Dead-405 nm staining (Invitrogen), and negative cells were considered viable. Foxp3/Transcription Factor Staining Buffer Set (eBioscience) was used for intracellular staining.

Cell sorting. Cells from brain were isolated from the indicated animals. CD45loCD11b+ microglia or CD45+ cells in the brain were fluorescence-activated cell-sorted (FACS Aria II, BD Bioscience) directly into 2% FBS in PBS for TEM or immunoblot lysates or 0.04% BSA for single-cell RNAseq.

Immunoblotting. Sorted microglia or thioglycolate elicited peritoneal macrophages were lysed in Cell Signaling cell lysis buffer for 20-30 min on ice. Cell extract was centrifuged at 12000 rpm for 15 min and supernatant was collected and subjected to protein estimation. Equal amount of protein from each sample mixed in 4× Laemmli sample buffer was loaded on 4-20% mini protein TGX gels from Bio-Rad and run using Tris-Glycine-SDS buffer. Resolved proteins were transferred on PVDF membranes. Membranes were washed using TBST (Tris-buffered-saline with 0.1% tween 20), blocked using 5% BSA or non-fat dried milk and incubated in primary antibody overnight at 4° C. The following primary antibodies were used: anti-Syk (1:1000, Rabbit monoclonal, Cell Signaling Technology), anti-phospho Akt 473 (1:1000, rabbit monoclonal, Cell Signaling Technology), anti-LC3 (1:1000, rabbit polyclonal, Cell Signaling Technology), anti-phospho S6K (1:1000, rabbit monoclonal, Cell Signaling Technology), anti-phospho NDRG1 (1:1000, rabbit polyclonal, Cell Signaling Technologies), anti-beta-actin-HRP (1:4,000, mouse monoclonal, Santa Cruz Biotechnology). After incubation in primary antibody, membranes were washed and incubated in HRP conjugated secondary antibody for 1 h at room temperature (RT), washed again and developed using SuperSignal West Pico Chemiluminescent substrate inside Bio-Rad ChemiDoc MP imaging system.

Sample preparation for TEM. Sorted microglia were fixed in 2% paraformaldehyde (PFA)/2.5% glutaraldehyde (Ted Pella Inc.) in 100 mM sodium cacodylate buffer pH 7.2 for 1 h at RT. Following washes with sodium cacodylate buffer, cells were embedded in 2.5% agarose and post-fixed with 1% osmium tetroxide (Ted Pella Inc.) for 1 h. After three washes in dH2O, samples were en bloc stained in 1% aqueous uranyl acetate (Electron Microscopy Sciences) for 1 h. Samples were washed with dH2O, dehydrated in an ethanol series, and infiltrated with Eponate 12 resin (Ted Pella Inc.). Ultrathin sections (95 nm) were cut with a diamond knife using a Leica Ultracut UCT ultramicrotome (Leica Microsystems Inc.) and counterstained with uranyl acetate and lead citrate. Samples were analyzed and imaged on a JEOL 1200 EX II transmission electron microscope (JEOL USA Inc.) equipped with an AMT 8-megapixel digital camera and AMT Image Capture Engine V602 software (Advanced Microscopy Techniques). Quantitative evaluation was performed by taking random images of 30 cells of comparable size and sectioned through the nucleus (indicating center of cell) and measuring the cross-sectional area using ImageJ (National Institutes of Health). Data is expressed as the 1) total number of a multivesicular/multilamellar structures per cross-sectional area of cytosol and 2) the total cross-sectional area of multivesicular/multilamellar structures per area of cytosol.

Immunofluorescence staining of brain samples. Briefly, confocal microscopy analysis was performed after transcardial perfusion with ice-cold PBS containing 1 U/ml of heparin, brains were fixed in 4% PFA overnight at 4° C. followed by incubating in 30% sucrose overnight at 4° C. before embedding in a 2:1 mixture of 30% sucrose and optimal cutting temperature compound. 40-μm serial coronal sections were cut on a cryo-sliding microtome. Sections were blocked with PB S+5% bovine serum albumin and permeabilized with 0.5% Triton-X 100 in blocking solution. Primary antibodies were added at indicated different combination overnight at a dilution of 1:1,000 (rabbit monoclonal, Cell Signaling Technology) or 1:500 (goat polyclonal, Abcam) for Iba1, 1:1,000 for Pu.1 (rabbit monoclonal Cell Signaling Technology), 1:500 for AJ342 (rabbit recombinant monoclonal, Thermo Fisher Scientific) or 1:1,000 for AJ31-16 (6E10, BioLegend), 1:1,000 for Lamp1 (Rat Monoclonal, BioLegend), 1:500 for LC3-Alexa Fluor® 488 (rabbit monoclonal, Cell Signaling Technology), 1:1,000 for CD11c (rabbit monoclonal, Cell Signaling Technology), 1:300 for CD74-Alexa Fluor® 647 (Rat monoclonal, BioLegend), 1:500 for Ki67 (Rabbit polyclonal, Abcam), 1:300 for ApoE (biotinylated HJ6.3). Secondary antibodies were added as follows: anti-goat IgG Alexa-Fluor®488 1:1,000 (donkey polyclonal, Abcam), anti-rabbit IgG Alexa Fluor® 555 1:1,000 (donkey polyclonal, Abcam), anti-goat IgG Alexa Fluor® 647 1:1,000 (donkey polyclonal, Abcam), anti-rabbit IgG Alexa Fluor® 647 1:1,000 (goat recombinant polyclonal, Invitrogen), anti-rat IgG Alexa Fluor®647 (chicken polyclonal, Invitrogen), Streptavidin-Alexa Fluor® 647 1:500 (BioLegend) for 1.5 h at RT. TO-PRO-3 iodide (300 nM; Thermo Fisher Scientific) was used to counterstain for nuclei, and Aβ plaques were labeled with methoxy-X04 (3 μg/ml; Tocris). The respective areas were captured using a conventional fluorescence microscope (Nikon Eclipse 80i microscope) or a confocal microscope (Nikon A1Rsi+). Images were then processed with Imaris 7.7 (Bitplane).

Microglia clustering analysis. The density of microglial clustering around AJ3 plaques within z stacks were determined by using the Spots and Surface function of Imaris followed by running an automated scripts in MATLAB as described before. Briefly, the Spots function was used to identify microglia within the Iba1 and TO-PRO-3 colocalized channel and the volume as well as location of AJ3 plaques was reported by Surface function on methoxy-X04 image data. The density of microglia within 15-μm of plaque surfaces was estimated in MATLAB mathematically. The volumes of dystrophic neurites around plaques were calculated by a similar approach in which the Surfaces function was used to identify the volumes and location of methoxy-X04 as well as Lamp1 positive signals within z stacks. Microglial coverage around AJ3 plaques was calculated as the total Iba1⁺ methoxy-X04⁺ volume divided by the total methoxy-X04⁺ volume. Iba-1 intensity profile was generated by Surface function and the number of microglia was determined by the Spots function of Imaris on Pu.1 image data.

Quantification of A_{J3} plaques pathology. The measurement of total methoxy-X04 and AJ342 area coverage was performed, briefly, with cortex regions or a similar region encompassing portions of cortex drawn in ImageJ (National Institutes of Health) from individual image and a threshold in which a fixed intensity was much greater than the mean for the selected region was used for calculating the percentage of area coverage automatically by batch processing in ImageJ. Two or three brain sections per mouse were used for quantification. The plaque load for each mouse was shown as the average of two or three sections. Quantification of Iba1 coverage in the cortex was performed by a similar procedure, with different threshold settings. The conformation of individual plaque in each condition was determined. The proportion of distinct plaque morphology was calculated by normalizing the total number of plaques from each z-stack image.

Quantification of microglial activation. For measurement of CD11c+ pixels within Iba1+ pixels, the Spots function on z-stack image data was used to conduct the Iba1+ channel. The thresholds for CD11c+ within Iba1+ pixels were determined by visual inspection and used for all images. For each z-stack image, the percentages of CD11c+ pixels within Iba1+ pixels were calculated. A similar procedure was used for measurement of percentage of LC3+, CD74+ microglia over total microglia population.

General Design of Behavioral tests. All behavioral testing was conducted during the light cycle, by a female experimenter blinded to experimental group. Equipment was cleaned with 2% chlorhexidine diacetate between animals. The order of tests conducted for each cohort were as follows: 1 h locomotor activity, elevated plus maze, visible platform trials of the water maze, submerged platform trials, and water maze probe trial.

One-hour Locomotor Activity and Open Field test. To evaluate general activity levels mice in were evaluated over a 1-h period in transparent (47.6×25.4×20.6 cm high) polystyrene enclosures. Each cage was surrounded by a frame containing a 4×8 matrix of photocell pairs, the output of which was fed to an on-line computer (Hamilton-Kinder). The system software (Hamilton-Kinder) was used to define a 33×11 cm central zone and a peripheral or surrounding zone that was 5.5 cm wide with the sides of the cage being the outermost boundary. This peripheral area extended along the entire perimeter of the cage. Variables that were analyzed included the total number of ambulations and rearing on hindlimbs, as well as the number of entries, the time spent, and the distance traveled in the center area as well as the distance traveled in the periphery surrounding the center.

Elevated Plus Maze (EPM). Briefly, EPM was conducted with a 5 min trial conducted in a dimly lit room using a standard mouse elevated plus maze from Kinder Scientific (Poway). The maze consisted of a black acrylic surface measuring 5x5 cm and elevated 63 cm above the floor equipped with photo beam pairs. Four arms (35 cm long, 5 cm wide; two open and two with 15 cm high walls) extended from a central area. The MotorMonitor software (Kinder Scientific) quantified beam breaks as duration, distance traveled, entries, and time at rest in each zone (closed arms, open arms and center area).

Morris water maze (MWM). Briefly, MWM was conducted with cued (visible platform), place (submerged platform), and probe trials in a galvanized steel pool, measuring 120 cm in diameter, and filled with opaque water (diluted nontoxic white tempera paint). The PVC escape platform measured 11.5 cm in diameter. A digital video camera connected to a PC computer and the computer software program ANY-maze (Stoelting Co) tracked the swimming pathway of the mouse to the escape platform and quantified path length, latency to find escape platform, and swimming speeds.

On two consecutive days, animals received four cued trials, separated by 1 h, during which a red tennis ball atop a rod was attached to the escape platform and served as a visual cue. To prevent spatial learning, the escape platform was moved to a different quadrant location for each trial. The mouse was released from the quadrant opposite to the platform location and allowed 60 s to locate the platform. Once the mouse found the platform, it was allowed to remain there for 10 s before being returned to its home cage. Three days following visible platform testing, the cue was removed from the platform, and it was submerged 1 cm under the water for the hidden platform tests. Animals received two blocks of two consecutive trials on five consecutive days, with an inter-trial interval between 30-90 s and approximately 2 h separating trial blocks. The escape platform remained in the same quadrant location for all trials and distal cues were placed on the walls of the room to support spatial learning. The mouse was released from a different location for each trial on each day. The mouse was allowed 60 s to find the escape platform and allowed to sit on it for 10 s before being returned to its home cage. Visible and submerged platform trials were combined into blocks of two or four trials for analyses, respectively. Following completion of submerged platform trials on the 5th day of training, the escape platform was removed from the pool and one 60 s probe trial was conducted to assess memory retention for the location of the platform.

Single-cell RNAseq analysis. 6-month-old of Syk^{fl/fl}, Syk^{DMG}, Syk^{fl/fl}-5xFAD and Syk^{DMG}-5xFAD, as well as Trem2^{+/+}-Trem2^{-/-}-5xFAD female mice were sacrificed for the sample preparation. After perfusion with cold PBS, cortices were dissociated, and CD45+ cells were sorted using FACS. All CD45+ cell libraries were prepared using the 10x Genomics Chromium Single Cell 3′ v3 Gene Expression Kit and sequenced on Illumina NovaSeq. Cell Ranger Software Suite (v6.0.0) from 10×Genomics was used for sample demultiplexing, barcode processing, and single-cell counting. Cellranger count was used to align samples to the reference genome GRCm39, quantify reads, and filter reads and barcodes. Only protein-coding genes, with the exception of Clec7a, were retained in the analysis. The Seurat (v4.0) package in R (v4.1.0) was used for downstream analysis. For quality control, cells with mitochondrial content 7.5% were removed. Cells with low UMI and gene number per cell were filtered out. Cutoffs for UMI and gene number were empirically determined on the basis of histograms showing cell density as a function of UMI per gene counts. Cutoffs of UMI greater than 2000 and less than 20000, and genes greater than 1000 were applied. Genes expressed in fewer than 10 cells were removed from the dataset. Microglia clusters were digitally isolated and further filtered for clusters containing doublets, stress genes, and apoptotic cells. After filtering, the dataset from Syk^{fl/fl}, Syk^{DMG}, Syk^{fl/fl}-5xFAD and Syk^{DMG}-5xFAD mice (n=3 for each genotype) showed in FIG. 17(A-L) and FIG. 22(A-D) (refer to as SYK ONLY) contained a total of 57,830 microglia cells with a median 5871 UMI and median 2537 genes and the integration dataset from Syk^{ne bdol/fl}-, Syk^{DMG}-, Trem2^{+/+}- and Trem2^{-/-}-5xFAD mice (n=3 for each genotype) showed in the FIG. 18(A-L) (refer to as SYK-TREM2) contained a total of 38,404 microglia cells remained with a median 5483 UMI and median 2359 genes per cell.

Clustering and differential expression analysis. For both SYK ONLY and SYK-TREM2 datasets, data were normalized using the SCTransform method regressed on mitochondrial gene percentage and integrated using FindlntegrationAnchors function and Canonical Correlation Analysis (CCA). Principle component analysis was performed, and the top 30 principal components were selected for dimensionality reduction using the Uniform Approximation and Projection (UMAP) algorithm. Clustering was performed using the FindNeighbors and FindClusters functions using a resolution of 0.6 or 0.4. Marker genes were identified by comparing each cluster against all other clusters using the FindAllMarkers function with default settings (log-fold change threshold of 0.25 and >10% cells expressing the gene). Cell clusters from each tissue were annotated based on marker gene expression. For data visualization and FIG. preparation, the scRNA-Seq data was also analyzed with BBrowser version 2.7.5 (SingleCell).

Pseudotime trajectory analysis. Single-cell pseudotime trajectories were inferred using the R package slingshot (v2.1.0). Normalized values resulting from batch integration were used as input for the calculation of cell lineages and pseudotime. Genes which were significantly differentially expressed along pseudotime were identified using the R package tradeSeq (v1.6). A negative binomial generalized additive model was fit to the identified lineages using the fitGAM function using the top 3000 variable features. Genes which changed significantly along the DAM trajectory were identified using association Test. Microglia from Syk^{DMG}-5xFAD were then compared against Trem2^{-/-}-5xFAD microglia along the DAM trajectory using the conditionTest function. DE genes (FDR <0.05) were further filtered by selecting those which overlapped with those that changed significantly along the DAM trajectory.

Anti-CLEC7A antibody production. Anti-mouse CLEC7A monoclonal antibody (mAb) 2A11 was used herein. A DNA fragment encoding heavy chain variable (VH) and CH1 regions was cloned into the pFUSEmIgG2A-Fc1 vector with L234A, L235A, P329G (LALA-PG) mutation, which blocks Fc binding. A DNA fragment encoding the light chain was cloned into Fc-null pFUSE-mIgG2A-Fc1 vector. The heavy chain and light chain plasmids were co-transfected into Expi293F cells (Thermo Scientific) for expression at mass ratio 1:2. When cell viability was below 50% (5-7 days), the supernatant was collected and filtered with 0.22-μm filters. The antibody was purified with protein A agarose (GoldBio). Final preps were concentrated to about 10 mg/mL into PBS and stored in 80° C. for future use.

qRT-PCR analyses. Total RNA was extracted from cortices of the left-brain hemispheres using miRNeasy Mini Kit (QIAGEN). After cDNA synthesis using a High-Capacity cDNA Reverse Transcription Kit (Applied Biosystems), quantitative PCR was performed using SYBR Green real-time PCR master mix (Thermo-Fisher) and LightCycler 96 detection system (Roche). The reference gene was actin and relative gene expression levels were determined by the ΔΔCt method. Gene expression was considered undetectable if Ct values were >35 cycles.

Quantification and Statistical Analysis. All graphs represent the mean of all samples in each group±SEM as indicated in the FIG. legends. Statistical analysis was performed using GraphPad Prism software. P<0.05 was considered significant difference between different conditions, determined by a two-tailed unpaired t test (Mann Whitney), one-way ANOVA with Tukey's multiple comparisons test or two-way ANOVA with Sidak's multiple comparisons test as indicated in the FIG. legends. Quantification of fluorescence microscopy, confocal microscopy and electron microscopy images were performed using Imaris, ImageJ and MATLAB.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that vary depending upon the desired properties sought to be obtained by a particular embodiment. In some embodiments, the numerical parameters are be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) are construed to cover both the singular and the plural, unless specifically noted otherwise. In some embodiments, the term “or” as used herein, including the claims, is used to mean “and/or” unless explicitly indicated to refer to alternatives only or to refer to the alternatives that are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and may also cover other unlisted steps. Similarly, any composition or device that “comprises,” “has” or “includes” one or more features is not limited to possessing only those one or more features and may cover other unlisted features.

All methods described herein are performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member is referred to and claimed individually or in any combination with other members of the group or other elements found herein. One or more members of a group are included in, or deleted from, a group for reasons of convenience or patentability. When any such inclusion or deletion occurs, the specification is herein deemed to contain the group as modified thus fulfilling the written description of all Markush groups used in the appended claims.

To facilitate the understanding of the embodiments described herein, a number of terms are defined below. The terms defined herein have meanings as commonly understood by a person of ordinary skill in the areas relevant to the present disclosure. Terms such as “a,” “an,” and “the” are not intended to refer to only a singular entity, but rather include the general class of which a specific example may be used for illustration. The terminology herein is used to describe specific embodiments of the disclosure, but their usage does not delimit the disclosure, except as outlined in the claims.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for treating a microglial dysfunction-associated neurodegenerative disease in a subject in need thereof, the method comprising: administering to the subject a therapeutically effective amount of a composition comprising a microglia receptor agonist that directly activates SYK.

2. The method of claim 1, wherein the microglial dysfunction-associated neurodegenerative disease is Alzheimer's disease.

3. The method of claim 1, wherein the subject has TREM2 deficient cells in the brain prior to administration of the composition.

4. The method of claim 1, wherein the microglial dysfunction-associated neurodegenerative disease is characterized by a mutation in Trem2 resulting in decreased microglial activity.

5. The method of claim 1, wherein administering the therapeutically effective amount of the composition results in one or more of improved microglial metabolic activity, decreased microglial autophagy, reduced neurite dystrophy, decreased cell death, improved microglia viability or improved microglia numbers.

6. The method of claim 5, wherein administering the therapeutically effective amount of the composition results in improved microglia clustering around amyloid beta plaques or reduced plaque-associated neurite dystrophy.

7. The method of claim 1, wherein the microglia receptor agonist that directly activates SYK comprises a CLEC7A agonist or pharmaceutically acceptable salt thereof.

8. The method of claim 1, wherein the subject is human.

9. A method of reversing neuronal damage in a subject having a microglial dysfunction-associated neurodegenerative disease, wherein the microglial dysfunction-associated neurodegenerative disease is characterized by a mutation in Trem2 affecting microglial functions, the method comprising: administering to the subject a therapeutically effective amount of a composition comprising a microglia receptor agonist that directly activates SYK.

10. The method of claim 9, wherein the subject has TREM2 deficient cells in the brain prior to administration of the composition.

11. The method of claim 9, wherein administering the therapeutically effective amount of the composition results in one or more of improved microglial metabolic activity, decreased microglial autophagy, reduced neurite dystrophy, decreased cell death, improved microglia viability or improved microglia numbers.

12. The method of claim 9, wherein the microglial dysfunction-associated neurodegenerative disease is Alzheimer's disease.

13. The method of claim 11, wherein administering the therapeutically effective amount of the composition results in improved microglia clustering around amyloid beta plaques or reduced plaque-associated neurite dystrophy.

14. The method of claim 9, wherein the microglia receptor agonist that directly activates SYK comprises a CLEC7A agonist or pharmaceutically acceptable salt thereof.

15. The method of claim 9, wherein the subject is human.

16. A method of treating at least one symptom of cognitive dysfunction in a subject having a microglial dysfunction-associated neurodegenerative disease, wherein the microglial dysfunction-associated neurodegenerative disease is characterized by a mutation in Trem2 affecting microglial functions, the method comprising: administering to the subject a therapeutically effective amount of a composition comprising a microglia receptor agonist that directly activates SYK or activates at least one ITAM pathway.

17. The method of claim 16, wherein the at least one symptom is selected from a short term memory function and a spatial learning dysfunction.

18. The method of claim 16, wherein the subject has TREM2 deficient cells in the brain prior to administration of the composition.

19. The method of claim 16, wherein the microglia receptor agonist that directly activates SYK comprises a CLEC7A agonist or pharmaceutically acceptable salt thereof.

20. The method of claim 16, wherein the subject is human.


SEQUENCE LISTING (8 sequences)

SEQ ID NO: 1 - DNA, 23 bp, Artificial Sequence
  SYNTHESIZED
cctacacgacgctcttccgatct

SEQ ID NO: 2 - DNA, 22 bp, Artificial Sequence
  SYNTHESIZED
agatcggaagagcacacgtctg

SEQ ID NO: 3 - DNA, 19 bp, Artificial Sequence
  SYNTHESIZED
cttccccactggcctcaag

SEQ ID NO: 4 - DNA, 20 bp, Artificial Sequence
  SYNTHESIZED
ccaaaacccagtgatccagc

SEQ ID NO: 5 - DNA, 19 bp, Artificial Sequence
  SYNTHESIZED
tgctagccgcaggcattac

SEQ ID NO: 6 - DNA, 21 bp, Artificial Sequence
  SYNTHESIZED
gggtgcccaaagaatcagaac

SEQ ID NO: 7 - DNA, 18 bp, Artificial Sequence
  SYNTHESIZED
ggagggggttgaggtgtt

SEQ ID NO: 8 - DNA, 23 bp, Artificial Sequence
  SYNTHESIZED
tgtgcacttttattggtctcaag